SEQUENCE LISTING (6 sequences)

SEQ ID NO: 1 - protein, 10 aa, Artificial sequence
  recombinant/synthetic peptide
ACDCRGDCFC

SEQ ID NO: 2 - protein, 14 aa, Artificial sequence
  recombinant/synthetic peptide
KLAKLAKKLAKLAK

SEQ ID NO: 3 - DNA, 22 bp, Rattus norvegicus
gaccctcggacaactgtgtctt

SEQ ID NO: 4 - DNA, 22 bp, Rattus norvegicus
gcgtttgtagaagcaacagtga

SEQ ID NO: 5 - DNA, 20 bp, Rattus norvegicus
tggcacagtcaaggctgaga

SEQ ID NO: 6 - DNA, 20 bp, Rattus norvegicus
cttctgagtggcagtgatgg

(19) United States
(12) Patent Application Publication
(10) Pub. No.: US 2018/0000791 A1
(43) Pub. Date: Jan. 4, 2018

(54) METHODS AND COMPOSITIONS FOR TREATING CONDITIONS ASSOCIATED WITH ANGIOGENESIS USING A VASCULAR ADHESION PROTEIN-1 (VAP-1) INHIBITOR

(71) Applicant: MASSACHUSETTS EYE & EAR INFIRMARY, Boston, MA (US)

(72) Inventor: Ali Hafezi-Moghadam, Jamaica Plain, MA (US)

(73) Assignee: MASSACHUSETTS EYE & EAR INFIRMARY, Boston, MA (US)

(21) Appl. No.: 15/429,679

(22) Filed: Feb. 10, 2017

Related U.S. Application Data
(63) Continuation of application No. 14/936,627, filed on Nov. 9, 2015, now abandoned.
Application No. 14/936,627 is a continuation of application No. 14/070,069, filed on Nov. 1, 2013, now abandoned.
Application No. 14/070,069 is a continuation of application No. 13/307,920, filed on Nov. 30, 2011, now abandoned.
Application No. 13/307,920 is a continuation of application No. 12/265,521, filed on Nov. 5, 2008, now abandoned.
(60) Provisional application No. 60/985,848, filed on Nov. 6, 2007.

(51) Int. Cl. A61K 31/426; C07K 16/22; A61K 45/06; A61K 31/06; A61K 39/395; A61K 31/7088; C12N 15/115; A61N 5/06

(52) U.S. Cl. CPC A61K 31/426; C12N 15/115; C07K 16/22; A61N 5/062; A61K 31/06; A61K 39/3955; A61K 31/7088; A61K 45/06

(57) ABSTRACT

The invention relates generally to methods and compositions for treating conditions associated with angiogenesis, and, more specifically, the invention relates to methods and compositions for treating conditions associated with angiogenesis using vascular adhesion protein-1 (VAP-1) inhibitors. The invention also relates to methods and compositions for treating conditions associated with lymphangiogenesis using VAP-1 inhibitors.

### CROSS-REFERENCE TO RELATED APPLICATIONS

This application is a continuation of U.S. application Ser. No. 14/936,627, filed Nov. 9, 2015, which is a continuation of U.S. application Ser. No. 14/070,069, filed Nov. 1, 2013, which is a continuation of U.S. application Ser. No. 13/307,920, filed Nov. 30, 2011, which is a continuation of U.S. application Ser. No. 12/265,521, filed Nov. 5, 2008, which claims priority to and the benefit of U.S. Provisional Patent Application No. 60/985,848, filed Nov. 6, 2007, the disclosure of each of which is incorporated herein by reference in its entirety.

### FIELD OF THE INVENTION

The invention relates generally to methods and compositions for treating conditions associated with angiogenesis, and, more specifically, the invention relates to methods and compositions for treating conditions associated with angiogenesis using vascular adhesion protein-1 (VAP-1) inhibitors. The invention also relates to methods and compositions for treating conditions associated with lymphangiogenesis using VAP-1 inhibitors.

### BACKGROUND

Blood vessels supply oxygen and nutrients to and remove waste products from living tissue. Angiogenesis refers to the biological process in which blood vessels are formed. Angiogenesis is an essential part of biological processes, for example, reproduction, embryonic development, and wound repair. However, angiogenesis normally occurs in humans and animals in a very limited set of circumstances.

Angiogenesis and the rate of angiogenesis involve changes in the local equilibrium between positive and negative regulators of the growth of microvessels. Abnormal angiogenesis occurs when the body loses at least some control of this equilibrium, resulting, for example, in either excessive or insufficient blood vessel growth. For example, the absence of angiogenesis normally required for natural healing conditions can lead to conditions such as ulcers, strokes, and heart attacks. In contrast, excessive blood vessel proliferation has been associated with cancer, tumor growth, tumor spread (metastasis), psoriasis, rheumatoid arthritis, and conditions associated with ocular neovascularization, such as corneal neovascularization and choroidal neovascularization.

Thus, there are some instances where a greater degree of angiogenesis is desirable—increasing blood circulation, wound healing, and ulcer healing. For example, researchers have investigated the use of recombinant angiogenic growth factors, such as fibroblast growth factor (FGF) family, endothelial cell growth factor (ECGF), and more recently, vascular endothelial growth factor (VEGF) to induce collateral artery development in animal models of myocardial and hindlimb ischemia.

However, there also are many instances in which inhibition of angiogenesis and/or regression of blood vessels is desirable. For example, many diseases are driven by persistent unregulated angiogenesis, also sometimes referred to as “neovascularization.” Many solid tumors are vascularized as a result of angiogenesis such that the neovascularization provides the tumors with a sufficient supply of oxygen and nutrients that permit them to grow rapidly and metastasize. Thus, tumor growth and metastasis are angiogenesis-dependent. A tumor must continuously stimulate the growth of capillary blood vessels for the tumor itself to grow. In arthritis, capillary blood vessels invade the joint and destroy cartilage. In diabetes, capillaries invade the vitreous of the eye, bleed, and cause blindness.

In ocular disorders, neovascularization is the most common cause of blindness. One form of ocular neovascularization is corneal neovascularization. Corneal neovascularization is associated with excessive blood vessel ingrowth into the cornea from the limbal vascular plexus. Since the cornea normally is devoid of blood and lymphatic vessels, oxygen supply to the cornea normally is supplied from the air. When the normal supply of oxygen from the air to the cornea is altered, for example by use of contact lenses, the equilibrium the local equilibrium between positive and negative regulators that controls growth of microvessels can shift to favor neovascularization of the cornea. Severe cases of corneal neovascularization can result in blindness.

Another form of ocular neovascularization is choroidal neovascularization (CNV). Choroidal neovascularization can lead to hemorrhage and fibrosis, with resulting visual loss in a number of conditions of the eye, including, for example, age-related macular degeneration, ocular histoplasmosis syndrome, pathologic myopia, angioid streaks, idiopathic disorders, choroiditis, choroidal rupture, overlying choroid nevi, and certain inflammatory diseases. One of the disorders, namely, age-related macular degeneration (AMD), is the leading cause of severe vision loss in people aged 65 and above (Bressler et al. (1988) Surv. Ophthalmol. 32, 375-413, Guyer et al. (1986) Arch. Ophthalmol. 104, 702-705, Hyman et al. (1983) Am. J. Epidemiol. 188, 816-824, Klein & Klein (1982) Arch. Ophthalmol. 100, 571-573, Leibowitz et al. (1980) Surv. Ophthalmol. 24, 335-610). Although clinicopathologic descriptions have been made, little is understood about the etiology and pathogenesis of AMD.

Dry AMD is the more common form of the disease, characterized by drusen, pigmentary and atrophic changes in the macula, with slowly progressive loss of central vision. Wet or neovascular AMD is characterized by subretinal hemorrhage, fibrosis and fluid secondary to the formation of choroidal neovasculature, and more rapid and pronounced loss of vision. While less common than dry AMD, neovascular AMD accounts for 80% of the severe vision loss due to AMD. Approximately 200,000 cases of neovascular AMD are diagnosed yearly in the United States alone.

Currently, treatment of the dry form of age-related macular degeneration includes administration of antioxidant vitamins and/or zinc. Treatment of the wet form of age-related macular degeneration, however, has proved to be more difficult. Currently, two separate methods have been approved in the United States of America for treating the wet form of age-related macular degeneration. These include laser photocoagulation and photodynamic therapy (PDT) using a benzoporphyrin derivative photosensitizer. During laser photocoagulation, thermal laser light is used to heat and photocoagulate the neovasculature of the choroid. A problem associated with this approach is that the laser light must pass through the photoreceptor cells of the retina in order to photocoagulate the blood vessels in the underlying choroid. As a result, this treatment destroys the photoreceptor cells of the retina creating blind spots with associated vision loss. During photodynamic therapy, a benzoporphyrin derivative photosensitizer is administered to the individual to be treated. Once the photosensitizer accumulates in the choroidal neovasculature, non-thermal light from a laser is applied to the region to be treated, which activates the photosensitizer in that region. The activated photosensitizer generates free radicals that damage the vasculature in the vicinity of the photosensitizer (see, U.S. Pat. Nos. 5,798,349 and 6,225,303). This approach is more selective than laser photocoagulation and is less likely to result in blind spots. Under certain circumstances, this treatment has been found to restore vision in patients afflicted with the disorder (see, U.S. Pat. Nos. 5,756,541 and 5,910,510).

During clinical studies, however, it has been found that recurrence of neovascularization and/or vessel leakage can occur post-PDT-treatment. Increasing photosensitizer or light doses do not appear to prevent this recurrence, and can even lead to undesired non-selective damage to retinal vessels (Miller et al. (1999) Archives of Ophthalmology 117: 1161-1173). Another avenue of investigation is to repeat the PDT procedure over prolonged periods of time. The necessity for repeated PDT treatments can nevertheless be expected to lead to cumulative damage to the retinal pigment epithelium (RPE) and choriocapillaris, which may lead to progressive treatment-related vision loss. PDT also can cause transient visual disturbances, injection-site adverse effects, transient photosensitivity reactions, infusion-related back pain, and vision loss.

To address some of the issues associated with PDT, the PDT treatment can be combined with administration of anti-angiogenesis factors, for example, MACUGEN® or LUCENTIS®. However, new treatments to address CNV, both alone and in combination with PDT, are needed.

The current treatments of diseases associated with unwanted angiogenesis, namely cancer, corneal neovascularization, and CNV, are inadequate. Thus, identification of agents that inhibit angiogenesis such as by inhibiting blood vessel formation and/or inducing regression of blood vessels is needed. In addition, some of these diseases, such as cancer and new vessel growth in the cornea, are also associated with lymphangiogenesis, the growth of lymph vessels. Accordingly, identification of agents that inhibit lymphangiogenesis such as by inhibiting lymph vessel formation and/or inducing regression of lymph vessels is needed.

Vascular adhesion protein-1 (VAP-1), a 170-kDa homodimeric sialylated glycoprotein, is an endothelial adhesion molecule involved in the leukocyte recruitment cascade. VAP-1 was originally discovered in inflamed synovial vessels, but it is also expressed on the endothelium of other tissues such as skin, brain, lung, liver and heart under normal and inflamed conditions.

VAP-1 acts as both an adhesion molecule and an enzyme. In its function as an adhesion molecule, it mediates leukocyte adhesion and transmigration. In its function as an enzyme, it generates reactive oxygen species and other agents, which are highly injurious to the vascular endothelium and potentially also other cells, such as neurons.

Previous studies have revealed that VAP-1 is identical with the cell-surface enzyme, semicarbazide-sensitive amine oxidase (SSAO), which catalyzes the deamination of primary amines, such as methylamine and aminoacetone. This reaction generates toxic formaldehyde and methylglyoxal, hydrogen peroxide and ammonia, which are known as reactive chemicals and major reactive oxygen species. Previously, SSAO activity has been detected in retinal tissues in connection with vascular permeability. Accordingly, VAP-1 inhibitors have been investigated in connection with vascular hyperpermeable diseases and inflammatory conditions. See, for example, PCT Publication Nos. WO 2004/087138 (nationalized in the United States as U.S. Published Application No. 2006/0229346), WO 2004/067521, WO 2005/089755, and U.S. Pat. Nos. 7,125,901, 6,624,202, 6,066,321, and 5,580,780.

### SUMMARY OF THE INVENTION

The present invention relates, in part, to the discoveries that VAP-1 plays a role in angiogenesis and that VAP-1 blockade inhibits angiogenesis in animal models. The present invention is directed to methods and compositions for treating conditions associated with unwanted angiogenesis, also referred to as neovascularization, using a VAP-1 inhibitor. In one aspect, the invention provides a method of treating an angiogenic condition. The method includes administering a VAP-1 inhibitor to a subject in an amount sufficient to inhibit angiogenesis. The angiogenic condition may be, for example, cancer, diabetes, diabetic retinopathy, age-related macular degeneration, rheumatoid arthritis, psoriasis, complications of AIDS (Kaposi's sarcoma), Alzheimer's disease, chronic inflammatory diseases (i.e. Crohn's disease and ulcerative colitis), acute inflammation, rheumatic diseases, autoimmune diseases, systemic inflammatory diseases including systemic lupus erythematosus (SLE), systemic sclerosis (SSc), Sjögren's syndrome (SS), mixed connective tissue disease (MCTD), polymyositis/dermatomyositis (PM/DM) and systemic vasculitis, endometriosis, skin diseases (i.e. psoriasis), thrombotic diseases (including diseases related to platelet function), and/or diseases related to coagulation and complement cascade. Particularly, the condition may include cancer, an ocular angiogenic condition such as unwanted choroidal neovasculature or corneal angiogenesis, scar formation, tissue repair, wound healing, atherosclerosis, and/or arthritis.

Accordingly, in another aspect, the invention provides a method for treating cancer. The method includes administering a VAP-1 inhibitor to a subject in an amount sufficient to inhibit angiogenesis. In certain embodiments, the angiogenesis inhibition attenuates tumor growth and/or inhibits tumor metastasis.

In another aspect, the invention provides a method for treating an ocular angiogenic condition. The method includes administering a VAP-1 inhibitor to a subject in an amount sufficient to inhibit angiogenesis of the eye. For example, the invention provides a method for treating unwanted choroidal neovasculature, which includes administering a VAP-1 inhibitor to a subject in an amount sufficient to inhibit the unwanted choroidal neovasculature. The subject may have age-related macular degeneration. The invention also provides a method of treating corneal angiogenesis, which includes administering a VAP-1 inhibitor to a subject in an amount sufficient to inhibit the unwanted corneal angiogenesis.

It is contemplated that inhibition of angiogenesis (such as inhibition of unwanted tumor-related neovasculature, choroidal neovasculature, or corneal neovasculature) may include blood vessel regression and/or inhibition of blood vessel formation. Inhibition of blood vessel formation may include cessation of blood vessel formation or a decrease in the rate of blood vessel growth in a treated subject as compared to an untreated subject. Moreover, it is contemplated that the VAP-1 inhibitor may be administered locally or systemically.

The present invention also relates, in part, to the discovery that VAP-1 blockade inhibits lymphangiogenesis in animal models. Accordingly, the present invention also is directed to methods and compositions for treating conditions associated with unwanted lymphangiogenesis using a VAP-1 inhibitor. In one aspect, the invention provides a method of treating a lymphangiogenic condition. The method includes administering a VAP-1 inhibitor to a subject in an amount sufficient to inhibit lymphangiogenesis. The lymphangiogenic condition may be, for example, cancer, neoplasm, metastasis, organ transplantation, particularly the organization of immunologically active lymphocytic infiltrates following organ transplantation, edema, rheumatoid arthritis, scar formation, tissue repair, psoriasis, and wound healing. Particularly, the condition may include cancer or an ocular lymphangiogenic condition such as corneal lymphangiogenesis.

In another aspect, the invention provides a method for treating cancer. The method includes administering a VAP-1 inhibitor to a subject in an amount sufficient to inhibit lymphangiogenesis. In certain embodiments, the lymphangiogenesis inhibition attenuates tumor growth and/or inhibits tumor metastasis.

In another aspect, the invention provides a method for treating an ocular lymphangiogenic condition. The method includes administering a VAP-1 inhibitor to a subject in an amount sufficient to inhibit lymphangiogenesis of the eye. For example, the invention provides a method for treating corneal lymphangiogenesis, which includes administering a VAP-1 inhibitor to a subject in an amount sufficient to inhibit the unwanted corneal lymphangiogenesis.

It is contemplated that inhibition of lymphangiogenesis (such as inhibition of unwanted tumor-related lymph vessels or corneal lymphangiogenesis) may include lymph vessel regression and/or inhibition of lymph vessel formation. Inhibition of lymph vessel formation may include cessation of lymph vessel formation or a decrease in the rate of lymph vessel growth in a treated subject as compared to an untreated subject. Moreover, it is contemplated that the VAP-1 inhibitor may be administered locally or systemically.

A variety of VAP-1 inhibitors may be used in the invention. Useful VAP-1 inhibitors, include but are not limited to, for example, anti-VAP-1 neutralizing antibody (available, for example, from R&D Systems, Minneapolis, Minn., catalogue nos. AF3957, MAB39571, and MAB3957; Everest Biotech, Oxford, United Kingdom, catalogue no. EB07582; and antibodies identified in U.S. Pat. Nos. 4,704,692; 6,066,321 and 5,580,780 and Koskinen et al. (2004) BLOOD 103:3388; Arvilommi et al. (1996) EUR. J. IMMUNOL. 26:825, Salmi et al. (1993) J. EXP. MED., 178:2255, and Kirten et al. (2005) EUR. J. IMMUNOL. 35:3119); small molecules such as phenylhydrazine, 5-hydroxytryptamine, 3-bromopropylamine, N-(phenyl-allyl)-hydrazine HCl (LJP-1207), 2-hydrazinopyridine, MDL-72274 ((E)-2-phenyl-3-chloroallylamine hydrochloride), MDL-72214 (2-phenylallylamine), mexiletine, isoniazid, imipramine, maprotiline, zimeldine, nomifensine, azoprocarbazine, monomethylhydrazine, d1-alpha methyltryptamine, d1-alpha methylbenzylamine, MD780236 (Dostert et al. (1984), J. PHARMACY & PHARMACOL., 36:782), 2-(dimethyl(2-phenylethyl)silyl) methanamine, cuprozine, alkylamino derivatives of 4-amniomethylpyridine (Bertini et al. (2005) J. MED. CHEM. 48:664), kynuramine, those identified in PCT Publication Nos. WO 2004/087138 (nationalized in the United States as U.S. Published Application No. 2006/0229346), WO 2004/067521. WO2005/014530, and WO 2005/089755, in U.S. Published Application Nos. 2004/0236108, 2004/0259923, 2005/0096360, and 2006/0025438, and in U.S. Pat. Nos. 7,125,901 and 6,624,202, and small molecules that bind VAP-1 to prevent or reduce its binding to its cognate receptor or ligand; peptides (for example, the peptide inhibitors discussed in Yegutkin et al. (2004) EUR. J. IMMUNOL. 34:2276 and Wang et al. (2006) J. MED. CHEM. 49:2166); nucleic acids (for example, anti-VAP-1 aptamers and siRNAs identified in PCT Publication No. WO2006/134203); certain antibodies, antigen binding fragments thereof, and peptides that bind preferentially to VAP-1 or the VAP-1 cognate receptor or ligand; antisense nucleotides and double stranded RNA for RNAi that ultimately reduce or eliminate the production of either VAP-1 or its cognate receptor or ligand; soluble VAP-1; and/or soluble VAP-1 cognate receptor or ligand. These VAP-1 inhibitors can act as direct or indirect inhibitors of angiogenesis and/or lymphangiogenesis.

In any aspect of the invention, the method may include additional treatment and/or administration of additional agents, before, during and/or after administration of the VAP-1 inhibitor. For example, photodynamic therapy treatment, administration of a VEGF inhibitor, and/or administration of an apoptosis-modulating factor, may be performed before, during, and/or after administration of one or more VAP-1 inhibitors. The practice of this method may enhance, additively and/or synergistically, the therapeutic efficacy of the VAP-1 inhibitor and/or additional treatment and/or additional agent.

### BRIEF DESCRIPTION OF THE FIGURES

The foregoing and other objects, features, and advantages of the present invention, as well as the invention itself, may be more fully understood from the following description of preferred embodiments, when read together with the accompanying drawings.

FIG. 1A shows a gel depicting retinal and choroidal VAP-1 mRNA expression relative to GAPDH mRNA expression. RT-PCR amplification of VAP-1 mRNA in the retinal and choroidal tissues was obtained from normal rats. FIG. 1B is a chart showing averages from densitometric analysis of the mRNA bands for VAP-1, normalized to the values of GAPDH mRNA expression. Values are expressed as mean±SEM (n=4 in each group). †, p<0.01.

FIGS. 2A-2D are representative photomicrographs showing localization of VAP-1 in the choroid. FIG. 2A is a phase-contrast photomicrograph of the choroidal-scleral complex. FIG. 2B is a fluorescent micrograph of choroidal tissues immunostained for VAP-1 (ALEXA FLUORE® 546). FIG. 2C is a photomicrograph with counterstaining for nuclei with DAPI. FIG. 2D shows the merged images of FIGS. 2B and 2C. The arrows in FIGS. 2B and 2D indicate VAP-1 positive staining in the choroidal vessels. Bar=100 μm.

FIGS. 3A-3D are representative micrographs showing tissue localization of VAP-1 in a representative CNV lesion. FIG. 3A is a fluorescent micrograph of a laser-induced CNV lesion, immunostained with isolectin B4. FIG. 3B is a fluorescent micrograph of rat choroid, immunostained for VAP-1 (ALEXA FLUORE® 546). FIG. 3C is a photomicrograph with counterstaining for nuclei with DAPI. FIG. 3D shows the merged images of FIGS. 3B and 3C. The arrows in FIGS. 3B and 3D indicate the localization of VAP-1 in the CNV and choroidal vessels. Bar=50 μm.

FIGS. 4A and 4B depict the impact of VAP-1 Blockade on CNV Formation. FIG. 4A shows representative micrographs of CNV lesions in the choroidal flatmounts from an animal treated with vehicle or VAP-1 inhibitor. The dashed lines show the extent of the CNV lesions filled with FITC-dextran in flatmounted choroids. Bar=100 μm. FIG. 4B shows a quantitative analysis of CNV size. Bars show the average of CNV size in each group. Values are mean±SEM (n=7 to 9). †, p<0.01.

FIGS. 5A-5B show representative fluorescein angiographs of CNV lesions. FIG. 5A shows early-phase (1-2 minutes) and late-phase (6-8 minutes) fluorescein angiograms of the animals treated with vehicle or VAP-1 inhibitor. Fluorcscein angiography was performed at day 7 after laser photocoagulation and the VAP-1 inhibitor treatment. Arrows indicate the respective grades of the various lesions. FIG. 5B is a graph showing the percentage of lesions graded as 0, I, IIA and IIB in vehicle-treated (n=11) and inhibitor-treated animals (n=12).

FIGS. 6A and 6B depict the effect of VAP-1 blockade on macrophage infiltration in CNV lesions. FIG. 6A shows representative micrographs of CNV lesions, immunostained for ED-1, in animals treated with vehicle or VAP-1 inhibitor. In FIG. 6A, the staining shown as light areas indicates ED-1 positive cells (macrophages), while the staining shown as darker areas (but lighter than the background) shows nuclear staining with DAPI. FIG. 6B is a graph showing the ED1-positive cells (macrophages) detected in the RPE-choroid laser lesions at day 1 through 7 after laser injury, with a peak at day 3. The index was normalized to peak response (day 3) of vehicle-treated animals. Values are mean±SEM (n=4 at each time point). *, p<0.05.

FIGS. 7A-7C are graphs showing the impact of VAP-1 blockade on inflammation-associated molecules: TNF-α (FIG. 7A), MCP-1 (FIG. 7B) and ICAM-1 (FIG. 7C). Bars indicate the average protein levels of respective inflammation-associated molecule in the RPE-choroidal complex obtained from laser-induced CNV animals (CNV) treated with vehicle or VAP-1 inhibitor at 3 days after laser photocoagulation. Values are mean±SEM (n=8 to 12). *, p<0.05. CTR indicates control animals that were not subjected to laser-induced CNV.

FIG. 8 is a schematic view of the role of VAP-1 in laser-induced CNV formation.

FIG. 9 is a schematic view of the method used to induce corneal neovascularization in mice using hydron pellets (0.3 μl) containing 30 ng mouse IL-1β (401-ML; R&D Systems). The pellets were implanted into mouse corneas to induce corneal neovascularization.

FIG. 10A is a set of photographs depicting the impact of VAP-1 inhibition on IL-1β-induced corneal angiogenesis, at 2, 4, and 6 days after pellet implantation.

FIG. 10B is a graph showing the neovascular area in corneas at 6 days following IL-1β-induced corneal angiogenesis, for mice treated with IL-1β, IL-1β+vehicle, or IL-1β+VAP-1 inhibitor.

FIGS. 11A and 11B depict the impact of VAP-1 inhibition on CD11b(+) cells in IL-1β-induced corneal angiogenesis, at 3 days after pellet implantation. FIG. 11A is a set of photomicrographs showing CD11b(+) cells in corneas treated with IL-1β, IL-1β+vehicle, or IL-1β+VAP-1 inhibitor. FIG. 11B is a graph comparing the number of CD11b(+) cells appearing in IL-1β-implanted cornea with and without VAP-1 inhibition, at 3 days after pellet implantation.

FIG. 12 depicts the impact of VAP-1 inhibition on Gr-1(+) cells, which are indicative of neutrophils and macrophages, and F4/80(+) cells, which are indicative of monocytes and macrophages, in IL-1β-induced corneal angiogenesis. The left side of FIG. 12 is a set of photomicrographs showing F4/80(+) cells and Gr-1(+) cells in corneas treated with IL-1β, IL-1β+vehicle, or IL-1β+VAP-1 inhibitor. The right side of FIG. 12 shows graphs comparing the number of Gr-1(+) cells and F4/80(+) cells, respectively, appearing in IL-1β-implanted cornea with and without VAP-1 inhibition, following implantation. The top graph indicates that VAP-1 reduces Gr-1(+) cells (neutrophils and macrophages). The bottom graph indicates that VAP-1 reduces F4/80(+) cells (monocytes and macrophages).

FIG. 13 shows a set of photographs of corneal tissue samples following induction of corneal lymphangiogenesis with IL-1β and treatment with vehicle (IL-1β+Vehicle) or VAP-1 (IL-1β+VAP-1inh.). Anti-LYVE-1 stain identifies lymphatic vessels. As shown in FIG. 13, VAP-1 inhibitor reduces growth of lymphatic vessels.

FIG. 14A shows a set of photographs of untreated corneal tissue (no IL-1β treatment). Samples in the top two photographs were stained with anti-CD31 to identify endothelial cells in blood vessels. Samples in the middle two photographs were stained with anti-VAP-1 to identify the presence of VAP-1. The bottom two photographs show merger of the two photographs above and indicate that VAP-1 is expressed on quiescent blood vessels. FIG. 14B also shows a set of photographs of untreated corneal tissue (no IL-1β treatment). However, samples in the top two photographs were stained with anti-LYVE-1 to identify lymphatic vessels. Samples in the middle two photographs were stained with anti-VAP-1 to identify the presence of VAP-1. The bottom two photographs shows merger of the two photographs above it and indicate that VAP-1 is not expressed on quiescent lymphatic vessels.

FIG. 15 shows a set of photographs of corneal tissue from corneas treated with IL-1β to induce angiogenesis. Samples in the top three photographs were stained with anti-CD31 to identify endothelial cells in blood vessels. Samples in the middle three photographs were stained with anti-VAP-1 to identify the presence of VAP-1. The bottom three photographs shows merger of the two photographs above it and indicates that VAP-1 is expressed on angiogenic blood vessels.

FIGS. 16A and 16B show VAP-1 immunostaining in the posterior segment of the eye. FIG. 16A shows paraffin sections of normal human eyes stained with non-immune isotype-matched control mAb. FIG. 16B shows paraffin sections of normal human eyes stained with anti VAP-1 mAb. Arrows depict VAP-1 expression on the vessels. Magnification is 50×. ON stands for optic nerve head. FIG. 16C shows paraffin sections of normal human eyes stained with anti VAP-1 mAb. Arrows depict VAP-1 expression on the smooth muscle cells of the ciliary body. Magnification is 200×.

FIG. 17 shows demographic data and case information for the subjects donating tissue for the experiments of Example 5. Abbreviations are: N/A, not available; CVD, cardiovascular disease; ICH, intracerebral hemorrhage; SLE, systemic lupus erythematosus; and HTN, hypertension.

FIG. 18 shows a summary of VAP-1 expression in different ocular tissues divided into arteries and veins. “Ø” means the tissue was not available, and “−,” “+,” and “++” refer to the intensity of VAP-1 staining ranging from no staining to some staining to most staining, respectively.

FIGS. 19A-F show AEC (3-Amino-9-ethylcarbazole) staining of VAP-1 in various ocular tissues. VAP-1 staining was evaluated in different tissues and selectively found in choroidal (FIGS. 19C and 19D) and scleral vessels (FIGS. 19E and 19F), but not in iris vessels (FIGS. 19A and 19B). Magnification: FIG. 19A, FIG. 19C and FIG. 19E, 100×; FIG. 19B, FIG. 19D, and FIG. 19F, 320×.

FIGS. 20A-C show AEC staining of VAP-1 in various ocular tissues. VAP-1 is strongly expressed in vessels of neuronal tissues: the retina (FIGS. 20A and 20B) and the optic nerve (FIG. 20C). Magnification: FIG. 20A and FIG. 20C, 100×; FIG. 20B, 200×.

FIGS. 21A and 21B show quantification of VAP-1 expression in arteries and veins, respectively, in various ocular tissues. Highest levels of VAP-1 expression were found in the arteries of the retina and optic nerve (FIG. 21A). VAP-1 was not detectable in arteries (FIG. 21A) and veins (FIG. 21B) of the iris.

FIGS. 22A and 22B show a comparison of VAP-1 expression in the arteries and veins of choroidal vessels. FIG. 22A shows quantification of VAP-1 expression in the choroidal vessels. VAP-1 expression was significantly higher in arteries than veins. FIG. 22B shows a representative micrograph of VAP-1 staining in the choroidal vessels, indicating the differences in VAP-1 expression (arrows) between arteries and veins. Magnification: 360×.

FIGS. 23A-E show cellular localization of VAP-1 in ocular vessels. Paraffin sections were stained with antibodies against endothelial CD31 (FIGS. 23A and 23B), smooth muscle actin (FIGS. 23C and 23D), and VAP-1 (FIG. 23E). VAP-1 colocalized in both endothelial and smooth muscle cells (FIG. 23E). Magnification: FIG. 23A and FIG. 23C, 160×;

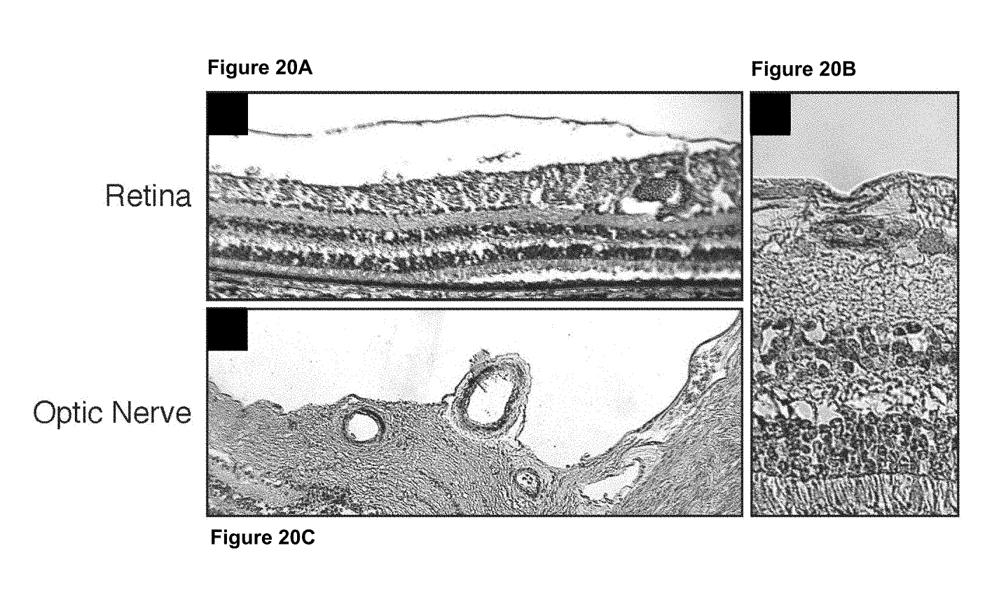

FIG. 20B, FIG. 20D and FIG. 20E, 640×.

### DETAILED DESCRIPTION OF THE INVENTION

The present invention relates, in part, to the discoveries that VAP-1 plays a role in angiogenesis and that VAP-1 blockade inhibits angiogenesis in animal models, for example, animal models of CNV and corneal angiogenesis. Accordingly, the invention describes methods and compositions for treating angiogenic conditions by administering a VAP-1 inhibitor to a subject in an amount sufficient to inhibit angiogenesis. Inhibition of angiogenesis using a VAP-1 inhibitor can include blood vessel regression and/or inhibition of blood vessel formation. Inhibition of new blood vessel formation includes cessation of new blood formation and/or a decrease in the rate of new blood vessel formation, for example, as compared to an untreated control.

VAP-1 inhibition of the present invention may be useful in inhibiting various types of angiogenesis, for example, sprouting angiogenesis, intussusceptive angiogenesis, and/or inflammatory angiogenesis. Sprouting angiogenesis enables new vessel growth across gaps in the vasculature. It is initiated by degradation of the basement membrane supporting endothelial cells by proteases secreted from the endothelial cells. The proteases may be secreted from endothelial cells activated by mitogens, such as vascular endothelial growth factor (VEGF) and basic fibroblast growth factor (bFGF). The endothelial cells loosened from the degraded basement membrane are free to migrate and proliferate, leading to the formation of endothelial cell sprouts in the stroma. Then, vascular loops are formed and capillary tubes develop to complete the lumen of the vessel and new basement membrane is deposited. Sprouting differs from intussusceptive angiogenesis because it forms a new vessel as opposed to splitting existing vessels.

Intussusceptive or splitting angiogenesis occurs when the capillary wall grows into the lumenal space to split a single vessel in two. After the two opposing capillary walls contact one another, the endothelial cell junctions are reorganized and the vessel bilayer is perforated to allow growth factors and cells to penetrate the lumen. Then, the core is formed between the two new vessels at the zone of contact. Specifically, pericytes and myofibroblasts facilitate deposition of collagen fibers into the core to provide an extracellular matrix for growth of the vessel lumen. By reorganizing existing cells in a blood vessel, intussusception allows for an increase in the number of capillaries without a corresponding increase in the number of endothelial cells. This is especially important in embryonic development as there are not enough resources to create a rich microvasculature with new cells every time a new vessel develops.

Inflammatory angiogenesis occurs as a result of specific compounds inducing the creation of new blood vessels, for example new capillaries, in the body. The absence of blood vessels in a repairing or otherwise metabolically active tissue may retard repair or some other function, and inflammatory angiogenesis acts to deliver new blood vessels to such tissue. Accordingly, tumor growth and metastasis may depend on inflammatory angiogenesis.

Inflammatory angiogenesis produces blood vessels where there previously were none, which can affect the properties of the newly vascularized tissue and inhibit the proper function of the tissue. For example, the use of contact lenses may cause tissue irritation and inflammation that may lead to neovascularization. Corneal neovascularization associated with contact lens use may inhibit the proper functioning of the corneal tissue. Moreover, choroidal neovascularization of the macula that is associated with AMD may inhibit the proper functioning of the macula. Since VAP-1 is involved in the leukocyte recruitment cascade, it may be useful in inhibiting inflammatory angiogenesis, which is related to angiogenesis associated with tumor growth and metastasis, corneal neovascularization, and CNV.

The present invention also relates, in part, to the discovery that VAP-1 blockade inhibits lymphangiogencsis in animals, for example, animals exhibiting corneal lymphangiogcncsis. Accordingly, the invention describes methods and compositions for treating lymphangiogenic conditions by administering a VAP-1 inhibitor to a subject in an amount sufficient to inhibit lymphangiogenesis. Inhibition of lymphangiogenesis using a VAP-1 inhibitor can include lymph vessel regression and/or inhibition of lymph vessel formation. Inhibition of new lymph vessel formation includes cessation of new lymph formation and/or a decrease in the rate of new lymph vessel formation, for example, as compared to an untreated control.

Lymphatic vessels and their formation (lymphangiogenesis) are implicated in a number of pathological conditions, such as neoplasm, metastasis, organization of immunologically active lymphocytic infiltrates following organ transplantation, edema, rheumatoid arthritis, psoriasis, and wound healing. Lymphangiogenesis has been shown to be induced by certain growth factors, by inflammation, and/or by tumor growth. Lymphangiogenesis has been shown to be induced by VEGF activation of VEGF receptor 3, and in some instances, VEGF receptor 2.

VAP-1 inhibitors include, for example, a protein such as an antibody specific for VAP-1 and/or the conjugate binding partner of VAP-1, and/or fragments thereof, as described more fully below. VAP-1 inhibitors also include nucleic acids and small molecules as described more fully below. VAP-1 has been shown to regulate leukocyte recruitment under physiological and pathological conditions, both as an adhesion molecule and as an enzyme. Membrane-bound VAP-1 has been shown to mediate the interaction between leukocytes and activated endothelial cells in inflamed vessels. Both the direct adhesive and enzymatic functions of VAP-1 are believed to be involved in the leukocyte recruitment cascade. Previous studies have revealed that VAP-1 is identical with the cell-surface enzyme, semicarbazide-sensitive amine oxidase (SSAO), which catalyzes the deamination of primary amines, such as methylamine and aminoacetone. This reaction generates toxic formaldehyde and methylglyoxal, hydrogen peroxide and ammonia, which are known as reactive chemicals and major reactive oxygen species. Previously, SSAO activity has been detected in retinal tissues in connection with vascular permeability. Accordingly, VAP-1 inhibitors have been investigated in connection with vascular hyperpermeable diseases and inflammatory conditions.

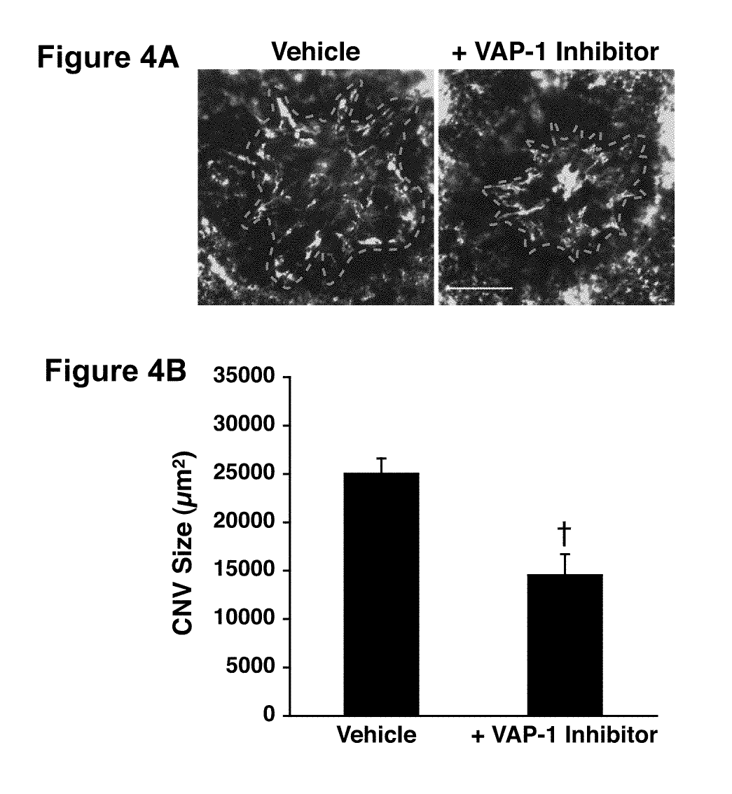

As noted above, the present invention relates, in part, to the discoveries that VAP-1 plays a role in angiogenesis and that VAP-1 blockade inhibits angiogenesis in animal models, for example, animal models of CNV and corneal angiogenesis. For example, the Examples below indicate that VAP-1 plays a role in CNV, an integral component of AMD, and in corneal angiogenesis. In the CNV model of Example 1, VAP-1 blockade significantly reduced CNV size seven days after laser-injury induction of CNV (see, for example, FIGS. 4A and 4B). In the corneal angiogenesis model of Example 2, the use of a VAP-1 inhibitor was shown to significantly inhibit corneal angiogenesis in animals treated with the VAP-1 inhibitor as compared to animals that did not receive the VAP-1 inhibitor.

Inhibition of angiogenesis includes blood vessel regression and/or inhibition of blood vessel formation. For example, FIG. 4A shows two areas of angiogenesis due to CNV that are surrounded by dotted lines. In untreated animals, laser injury causes a large lesion indicative of new blood vessel formation (FIG. 4A left, vehicle). The lesion size is much smaller with the use of a VAP-1 inhibitor (FIG. 4A right, +VAP-1 Inhibitor). In this model, there are two ways of achieving the beneficial effects of an inhibitor. First, growth of the blood vessels may be impeded. Second, new blood vessels may regress.

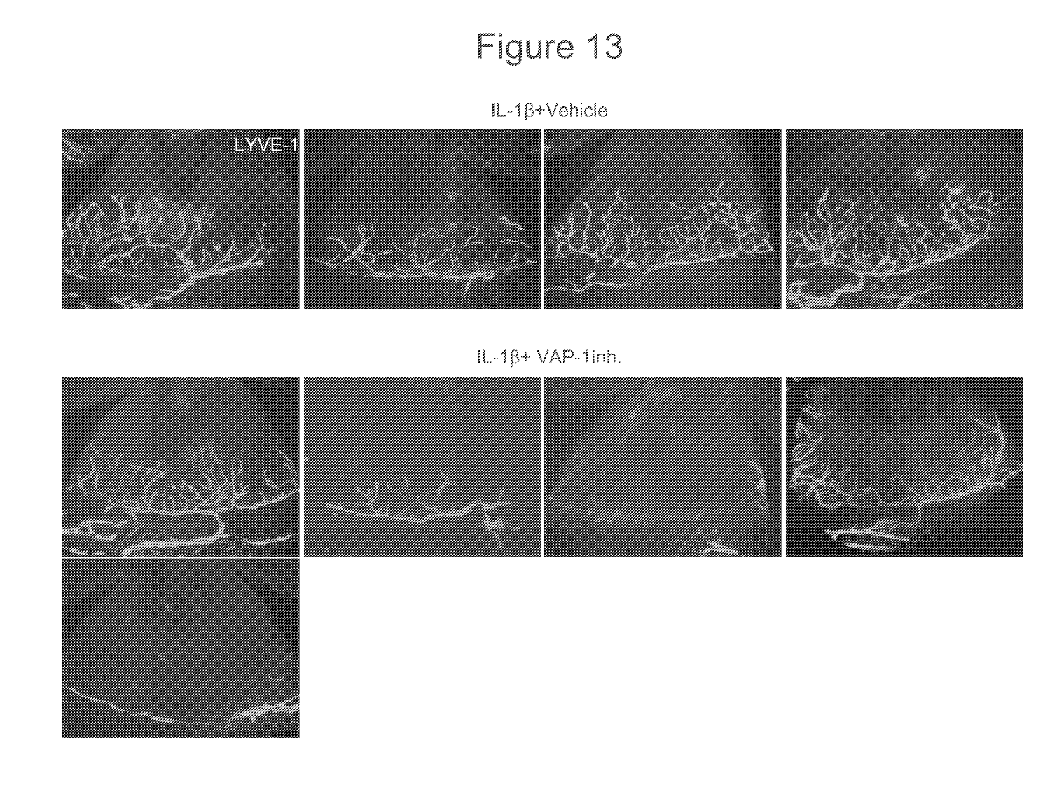

The present invention also relates, in part, to the discovery that VAP-1 blockade inhibits lymphangiogenesis in animal models, for example, animal models of corneal lymphangiogenesis. For example, in the corneal lymphangiogenesis model of Example 2, the use of a VAP-1 inhibitor was shown to inhibit corneal lymphangiogenesis in animals treated with the VAP-1 inhibitor as compared to animals that did not receive the VAP-1 inhibitor. Inhibition of lymphangiogenesis includes lymph vessel regression and/or inhibition of lymph vessel formation. For example, FIG. 13 compares lymph vessels in animals treated with VAP-1 inhibitor to untreated animals, following induction of lymphangiogenesis with an IL-1β pellet. More lymph vessels appear in the untreated animals, indicative of new lymph vessel formation (FIG. 13, IL-1β+vehicle) than in animals treated with a VAP-1 inhibitor (FIG. 13, IL-1β+VAP-1 inhibitor). In this model, there are two ways of achieving the beneficial effects of an inhibitor. First, growth of the lymph vessels may be impeded. Second, new lymph vessels may regress.

### I. Indications of VAP-1 Inhibition

The present invention includes methods and compositions for treating angiogenic conditions by administering a VAP-1 inhibitor to a subject in an amount sufficient to inhibit angiogenesis. The angiogenic conditions that may treated with the methods of this invention include cancer, diabetes, diabetic retinopathy, age-related macular degeneration, rheumatoid arthritis, psoriasis, complications of AIDS (Kaposi's sarcoma), Alzheimer's disease, chronic inflammatory diseases (e.g. Crohn's disease and ulcerative colitis), acute inflammation, rheumatic diseases, autoimmune diseases, systemic inflammatory diseases including systemic lupus erythematosus (SLE), systemic sclerosis (SSc), Sjögren's syndrome (SS), mixed connective tissue disease (MCTD), polymyositis/dermatomyositis (PM/DM) and systemic vasculitis, endometriosis, skin diseases (e.g. psoriasis), thrombotic diseases (including diseases related to platelet function), and/or diseases related to coagulation and complement cascade. Particularly, the condition may be cancer, an ocular angiogenic condition such as unwanted choroidal neovasculature or corneal angiogenesis, scar formation, tissue repair, wound healing, atherosclerosis, and/or arthritis. Moreover, the VAP-1 inhibitor can be administered to a subject in an amount sufficient to inhibit angiogenesis related to physiologic aging and/or a condition related to aging.

The present invention also includes methods and compositions for treating lymphangiogenic conditions by administering a VAP-1 inhibitor to a subject in an amount sufficient to inhibit lymphangiogenesis. The lymphangiogenic conditions include, for example, cancer, neoplasm, metastasis, organ transplantation, particularly the organization of immunologically active lymphocytic infiltrates following organ transplantation, edema, rheumatoid arthritis, scar formation, tissue repair, psoriasis, and wound healing. Particularly, the condition may include cancer or an ocular lymphangiogenic condition such as corneal lymphangiogenesis. Moreover, the VAP-1 inhibitor can be administered to a subject in an amount sufficient to inhibit lymphangiogenesis related to physiologic aging and/or a condition related to aging.

a. Inhibition of VAP-1 as a Treatment for Cancer

The invention provides methods for treating cancer, the second most common cause of death in Western societies. In one aspect, the methods include administering a VAP-1 inhibitor to a subject in an amount sufficient to inhibit angiogenesis. In certain embodiments, the angiogenesis inhibition attenuates tumor growth and/or inhibits tumor metastasis. In another aspect, the methods include administering a VAP-1 inhibitor to a subject in an amount sufficient to inhibit lymphangiogenesis. In certain embodiments, the lymphangiogenesis inhibition attenuates tumor growth and/or inhibits tumor metastasis.

Cancer is characterized by cells that divide in an uncontrolled fashion. Most organs can be the primary source of cancer. However, the most common sites are lung, breast and prostate. Cancer cells frequently aggregate as tumors, a mass of rapidly dividing and growing cancer cells. The rapidly growing cancer cells within a tumor requires a large influx of oxygen and other essential nutrients and a means to expel waste. However, tumors often have no pre-established vessels to meet these needs.

Tumors induce vessel growth by secreting various growth factors such as VEGF and bFGF. These factors induce vessel growth into the tumor, which supplies the required nutrients and expulsion of waste, and thereby allows for rapid tumor expansion. Certain cancer cells have been shown to facilitate angiogenesis by stopping the production of an anti-VEGF enzyme, PKG, which shifts the equilibrium of blood vessel growth toward angiogenesis. Angiogenesis also can facilitate cancer metastasis. Many cancers metastasize to other sites in the organism. The ensuing secondary growth of the tumor masses is then the primary health hazard in cancer patients. It is believed that cancer cells can spread within the body by different mechanisms. In order for cancer to metastasize, individual cancer cells typically leave a tumor by entering a vessel and migrating to another site within the body. Accordingly, in the absence of established vessels to the tumor, it is difficult for individual cells to migrate away from the tumor.

It has been found that some blood vessels within a tumor are comprised of a mosaic of both endothelial cells and cancerous cells, which allows for cell migration of the cancerous cells directly into the bloodstream. Alternatively, cancer may spread through the lymphatic system to distant sites in the body. Another mode of metastasis can be through direct invasion into the surrounding tissues.

Accordingly, anti-angiogenesis and anti-lymphangiogenesis factors that inhibit the vascularization of a tumor have been investigated as means for controlling cancer cell growth and metastasis. For example, anti-angiogenesis factors such as angiostatin, endostatin, tumstatin, and the anti-VEGF antibody AVASTIN® have been investigated as compounds to inhibit neovascularization of tumors. Endothelial cells are a particularly appealing target for inhibiting vessel growth to tumors because they are more stable than cancer cells, which can mutate and become resistant to treatment. However, endothclial cells growing within tumors have been shown to display genetic abnormalities, which suggests that vessels growing within tumors may also be capable of mutation and resistance. Accordingly, new mechanisms for inhibition of angiogenesis and for inhibition of lymphangiogenesis, such as treatment with a VAP-1 inhibitor, may be critical to a regimen of treatment directed at depriving a tumor of new vessel growth and/or to facilitate the regression of tumor vessels. In addition, since VAP-1 actively modulates leukocyte-endothelial cell interaction in both physiological and pathological conditions, it may be particularly useful in cancer of hematological cells and/or immune cells. There are two mechanisms by which VAP-1 inhibition may be beneficial in such conditions. First, it may inhibit release of leukemic cells from the bone marrow or other sources of origin. Second, it may inhibit recruitment of the cells in various vascular beds in the body, reducing tissue injury and leukostasis in capillaries.

It is understood that the administration of a VAP-1 inhibitor to inhibit angiogenesis as described herein can be part of a combination therapy, for example, administered with (e.g. before, during, or after) administration of any of the anti-angiogenesis factors and/or anti-lymphangiogenesis factors described above, chemotherapy treatment, and/or radiation treatment. Further, it is understood that the administration of a VAP-1 inhibitor to inhibit lymphangiogenesis as described herein can be part of a combination therapy, for example, administered with (e.g. before, during, or after) administration of any of the anti-angiogenesis factors and/or anti-lymphangiogenesis factors described above, chemotherapy treatment, and/or radiation treatment.

b. Inhibition of VAP-1 as a Treatment for Ocular Angiogenesis

The invention provides an improved method for treating ocular disorders associated with unwanted ocular angiogenesis, for example, disorders associated with corneal angiogenesis and/or CNV. The method includes administering to the subject an amount of a VAP-1 inhibitor that is sufficient to inhibit angiogenesis, for example, corneal angiogenesis and/or CNV. The VAP-1 inhibitor is administered in an amount sufficient to regress blood vessels or inhibit blood vessel formation in one or more regions and/or structures of the eye.

The invention also provides an improved method for treating ocular disorders associated with unwanted ocular lymphangiogenesis, for example, disorders associated with corneal lymphangiogenesis. The method includes administering to the subject an amount of a VAP-1 inhibitor that is sufficient to inhibit lymphangiogenesis, for example, corneal lymphangiogenesis. The VAP-1 inhibitor is administered in an amount sufficient to regress blood vessels or inhibit lymph vessel formation in one or more regions and/or structures of the eye.

Ocular angiogenesis refers to blood vessel growth within a structure of the eye, for example, the cornea or the choroid. Ocular lymphangiogenesis refers to lymph vessel growth within a structure of the eye, for example, the cornea. The cornea is the transparent front part of the eye. It is normally devoid of both blood and lymphatic vessels and, therefore, is described as being both immune privileged and angiogenic privileged. New vessel growth to the cornea is associated with a state of disease secondary to a variety of corneal insults, including contact lens use. Contact lens use commonly induces superficial new vessel growth rather than new vessel growth, for example, by deep stromal vessels. However, both superficial and serious vessel growth have been reported with use of hydrogel, polymethyl methacrylate, and rigid gas permeable contact lenses, particularly with extended wear use contact lenses.

Deep stromal new vessel growth to the cornea indicates a profound insult, for example hypoxia, and can lead to loss of optical transparency of the cornea through, for example, stromal hemorrhage, scarring, and lipid deposition. Corneal new vessel growth is believed to result from an inflammatory or hypoxic disruption, for example, by the contact lens either mechanically irritating the limbal sulcus or creating corneal hypoxia to stimulate limbal inflammation, epithelial erosion, or hypertrophy. Ocular angiogenesis and ocular lymphangiogenesis have also been observed in connection with corneal transplants.

These insults can stimulate production of angiogenic factors by local epithelial cells, keratocytes, and infiltrating leukocytes, for example, macrophages and neutrophils. Such angiogenic factors may include acidic and basic fibroblast growth factors, interleukin 1 (IL-1), and vascular endothelial growth factor (VEGF), and may stimulate a localized enzymatic degradation of the basement membrane of perilimbal vessels at the apex of a vascular loop, thereby inducing vascular endothelial cell migration and proliferation to form new blood vessels.

Choroidal angiogenesis, also referred to herein as choroidal neovascularization or CNV, is associated with conditions that include, for example, neovascular AMD, ocular histoplasmosis syndrome, pathologic myopia, angioid streaks, idiopathic disorders, choroiditis, choroidal rupture, overlying choroid nevi, and certain inflammatory diseases. Choroidal ncovascularization (CNV) is the main cause of severe vision loss in patients with age-related macular degeneration (AMD). There is evidence that inflammatory cells are critically involved in the formation of CNV lesions and play a role in the pathogenesis of age-related macular degeneration. Inflammatory cells have been found in CNV lesions that were surgically excised from AMD patients and in autopsy eyes with CNV. In particular, macrophages have been implicated in the pathogenesis of AMD due to their spatiotemporal distribution in the proximity of the CNV lesion both in humans and experimental models.

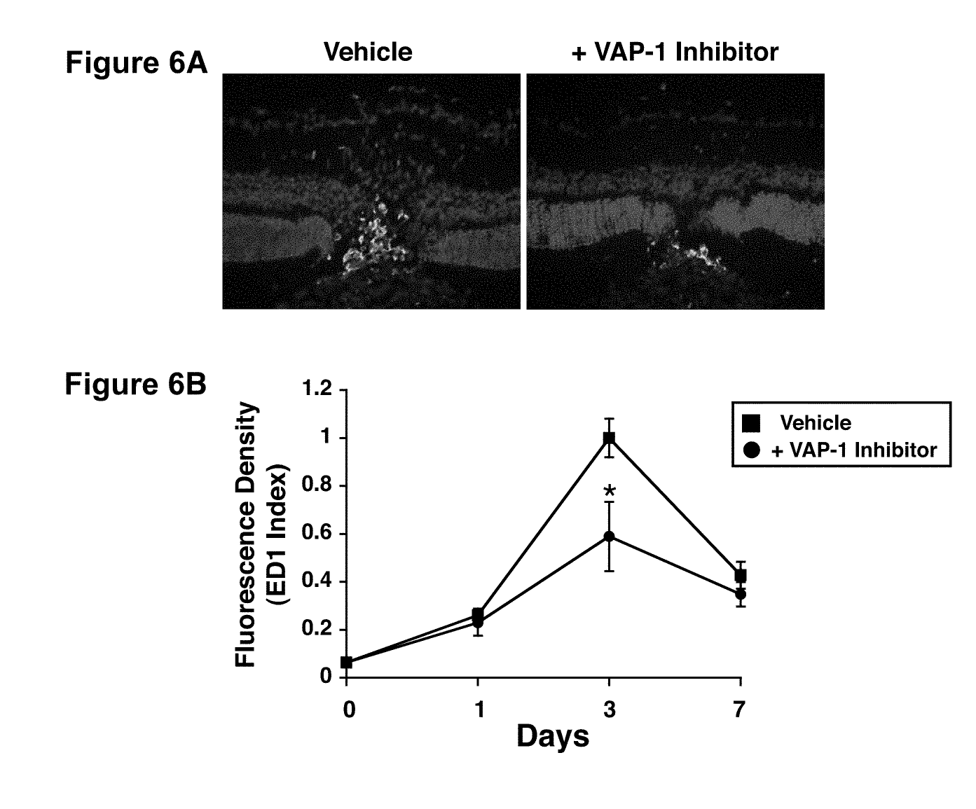

Macrophages are known to be a source of proangiogenic and inflammatory cytokines, such as vascular endothelial growth factor (VEGF) and tumor necrosis factor (TNF)-α, both of which significantly contribute to the pathogenesis of CNV. Most of the macrophages found in the proximity of the laser-induced CNV lesions following PDT likely are derived from newly recruited peripheral blood monocytes and not resident macrophages. As shown in Example 1 below, VAP-1 inhibition reduces both CNV and the presence of macrophages at the height of CNV formation in a CNV animal model. See, for example, FIGS. 6A and 6B.

### II. Exemplary VAP-1 Inhibitors

The term “VAP-1 inhibitor” is understood to mean any molecule, for example, a protein, peptide, nucleic acid (ribonucleic acid (RNA) or deoxyribonucleic acid (DNA)), peptidyl nucleic acid, small molecule (organic compound or inorganic compound), that inhibits angiogenesis (e.g. regresses a blood vessel and/or inhibits blood vessel formation) in a subject. The term “VAP-1 inhibitor” is also understood to mean any molecule, for example, a protein, peptide, nucleic acid (ribonucleic acid (RNA) or deoxyribonucleic acid (DNA)), peptidyl nucleic acid, small molecule (organic compound or inorganic compound), that inhibits lymphangiogenesis (e.g. regresses a lymph vessel and/or inhibits lymph vessel formation) in a subject. Accordingly, an “effective amount” of a VAP-1 inhibitor is an amount of a VAP-1 inhibitor sufficient to inhibit angiogenesis and/or lymphangiogcncsis.

A variety of VAP-1 inhibitors may be used in the invention. Useful VAP-1 inhibitors, include but are not limited to, for example, anti-VAP-1 neutralizing antibody (available, for example, from R&D Systems, Minneapolis, Minn., catalogue nos. AF3957, MAB39571, and MAB3957; Everest Biotech, Oxford, United Kingdom, catalogue no. EB07582; and antibodies identified in U.S. Pat. Nos. 4,704,692; 6,066,321 and 5,580,780 and Koskinen et al. (2004) BLOOD 103:3388: Arvilommi et al. (1996) EUR. J. IMMUNOL. 26:825, Salmi et al. (1993) J. EXP. MED., 178:2255, and Kirten et al. (2005) EUR. J. IMMUNOL. 35:3119); small molecules such as phenylhydrazine, 5-hydroxytryptamine, 3-bromopropylamine, N-(phenyl-allyl)-hydrazine HCl (LJP-1207), 2-hydrazinopyridine, MDL-72274 ((E)-2-phenyl-3-chloroallylamine hydrochloride), MDL-72214 (2-phenylallylamine), mexiletine, isoniazid, imipramine, maprotiline, zimeldine, nomifensine, azoprocarbazine, monomethylhydrazine, d1-alpha methyltryptamine, d1-alpha methylbenzylamine, MD780236 (Dostert et al. (1984), J. PHARMACY & PHARMACOL., 36:782), 2-(dimethyl(2-phenylethyl)silyl) methanamine, cuprozine, alkylamino derivatives of 4-amniomethylpyridine (Bertini et al. (2005) J. MED. CHEM. 48:664), kynuramine, those identified in PCT Publication Nos. WO 2004/087138 (nationalized in the United States as U.S. Published Application No. 2006/0229346), WO 2004/067521. WO2005/014530, and WO 2005/089755, in U.S. Published Application Nos. 2004/0236108, 2004/0259923, 2005/0096360, and 2006/0025438, and in U.S. Pat. Nos. 7,125,901 and 6,624,202, and small molecules that bind VAP-1 to prevent or reduce its binding to its cognate receptor or ligand; peptides (for example, the peptide inhibitors discussed in Yegutkin et al. (2004) EUR. J. IMMUNOL. 34:2276 and Wang et al. (2006) J. MED. CHEM. 49:2166); nucleic acids (for example, anti-VAP-1 aptamers and siRNAs identified in PCT Publication No. WO2006/134203); certain antibodies, antigen binding fragments thereof, and peptides that bind preferentially to VAP-1 or the VAP-1 cognate receptor or ligand; antisense nucleotides and double stranded RNA for RNAi that ultimately reduce or eliminate the production of either VAP-1 or its cognate receptor or ligand; soluble VAP-1; and/or soluble VAP-1 cognate receptor or ligand. These VAP-1 inhibitors can act as direct or indirect inhibitors of angiogenesis and/or lymphangiogenesis.

a. Exemplary VAP-1 Inhibitors-Proteins

Antibodies (e.g., monoclonal or polyclonal antibodies) having sufficiently high binding specificity for the marker or target protein (for example, VAP-1 or its cognate receptor or ligand) can be used as VAP-1 inhibitors. As noted above, the term “antibody” is understood to mean an intact antibody (for example, a monoclonal or polyclonal antibody); an antigen binding fragment thereof, for example, an Fv, Fab, Fab′ or (Fab′)₂ fragment; or a biosynthetic antibody binding site, for example, an sFv, as described in U.S. Pat. Nos. 5,091,513; 5,132,405; 5,258,498; and U.S. Pat. Nos. 5,482,858; and 4,704,692. A binding moiety, for example, an antibody, is understood to bind specifically to the target, for example, VAP-1 or its receptor, when the binding moiety has a binding affinity for the target greater than about 10⁵ M⁻¹, more preferably greater than about 10⁷ M⁻¹.

Antibodies against VAP-1 or its receptor may be generated using standard immunological procedures well known and described in the art. See, for example, Practical Immunology, Butt, N. R., ed., Marcel Dekker, NY, 1984. Briefly, isolated VAP-1 or its ligand or receptor is used to raise antibodies in a xenogeneic host, such as a mouse, goat or other suitable mammal. The VAP-1 or its ligand or receptor is combined with a suitable adjuvant capable of enhancing antibody production in the host, and injected into the host, for example, by intraperitoneal administration. Any adjuvant suitable for stimulating the host's immune response may be used. A commonly used adjuvant is Freund's complete adjuvant (an emulsion comprising killed and dried microbial cells). Where multiple antigen injections are desired, the subsequent injections may comprise the antigen in combination with an incomplete adjuvant (for example, a cell-free emulsion).

Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target protein and have the desired binding affinity.

Antibody binding domains also may be produced biosynthetically and the amino acid sequence of the binding domain manipulated to enhance binding affinity with a preferred epitope on the target protein. Specific antibody methodologies are well understood and described in the literature. A more detailed description of their preparation can be found, for example, in Practical Immunology, Butt, W. R., ed., Marcel Dekker, New York. 1984.

Other proteins and peptides also can be used as a VAP-1 inhibitor. Proteins and peptides of the invention can be produced in various ways using approaches known in the art. For example, DNA molecules encoding the protein or peptide of interest are chemically synthesized, using a commercial synthesizer and known sequence information. Such synthetic DNA molecules can be ligated to other appropriate nucleotide sequences, including, e.g., expression control sequences, to produce conventional gene expression constructs encoding the desired proteins and peptides. Production of defined gene constructs is within routine skill in the art.

The nucleic acids encoding the desired proteins and peptides can be introduced (ligated) into expression vectors, which can be introduced into a host cell via standard transfection or transformation techniques known in the art. Exemplary host cells include, for example, E. coli cells. Chinese hamster ovary (CHO) cells, HeLa cells, baby hamster kidney (BHK) cells, monkey kidney cells (COS), human hepatocellular carcinoma cells (e.g., Hep G2), and myeloma cells that do not otherwise produce immunoglobulin protein. Transfected host cells can be grown under conditions that permit the host cells to express the genes of interest, for example, the genes that encode the proteins or peptides of interest. The resulting expression products can be harvested using techniques known in the art.

The particular expression and purification conditions will vary depending upon what expression system is employed. For example, if the gene is to be expressed in E. coli, it is first cloned into an expression vector. This is accomplished by positioning the engineered gene downstream from a suitable bacterial promoter, e.g., Trp or Tac, and a signal sequence, e.g., a sequence encoding fragment B of protein A (FB). The resulting expressed fusion protein typically accumulates in refractile or inclusion bodies in the cytoplasm of the cells, and may be harvested after disruption of the cells by French press or sonication. The refractile bodies then are solubilized, and the expressed proteins refolded and cleaved by the methods already established for many other recombinant proteins.

If the engineered gene is to be expressed in eukaryotic host cells, for example, myeloma cells or CHO cells, it is first inserted into an expression vector containing a suitable eukaryotic promoter, a secretion signal, and various introns. The gene construct can be transfected into myeloma cells or CHO cells using established transfection protocols. Such transfected cells can express the proteins or peptides of interest, which may be attached to a protein domain having another function.

Protein treatment agents, such as antibodies and exogenous proteins, are known in the art. For example, VAP-1 inhibitors include, but are not limited to, for example, anti-VAP-1 neutralizing antibody (available, for example, from R&D Systems, Minneapolis, Minn., catalogue nos. AF3957, MAB39571, and MAB3957; Everest Biotech, Oxford, United Kingdom, catalogue no. EB07582; and antibodies identified in U.S. Pat. Nos. 4,704,692; 6,066,321 and 5,580,780 and Koskinen et al. (2004) BLOOD 103:3388; Arvilommi et al. (1996) EUR. J. IMMUNOL. 26:825, Salmi et al. (1993) J. EXP. MED., 178:2255, and Kirten et al. (2005) EUR. J. IMMUNOL. 35:3119); and peptides (for example, the peptide inhibitors discussed in Yegutkin et al. (2004) EUR. J. IMMUNOL. 34:2276 and Wang et al. (2006) J. MED. CHEM. 49:2166).

b. Exemplary VAP-1 Inhibitors—Nucleic Acids

To the extent that the VAP-1 inhibitor is a nucleic acid or peptidyl nucleic acid, such compounds may be synthesized by any of the known chemical oligonucleotide and peptidyl nucleic acid synthesis methodologies known in the art (see, for example, PCT/EP92/20702 and PCT/US94/013523) and used in antisense therapy. Anti-sense oligonucleotide and peptidyl nucleic acid sequences, usually 10 to 100 and more preferably 15 to 50 units in length, are capable of hybridizing to a gene and/or mRNA transcript and, therefore, may be used to inhibit transcription and/or translation of a target protein.

VAP-1 gene expression can be inhibited by using nucleotide sequences complementary to a regulatory region of the VAP-1 gene (e.g., the VAP-1 promoter and/or a enhancer) to form triple helical structures that prevent transcription of the VAP-1 gene in target cells. See generally, Helene (1991) ANTICANCER DRUG DES. 6(6): 569-84, Helene et al. (1992) ANN. NY ACAD. SCI. 660: 27-36; and Maher (1992) BIOESSAYS 14(12): 807-15. The antisense sequences may be modified at a base moiety, sugar moiety or phosphate backbone to improve, e.g., the stability, hybridization, or solubility of the molecule. For example, in the case of nucleotide sequences, phosphodiester linkages may be replaced by thioester linkages making the resulting molecules more resistant to nuclease degradation. Alternatively, the deoxyribose phosphate backbone of the nucleic acid molecules can be modified to generate peptide nucleic acids (see Hyrup et al. (1996) BIOORG. MED. CHEM. 4(1): 5-23). Peptidyl nucleic acids have been shown to hybridize specifically to DNA and RNA under conditions of low ionic strength. Furthermore, it is appreciated that the peptidyl nucleic acid sequences, unlike regular nucleic acid sequences, are not susceptible to nuclease degradation and, therefore, are likely to have greater longevity in vivo. Furthermore, it has been found that peptidyl nucleic acid sequences bind complementary single stranded DNA and RNA strands more strongly than corresponding DNA sequences (PCT/EP92/20702). Similarly, oligoribonucleotide sequences generally are more susceptible to enzymatic attack by ribonucleases than are deoxyribonucleotide sequences, such that oligodeoxyribonucleotides are likely to have greater longevity than oligoribonucleotides for in vivo use.

Additionally, RNAi can serve as a VAP-1 inhibitor. To the extent RNAi is used, double stranded RNA (dsRNA) having one strand identical (or substantially identical) to the target mRNA (e.g. VAP-1 mRNA) sequence is introduced to a cell. The dsRNA is cleaved into small interfering RNAs (siRNAs) in the cell, and the siRNAs interact with the RNA induced silencing complex to degrade the target mRNA, ultimately destroying production of a desired protein (e.g., VAP-1). Alternatively, the siRNA can be introduced directly. Examples of siRNAs suitable for targeting VAP-1 are described, for example, in PCT Publication No. WO 2006/134203.

Additionally, an aptamer can be used as a VAP-1 inhibitor and may target VAP-1. Methods for identifying suitable aptamers, for example, via systemic evolution of ligands by exponential enrichment (SELEX), are known in the art and are described, for example, in Ruckman et al. (1998) J. Biol. Chem. 273: 20556-20567 and Costantino et al. (1998) J. Pharm. Sci. 87: 1412-1420. c. Exemplary VAP-1 inhibitors—small molecules

To the extent that the VAP-1 inhibitor is a small molecule, either an organic or inorganic compound, such compounds may be synthesized, extracted and/or purified by standard procedures known in the art. Many small molecule VAP-1 inhibitors are known, for example, as described in PCT Publication Nos. WO 2004/087138 (nationalized in the United States as U.S. Published Application No. 2006/0229346). WO 2004/067521, WO 2005/014530 and WO 2005/089755 and in U.S. Pat. Nos. 7,125,901 and 6,624,202. The common structural features of these known small molecule VAP-1 inhibitors can be used to identify additional small molecules that can be used as VAP-1 inhibitors. Accordingly, VAP-1 inhibitors of the present invention include thiazole and derivatives thereof, many of which are published, for example, in PCT Publication No. WO 2004/067521 and in U.S. Published Application Nos. 2004/0236108, 2004/0259923, 2005/0096360, and 2006/0025438 and also in U.S. Pat. No. 7,125,901. VAP-1 inhibitors of the present invention also include hydrazine compounds and derivatives thereof, many of which are published, for example, in U.S. Pat. No. 6,624,202 and in U.S. Published Application Nos. 2002/0173521, 2002/0198189, 2003/0125360 and 2004/0106654.

For example, a VAP-1 inhibitor can have the general structure of formula (I) (hereinafter sometimes referred to as Compound (I)):

R¹—NH—X—Y—Z (I).

In formula (1), R¹ may be an acyl; X may be a bivalent residue derived from optionally substituted thiazole; Y may be a bond, lower alkylene, lower alkenylene or —CONH—; and Z may be a group of the formula:

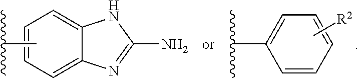

R² may be a group of the formula: -A-B-D-E wherein A may be a bond, lower alkylene, —NH— or —SO₂—; B may be a bond, lower alkylene, —CO— or —O—; D may be a bond, lower alkylene, —NH— or —CH₂NH—; and E optionally may be protected amino, —N═CH₂,

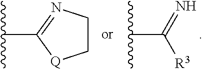

Q may be —S— or —NH—; and R³ may be hydrogen, lower alkyl, lower alkylthio or —NH—R⁴ wherein R⁴ may be hydrogen, —NH₂ or lower alkyl; or a derivative thereof; or a pharmaceutically acceptable salt thereof.

In certain embodiments of formula (I), Z may be a group of the formula:

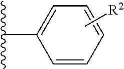

wherein R₂ may be a group of the formula:

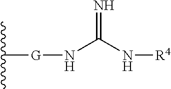

(wherein G may be a bond, —NHCOCH₂— or lower alkylene and R⁴ may be hydrogen, —NH₂ or lower alkyl); —NH₂; —CH₂NH₂; —CH₂ONH₂; —CH₂ON═CH₂;

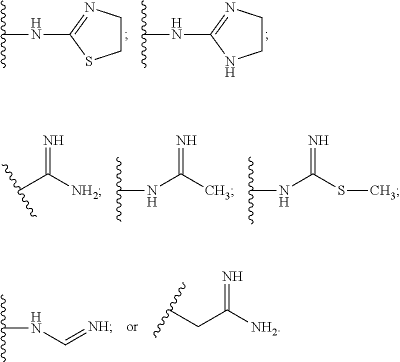

In certain embodiments of formula (I), R¹ may be alkylcarbonyl and X may be a bivalent residue derived from thiazole optionally substituted by methylsulfonylbenzyl. In certain embodiments of formula (I), X is represented by:

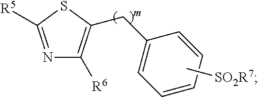

wherein, R⁵ is a bond to NH, R⁶ is a bond to Y, R⁷ is C₁-C₆ alkyl, and m is 1, 2, or 3.

Specific examples of small molecule VAP-1 inhibitors include:
N-{4-[2-(4-{[amino (imino) methyl] amino} phenyl) ethyl]-1,3-thiazol-2-yl} acetamide;
N-[4-(2-{4-[(aminooxy)methyl]phenyl}ethyl)-1,3-thiazol-2-yl] acetamide;
N-{4-[2-(4-{[amino (imino) methyl] amino} phenyl) ethyl]-5-[4-(methylsulfonyl) benzyl]-1,3-thiazol-2-yl} acetamide;
N-{4-[2-(4-{[hydrazino (imino) methyl] amino} phenyl) ethyl]-5-[4-(methylsulfonyl) benzyl]-1,3-thiazol-2-yl} acetamide;
N-{4-[2-(4-{[hydrazino (imino) methyl] amino} phenyl) ethyl]-1,3-thiazol-2-yl} acetamide;
N-(4-{2-[4-(2-{[amino (imino) methyl] amino} ethyl) phenyl] ethyl}-1,3-thiazol-2-yl) acetamide; and derivatives thereof, or pharmaceutically acceptable salts thereof.

Additionally, a small molecule VAP-1 inhibitor can have the structure of formula (II) (hereinafter sometimes referred to as Compound (II))

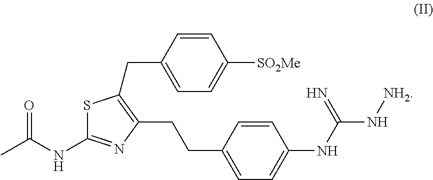 [SMILES: CC(=O)Nc1nc(CCc2ccc(NC(=N)NN)cc2)c(Cc2ccc(S(C)(=O)=O)cc2)s1]

This compound was used in Examples 1 and 2, below.

Further examples of small molecule VAP-1 inhibitors include hydrazine compounds, as described in U.S. Pat. No. 6,624,202, having the structure of formula (III) or (IV).

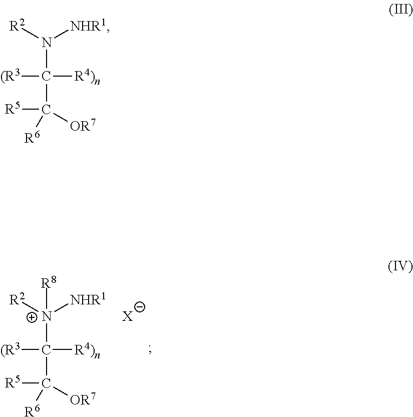

or a stereoisomer or pharmaceutically acceptable solvate, hydrate, or salt thereof.

In formula (III) or (IV) R¹ can be hydrogen, (C₁-C₄)alkyl, aralkyl, (C₂-C₅)alkanoyl, aroyl or heteroaroyl; R² can be hydrogen, or optionally substituted (C₁-C₄)alkyl, optionally substituted cycloalkyl or optionally substituted aralkyl: R³-R⁶, which can be the same or different, can be hydrogen, optionally substituted (C₁-C₄)alkyl, optionally substituted aralkyl, optionally substituted phenyl or optionally substituted heteroaryl; or R¹ and R², together with the atoms to which they are attached, can represent an optionally substituted heterocycle, or R² and R³, together with the atoms to which they are attached, can represent an optionally substituted heterocycle, or R³ and R⁵, together with the atoms to which they are attached, can represent a saturated, optionally substituted carbocycle; R⁷ can be hydrogen, (C₁-C₄)alkyl, (C₂-C₅)alkanoyl or aralkyl; R⁸ can be (C₁-C₄)alkyl or aralkyl; n can be 1, 2 or 3; and X can be chloride, bromide, iodide or R²-sulfate, where R² is as defined above with respect to formulas (III) and (IV).

Further examples of small molecule VAP-1 inhibitors are described in PCT Publication Nos. WO 2004/087138 (nationalized in the United States as U.S. Published Application No. 2006/0229346), WO 2004/067521, WO 2005/014530 and WO 2005/089755, in U.S. Published Application Nos. 2004/0236108, 2004/0259923, 2005/0096360, and 2006/0025438 and in U.S. Pat. Nos. 7,125,901 and 6,624,202 and also include molecules such as phenylhydrazine, 5-hydroxytryptamine, 3-bromopropylamine, N-(phenyl-allyl)-hydrazine HCl (LJP-1207), 2-hydrazinopyridine, MDL-72274 ((E)-2-phenyl-3-chloroallylamine hydrochloride), MDL-72214 (2-phenylallylamine), mexiletine, isoniazid, imipramine, maprotiline, zimeldine, nomifensine, azoprocarbazine, monomethylhydrazine, d1-alpha methyltryptamine, d1-alpha methylbenzylamine, MD780236 (Dostert et al. (1984), J. PHARMACY & PHARMACOL., 36:782), 2-(dimethyl(2-phenylethyl)silyl) methanamine, cuprozine, alkylamino derivatives of 4-amniomethylpyridine (Bertini et al. (2005) J. MED. CHEM. 48:664), and kynuramine.

### III. VAP-1 Inhibition as a Combination Therapy

It is contemplated that a variety of VAP-1 inhibitors may be combined with other treatments for treating unwanted vasculature, such as blood vessels and/or lymphatic vessels. For example, a VAP-1 inhibitor may be administered with (e.g. before, during, or after administration of) any of the anti-angiogenesis and/or anti-lymphangiogenesis factors described herein, chemotherapy treatment, radiation treatment, PDT therapy, treatment to modulate VEGF, and/or treatment to modulate apoptosis. Such combination therapy may be used to treat any condition associated with angiogenesis, including cancer and an ocular angiogenic condition such as corneal angiogenesis and unwanted CNV. Combination therapy may also be used to treat any condition associated with lymphangiogenesis, for example, cancer or an ocular lymphangiogenic condition such as corneal lymphangiogenesis.

The VAP-1 inhibitor may be administered with (e.g. before, during, or after) a factor that inhibits one or more known endogenous angiogenic factors, which also may be indirectly inhibited by a VAP-1 inhibitor, including angiogenin, angiopoietin-1, Del-1, fibroblast growth factors: acidic (aFGF) and basic (bFGF), follistatin, granulocyte colony-stimulating factor (G-CSF), hepatocyte growth factor (HGF)/scatter factor (SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), and vascular endothelial growth factor (VEGF)/vascular permeability factor (VPF).

The VAP-1 inhibitor also may be administered with one or more known endogenous angiogenesis inhibitors, including angioarrestin, angiostatin (plasminogen fragment), antiangiogenic antithrombin III, cartilage-derived inhibitor (CDI), CD59 complement fragment, endostatin (collagen XVIII fragment), fibronectin fragment, Gro-beta, heparinases, heparin hexasaccharide fragment, human chorionic gonadotropin (hCG), interferon alpha/betaigamma, interferon inducible protein (IP-10), Interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), retinoids, tetrahydrocortisol-S, thrombospondin-1 (TSP-1), transforming growth factor-beta (TGF-b), vasculostatin, and vasostatin (calreticulin fragment).

The VAP-1 inhibitor also may be administered with one or more known chemotherapeutic agents (antineoplastic agent) including alkylating agents, antimetabolites, natural products and their derivatives, hormones and steroids (including synthetic analogs), and synthetics. Examples of compounds within these classes include alkylating agents (including nitrogen mustards, ethylenimine derivatives, alkyl sulfonates, nitrosoureas and triazenes, Uracil mustard, Chlormethine, Cyclophosphamide (Cytoxanmi), Ifosfamide, Melphalan, Chlorambucil, Pipobroman, Triethylene-melamine, Triethylenethiophosphoramine, Busulfan, Carmustine. Lomustine, Streptozocin, Dacarbazine, and Temozolomide), antimetabolites (including folic acid antagonists, pyrimidine analogs, purine analogs and adenosine deaminase inhibitors, Methotrexate, 5-Fluorouracil, Floxuridine, Cytarabine, 6-Mercaptopurine, 6-Thioguanine, Fludarabine phosphate, Pentostatine, and Gemcitabine), natural products and their derivatives (including vinca alkaloids, antitumor antibiotics, enzymes, lymphokines and epipodophyllotoxins, Vinblastine, Vincristine, Vindesine, Bleomycin, Dactinomycin, Daunorubicin, Doxorubicin, Epirubicin, Idarubicin, paclitaxel (paclitaxel is commercially available as TAXOL®), Mithramycin, Deoxycoformycin, Mitomycin-C, L-Asparaginase, Interferons (especially IFN-alpha), Etoposide, and Teniposide), hormones and steroids (including synthetic analogs, 17-alpha-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, and Zoladex), and synthetics (including inorganic complexes such as platinum coordination complexes, Cisplatin, Carboplatin, Hydroxyurea, Amsacrine, Procarbazine, Mitotane, Mitoxantrone, Levamisole, and Hexamethylmelamine).

The VAP-1 inhibitor can be used to reduce or delay the recurrence of the condition being treated. In addition, the VAP-1 inhibitor can synergistically enhance the efficacy of the additional treatment, and/or the additional treatment may enhance the efficacy of the VAP-1 inhibitor.

a. VAP-1 Inhibition in Combination with VEGF Modulation

VEGF is a known contributor to angiogenesis and to lymphangiogenesis, increasing the number of capillaries in a given network. Capillary endothelial cells have been shown to proliferate and initiate new vessel tube structures upon stimulation by VEGF. Previous studies have demonstrated that plated endothelial cells presented with VEGF will proliferate, migrate, and form tube structures resembling capillaries.

VEGF has been shown to cause a massive signaling cascade in endothelial cells. Binding to VEGF receptor-2 (VEGFR-2) starts a tyrosine kinase signaling cascade that stimulates the production of factors that variously stimulate vessel permeability (eNOS, producing NO), proliferation/survival (bFGF), migration (ICAMs/VCAMs/MMPs) and finally differentiation into mature blood vessels. Moreover, as noted above, certain cancer cells stop producing an anti-VEGF enzyme, PKG, which shifts the equilibrium of blood vessel growth toward angiogenesis.

Accordingly, the treatment of a VAP-1 inhibitor to inhibit angiogenesis can be combined with an anti-VEGF factor, for example, an anti-VEGF antibody or antibody fragment, nucleic acid, or small molecule. One example of an anti-VEGF factor is the anti-VEGF antibody AVASTIN®. See the URL address: gene.com/gene/products/information/oncology/avastin/index.jsp (available from Genentech, Inc., San Francisco, Calif.). Another example of an anti-VEGF factor is the aptamer MACUGEN® (see the URL address eyetk.com/science/science_vegf.asp), available from Eyetech Pharmaceuticals, Inc., NY, N.Y. Alternatively, the VAP-1 inhibitor may be combined with a VEGF specific RNAi. See the URL address: alnylam.com/therapeutic-programs/programs.asp (available from Alnylam Pharmaceuticals, Cambridge, Mass.). Similarly, the VAP-1 inhibitor may be combined with a small molecule VEGF inhibitor for the treatment of cancer, corneal neovascularization, and/or CNV.

The treatment of a VAP-1 inhibitor to inhibit lymphangiogenesis also can be combined with an anti-VEGF factor, for example, any anti-VEGF factor described above.

b. VAP-1 Inhibition in Combination with PDT

In one aspect, the invention provides an improved PDT-based method for treating angiogenic conditions, such as unwanted CNV and/or lymphatic conditions. An increase in efficacy and/or selectivity of the PDT, and/or reduction or delay of recurrence of the angiogenic condition, such as CNV and/or lymphatic conditions, may be achieved by administering a VAP-1 inhibitor to a subject prior to, concurrent with, or after administration of the photosensitizer. PDT involves administration of a photosensitizer to a mammal in need of such treatment in an amount sufficient to permit an effective amount (i.e., an amount sufficient to facilitate PDT) of the photosensitizer to localize in the target (e.g. the CNV). After administration of the photosensitizer, the target (e.g. the CNV) then is irradiated with laser light under conditions such that the light is absorbed by the photosensitizer. The photosensitizer, when activated by the light, generates singlet oxygen and free radicals, for example, reactive oxygen species, that result in damage to surrounding tissue. For example, PDT-induced damage of endothelial cells results in platelet adhesion and degranulation, leading to stasis and aggregation of blood cells and vascular occlusion. Although this section highlights CNV, it should be understood that PDT applies to other angiogenic conditions. Moreover, this discussion also should be understood to apply to treatment of a lymphangiogenic condition.

A variety of photosensitizers that are useful in PDT include, for example, amino acid derivatives, azo dyes, xanthene derivatives, chlorins, tetrapyrrole derivatives, phthalocyanines, and assorted other photosensitizers. Amino acid derivatives include, for example, 5-aminolevulinic acid (Berg et al. (1997) Photochem. Photobiol. 65: 403-409; El-Far et al. (1985) Cell. Biochem. Function 3, 115-119). Azo dyes, include, for example, Sudan I, Sudan II, Sudan III. Sudan IV, Sudan Black, Disperse Orange, Disperse Red, Oil Red O, Trypan Blue, Congo Red, β-carotene (Mosky et al. (1984) Exp. Res. 155, 389-396). Xanthene derivatives, include, for example, rose bengal.

Chlorins include, for example, lysyl chlorin p6 (Berg et al. (1997) supra) and etiobenzochlorin (Berg et al. (1997) supra), 5, 10, 15, 20-tetra (m-hydroxyphenyl) chlorin (M-THPC), N-aspartyl chlorin e6 (Dougherty et al. (1998) J. Natl. Cancer Inst. 90: 889-905), and bacteriochlorin (Korbelik et al. (1992) J. Photochem. Photobiol. 12: 107-119).

Tetrapyrrole derivatives include, for example, lutetium texaphrin (Lu-Tex, PCI-0123) (Dougherty et al. (1998) supra, Young et al. (1996) Photochem. Photobiol. 63: 892-897), benzoporphyrin derivative (BPD) (U.S. Pat. Nos. 5,171,749, 5,214,036, 5,283,255, and 5,798,349, Jori et al. (1990) Lasers Med. Sci. 5, 115-120), benzoporphyrin derivative mono acid (BPD-MA) (U.S. Pat. Nos. 5,171,749, 5,214,036, 5,283,255, and 5,798,349, Berg et al. (1997) supra, Dougherty et al. (1998) supra), hematoporphyrin (Hp) (Jori et al. (1990) supra), hematoporphyrin derivatives (HpD) (Berg et al. (1997) supra, West et al. (1990) In. J. Radiat. Biol. 58: 145-156), porfimer sodium or Photofrin (PHP) (Berg et al. (1997) supra), Photofrin II (PII) (He et al. (1994) Photochem. Photobiol. 59: 468-473), protoporphyrin IX (PpIX) (Dougherty et al. (1998) supra, He et al. (1994) supra), meso-tetra (4-carboxyphenyl) porphine (TCPP) (Musser et al. (1982) Res. Commun. Chem. Pathol. Pharmacol. 2, 251-259), meso-tetra (4-sulfonatophenyl) porphine (TSPP) (Musser et al. (1982) supra), uroporphyrin I (UROP-I) (El-Far et al. (1985) Cell. Biochem. Function 3, 115-119), uroporphyrin III (UROP-III) (El-Far et al. (1985) supra), tin ethyl etiopurpurin (SnET2), (Dougherty et al. (1998) supra 90: 889-905) and 13,17-bis[1-carboxypropionyl] carbamoylethyl-8-etheny-2-hydroxy-3-hydroxyiminoethylidene-2,7,12,18-tetranethyl 6 porphyrin sodium (ATX-S10(Na)) Mori et al. (2000) JPN. J. CANCER RES. 91:753-759, Obana et al. (2000) Arch. Ophthalmol. 118:650-658, Obana et al. (1999) Lasers Surg. Med. 24:209-222).

Phthalocyanines include, for example, chloroaluminum phthalocyanine (AlPcCl) (Rerko et al. (1992) Photochem. Photobiol. 55, 75-80), aluminum phthalocyanine with 2-4 sulfonate groups (AlPcS2-4) (Berg et al. (1997) supra, Glassberg et al. (1991) Lasers Surg. Med. 11, 432-439), chloro-aluminum sulfonated phthalocyanine (CASPc) (Roberts et al. (1991) J. Natl. Cancer Inst. 83, 18-32), phthalocyanine (PC) (Jori et al. (1990) supra), silicon phthalocyanine (Pc4) (He et al. (1998) Photochem. Photobiol. 67: 720-728, Jori et al. (1990) supra), magnesium phthalocyanine (Mg2+-PC) (Jori et al. (1990) supra), and zinc phthalocyanine (ZnPC) (Berg et al. (1997) supra). Other photosensitizers include, for example, thionin, toluidine blue, neutral red and azure c.

Useful photosensitizers also include, for example, Lutetium Texaphyrin (Lu-Tex), a new generation photosensitizer having favorable clinical properties including absorption at about 730 nm permitting deep tissue penetration and rapid clearance. Lu-Tex is available from Alcon Laboratories, Fort Worth, Tex. Other useful photosensitizers include benzoporhyrin and benzoporphyrin derivatives, for example, BPD-MA and BPD-DA, available from QLT Inc., Vancouver, Canada.

The photosensitizer preferably is formulated into a delivery system that delivers high concentrations of the photosensitizer to the CNV. Such formulations may include, for example, the combination of a photosensitizer with a carrier that delivers higher concentrations of the photosensitizer to CNV and/or coupling the photosensitizer to a specific binding ligand that binds preferentially to a specific cell surface component of the CNV.

The photosensitizer can be combined with a lipid based carrier. For example, liposomal formulations have been found to be particularly effective at delivering the photosensitizer, green porphyrin, and more particularly BPD-MA to the low-density lipoprotein component of plasma, which in turn acts as a carrier to deliver the photosensitizer more effectively to the CNV. Increased numbers of LDL receptors have been shown to be associated with CNV, and by increasing the partitioning of the photosensitizer into the lipoprotein phase of the blood, it may be delivered more efficiently to the CNV. Certain photosensitizers, for example, green porphyrins, and in particular BPD-MA, interact strongly with lipoproteins. LDL itself can be used as a carrier, but LDL is more expensive and less practical than a liposomal formulation. LDL, or preferably liposomes, are thus preferred carriers for the green porphyrins since green porphyrins strongly interact with lipoproteins and are easily packaged in liposomes. Compositions of green porphyrins formulated as lipocomplexes, including liposomes, are described, for example, in U.S. Pat. Nos. 5,214,036, 5,707,608 and 5,798,349. Liposomal formulations of green porphyrin can be obtained from QLT Inc., Vancouver, Canada. It is contemplated that certain other photosensitizers may likewise be formulated with lipid carriers, for example, liposomes or LDL, to deliver the photosensitizer to CNV.

Furthermore, the photosensitizer can be coupled or conjugated to a targeting molecule that targets the photosensitizer to CNV. For example, the photosensitizer may be coupled or conjugated to a specific binding ligand that binds preferentially to a cell surface component of the CNV, for example, neovascular endothelial homing motif. It appears that a variety of cell surface ligands are expressed at higher levels in new blood vessels relative to other cells or tissues.

Endothelial cells in new blood vessels express several proteins that are absent or barely detectable in established blood vessels (Folkman (1995) Nature Medicine 1:27-31), and include integrins (Brooks et al. (1994) Science 264: 569-571; Friedlander et al. (1995) Science 270: 1500-1502) and receptors for certain angiogenic factors like VEGF. In vivo selection of phage peptide libraries have also identified peptides expressed by the vasculature that are organ-specific, implying that many tissues have vascular “addresses” (Pasqualini et al. (1996) Nature 380: 364-366). It is contemplated that a suitable targeting moiety can direct a photosensitizer to the CNV endothelium thereby increasing the efficacy and lowering the toxicity of PDT.

Several targeting molecules may be used to target photosensitizers to new vessel endothelium. For example, α-v integrins, in particular α-v β3 and α-v β5, appear to be expressed in ocular neovascular tissue, in both clinical specimens and experimental models (Corjay et al. (1997) Invest. Ophthalmol. Vis. Sci. 38, S965; Friedlander et al. (1995) supra). Accordingly, molecules that preferentially bind α-v integrins can be used to target the photosensitizer to CNV. For example, cyclic peptide antagonists of these integrins have been used to inhibit neovascularization in experimental models (Friedlander et al. (1996) Proc. Natl. Acad. Sci. USA 93:9764-9769). A peptide motif having an amino acid sequence, in an N- to C-terminal direction, ACDCRGDIXFC (SEQ ID NO: 1)—also know as RGD-4C—has been identified that selectively binds to human α-v integrins and accumulates in tumor neovasculature more effectively than other angiogenesis targeting peptides (Arap et al. (1998) Nature 279:377-380; Ellerby et al. (1999) Nature Medicine 5: 1032-1038). Angiostatin may also be used as a targeting molecule for the photosensitizer. Studies have shown, for example, that angiostatin binds specifically to ATP synthase disposed on the surface of human endothelial cells (Moser et al. (1999) Proc. Natl. Acad. Sci. USA 96:2811-2816).

Clinical and experimental evidence strongly supports a role for vascular endothelial growth factor (VEGF) in ocular new vessel growth, particularly ischemia-associated neovascularization (Adamis et al. (1996) Arch. Ophthalmol. 114:66-71; Tolentino et al. (1996) Arch. Ophthalmol. 114:964-970; Tolentino et al. (1996) Ophthalmology 103:1820-1828). Potential targeting molecules include antibodies that bind specifically to either VEGF or the VEGF receptor (VEGF-2R). Antibodies to the VEGF receptor (VEGFR-2 also known as KDR) may also bind preferentially to neovascular endothelium. VEGF receptor 3 is known to be present on lymph vessels, so a PDT method directed to lymph vessels could employ antibodies to VEGF receptor 3.

The targeting molecule may be synthesized using methodologies known and used in the art. For example, proteins and peptides may be synthesized using conventional synthetic peptide chemistries or expressed as recombinant proteins or peptides in a recombinant expression system (see, for example, “Molecular Cloning” Sambrook et al. eds, Cold Spring Harbor Laboratories). Similarly, antibodies may be prepared and purified using conventional methodologies, for example, as described in “Practical Immunology”, Butt, W. R. ed., 1984 Marcel Deckker, New York and “Antibodies, A Laboratory Approach” Harlow et al., eds. (1988), Cold Spring Harbor Press. Once created, the targeting agent may be coupled or conjugated to the photosensitizer using standard coupling chemistries, using, for example, conventional cross linking reagents, for example, heterobifunctional cross linking reagents available, for example, from Pierce, Rockford, Ill.

Once formulated, the photosensitizer may be administered in any of a wide variety of ways, for example, orally, parenterally, or rectally. Parenteral administration, such as intravenous, intralymphatic, intramuscular, or subcutaneous, is preferred. Intravenous injection is especially preferred. The dose of photosensitizer can vary widely depending on the tissue to be treated; the physical delivery system in which it is carried, such as in the form of liposomes; or whether it is coupled to a target-specific ligand, such as an antibody or an immunologically active fragment.

It should be noted that the various parameters used for effective, selective photodynamic therapy in the invention are interrelated. Therefore, the dose should also be adjusted with respect to other parameters, for example, fluence, irradiance, duration of the light used in PDT, and time interval between administration of the dose and the therapeutic irradiation. All of these parameters should be adjusted to produce significant damage to CNV without significant damage to the surrounding tissue.

Typically, the dose of photosensitizer used is within the range of from about 0.1 to about 20 mg/kg, preferably from about 0.15 to about 5.0 mg/kg, and even more preferably from about 0.25 to about 2.0 mg/kg. Furthermore, as the dosage of photosensitizer is reduced, for example, from about 2 to about 1 mg/kg in the case of green porphyrin or BPD-MA, the fluence required to close CNV may increase, for example, from about 50 to about 100 Joules/cm². Similar trends may be observed with the other photosensitizers discussed herein.

After the photosensitizer has been administered, the CNV is irradiated at a wavelength typically around the maximum absorbance of the photosensitizer, usually in the range from about 550 nm to about 750 nm. A wavelength in this range is especially preferred for enhanced penetration into bodily tissues. Preferred wavelengths used for certain photosensitizers include, for example, about 690 nm for benzoporphyrin derivative mono acid, about 630 nm for hematoporphyrin derivative, about 675 nm for chloro-aluminum sulfonated phthalocyanine, about 660 nm for tin ethyl etiopurpurin, about 730 nm for lutetium texaphyrin, about 670 nm for ATX-S10(NA), about 665 nm for N-aspartyl chlorin e6, and about 650 nm for 5, 10, 15, 20-tetra (m-hydroxyphenyl) chlorin.

As a result of being irradiated, the photosensitizer in its triplet state is thought to interact with oxygen and other compounds to form reactive intermediates, such as singlet oxygen and reactive oxygen species, which can disrupt cellular structures. Possible cellular targets include the cell membrane, mitochondria, lysosomal membranes, and the nucleus. Evidence from tumor and neovascular models indicates that occlusion of the vasculature is a major mechanism of photodynamic therapy, which occurs by damage to the endothelial cells, with subsequent platelet adhesion, degranulation, and thrombus formation.

The fluence during the irradiating treatment can vary widely, depending on the type of photosensitizer used, the type of tissue, the depth of target tissue, and the amount of overlying fluid or blood. Fluences preferably vary from about 10 to about 400 Joules/cm² and more preferably vary from about 50 to about 200 Joules/cm². The irradiance varies typically from about 50 mW/cm² to about 1800 mW/cm², more preferably from about 100 mW/cm² to about 900 mW/cm², and most preferably in the range from about 150 mW/cm² to about 600 mW/cm². It is contemplated that for many practical applications, the irradiance will be within the range of about 300 mW/cm² to about 900 mW/cm². However, the use of higher irradiances may be selected as effective and having the advantage of shortening treatment times.

The time of light irradiation after administration of the photosensitizer may be important as one way of maximizing the selectivity of the treatment, thus minimizing damage to structures other than the target tissues. The optimum time following photosensitizer administration until light treatment can vary widely depending on the mode of administration, the form of administration such as in the form of liposomes or as a complex with LDL, and the type of target tissue. For example, bcnzoporphyrin derivative typically becomes present within the target neovasculature within one minute post administration and persists for about fifty minutes, lutetium texaphyrin typically becomes present within the target neovasculature within one minute post administration and persists for about twenty minutes, N-aspartyl chlorin e6 typically becomes present within the target neovasculature within one minute post administration and persists for about twenty minutes, and rose bengal typically becomes present in the target vasculature within one minute post administration and persists for about ten minutes.

Effective vascular closure generally occurs at times in the range of about one minute to about three hours following administration of the photosensitizer. However, as with green porphyrins, it is undesirable to perform the PDT within the first five minutes following administration to prevent undue damage to retinal vessels still containing relatively high concentrations of photosensitizer.

The efficacy of PDT may be monitored using conventional methodologies, for example, via fundus photography or angiography. Closure can usually be observed angiographically by hypofluorescence in the treated areas in the early angiographic frames. During the later angiographic frames, a corona of hyperfluorescence may begin to appear which then fills the treated area, possibly representing leakage from the adjacent choriocapillaris through damaged retinal pigment epithelium in the treated area. Large retinal vessels in the treated area typically perfuse following photodynamic therapy. Minimal retinal damage is generally found on histopathologic correlation and is dependent on the fluence and the time interval after irradiation that the photosensitizer is administered. It is contemplated that the choice of appropriate photosensitizer, dosage, mode of administration, formulation, timing post administration prior to irradiation, and irradiation parameters may be determined empirically.

The administration of a VAP-1 inhibitor may be used before, during, and/or after PDT treatment to enhance the success of inhibiting angiogenic conditions, such as CNV, and/or lymphatic conditions.

c. VAP-1 Inhibition in Combination with an Apoptosis Factor

The efficacy of VAP-1 inhibition of angiogenesis, alone or in combination with another therapy, for example PDT, may be enhanced by combination with administration of an apoptosis-modulating factor. Similarly, the efficacy of VAP-1 inhibition of lymphangiogenesis, alone or in combination with another therapy, may be enhanced by combination with administration of an apoptosis-modulating factor. An apoptosis-modulating factor can be any factor, for example, a protein (for example a growth factor or antibody), peptide, nucleic acid (for example, an antisense oligonucleotide or siRNA), peptidyl nucleic acid (for example, an antisense molecule), organic molecule or inorganic molecule, that induces or represses apoptosis in a particular cell type. For example, it may be advantageous to prime the apoptotic machinery of endothelial cells (e.g. CNV endothelial cells) with an inducer of apoptosis prior to treatment so as to increase their sensitivity to treatment. Endothelial cells primed in this manner are contemplated to be more susceptible to treatments such as PDT. This approach may also reduce the light dose (fluence) required to achieve CNV closure in PDT and thereby decrease the level of damage on surrounding cells such as RPE. Alternatively, the cells outside the CNV may be primed with a repressor of apoptosis so as to decrease their sensitivity to the treatment. Although this section highlights CNV, it should be understood that apoptosis modulators can be used in combination with VAP-1 inhibitors to treat other angiogenic conditions and/or lymphangiogenic conditions.

Apoptosis involves the activation of a genetically determined cell suicide program that results in a morphologically distinct form of cell death characterized by cell shrinkage, nuclear condensation, DNA fragmentation, membrane reorganization and blebbing (Kerr et al. (1972) Br. J. Cancer 26: 239-257). At the core of this process lies a conserved set of proenzymes, called caspases, and two important members of this family are caspases 3 and 7 (Nicholson et al. (1997) TIBS 22:299-306). Monitoring their activity can be used to assess on-going apoptosis.

It has been suggested that apoptosis is associated with the generation of reactive oxygen species, and that the product of the Bcl-2 gene protects cells against apoptosis by inhibiting the generation or the action of the reactive oxygen species (Hockenbery et al. (1993) Cell 75: 241-251, Kane et al. (1993) Science 262: 1274-1277, Veis et al. (1993) Cell 75: 229-240, Virgili et al. (1998) Free Radicals Biol. Med. 24: 93-101). Bcl-2 belongs to a growing family of apoptosis regulatory gene products, which may either be death antagonists (Bcl-2, Bcl-xL) or death agonists (Bax, Bak) (Kroemer et al. (1997) Nat. Med. 3: 614-620). Control of cell death appears to be regulated by these interactions and by constitutive activities of the various family members (Hockenbery et al. (1993) Cell 75: 241-251). Several apoptotic pathways may coexist in mammalian cells that are preferentially activated in a stimulus-, stage-, context-specific and cell-type manner (Hakem et al. (1998) Cell 94: 339-352).

The apoptosis-inducing factor preferably is a protein or peptide capable of inducing apoptosis in cells, for example, endothelial cells, disposed in the CNV. One apoptosis inducing peptide comprises an amino sequence having, in an N- to C-terminal direction, KLAKLAKKLAKLAK (SEQ ID NO: 2). This peptide reportedly is non-toxic outside cells, but becomes toxic when internalized into targeted cells by disrupting mitochondrial membranes (Ellerby et al. (1999) supra). This sequence may be coupled, either by means of a cross-linking agent or a peptide bond, to a targeting domain, for example, the amino acid sequence known as RGD-4C (Ellerby et al. (1999) supra) that reportedly can direct the apoptosis-inducing peptide to endothelial cells. Other apoptosis-inducing factors include, for example, constatin (Kamphaus et al. (2000) J. Biol. Chem. 14: 1209-1215), tissue necrosis factor α (Lucas et al. (1998) Blood 92: 4730-4741) including bioactive fragments and analogs thereof, cycloheximide (O'Connor et al. (2000) Am. J. Pathol. 156: 393-398), tunicamycin (Martinez et al. (2000) Adv. Exp. Med. Biol. 476: 197-208), and adenosine (Harrington et al. (2000) Am. J. Physiol. Lung Cell Mol. Physiol. 279: 733-742). Furthermore, other apoptosis-inducing factors may include, for example, anti-sense nucleic acid or peptidyl nucleic acid sequences that reduce or turn off the expression of one or more of the death antagonists, for example (Bcl-2, Bcl-xL). Antisense nucleotides directed against Bcl-2 have been shown to reduce the expression of Bcl-2 protein in certain lines together with increased phototoxicity and susceptibility to apoptosis during PDT (Zhang et al. (1999) Photochem. Photobiol. 69: 582-586). Furthermore, an 18mer phosphorothiate oligonucleotide complementary to the first six codons of the Bcl-2 open reading frame, and known as G3139, is being tested in humans as a treatment for non-Hodgkins' lymphoma.

Apoptosis-repressing factors include, survivin, including bioactive fragments and analogs thereof (Papapetropoulos et al. (2000) J. Biol. Chem. 275: 9102-9105), CD39 (Goepfert et al. (2000) Mol. Med. 6: 591-603), BDNF (Caffe et al. (2001) Invest. Ophthalmol. Vis. Sci. 42: 275-82), FGF2 (Bryckaert et al. (1999) Oncogene 18: 7584-7593), Caspase inhibitors (Ekert et al. (1999) Cell Death Differ 6: 1081-1068) and pigment epithelium-derived growth factor including bioactive fragments and analogs thereof. Furthermore, other apoptosis-repressing factors may include, for example, anti-sense nucleic acid or peptidyl nucleic acid sequences that reduce or turn off the expression of one or more of the death agonists, for example (Bax, Bak).

To the extent that the apoptosis-modulating factor is a protein or peptide, nucleic acid, peptidyl nucleic acid, or organic or inorganic compound, it may be synthesized and purified by one or more the methodologies described relating to the synthesis of the VAP-1 inhibitor above.

The type and amount of apoptosis-modulating factor to be administered may depend upon the treatment and cell type to be treated. It is contemplated, however, that optimal apoptosis-modulating factors, modes of administration and dosages may be determined empirically. The apoptosis modulating factor may be administered in a pharmaceutically acceptable carrier or vehicle so that administration does not otherwise adversely affect the recipient's electrolyte and/or volume balance. The carrier may comprise, for example, physiologic saline.

Protein, peptide or nucleic acid based apoptosis modulators can be administered at doses ranging, for example, from about 0.001 to about 500 mg/kg, more preferably from about 0.01 to about 250 mg/kg, and most preferably from about 0.1 to about 100 mg/kg. For example, nucleic acid-based apoptosis inducers, for example, G318, may be administered at doses ranging from about 1 to about 20 mg/kg daily. Furthermore, antibodies may be administered intravenously at doses ranging from about 0.1 to about 5 mg/kg once every two to four weeks. With regard to intravitreal administration, the apoptosis modulators, for example, antibodies, may be administered periodically as bolus dosages ranging from about 10 μg to about 5 mg/eye and more preferably from about 100 μg to about 2 mg/eye.

The apoptosis-modulating factor can be administered before, during or after VAP-1 inhibitor administration. To the extent the apoptosis-modulating factor is used with PDT, it preferably is administered to the mammal prior to PDT (although it may be administered during or after PDT). Accordingly, it is preferable to administer the apoptosis-modulating factor prior to administration of the photosensitizer. The apoptosis-modulating factor, like the photosensitizer and VAP-1 inhibitor, may be administered in any one of a wide variety of ways, for example, orally, parenterally, or rectally. However, parenteral administration, such as intravenous, intramuscular, subcutaneous, and intravitreal is preferred. Administration may be provided as a periodic bolus (for example, intravenously or intravitreally) or by continuous infusion from an internal reservoir (for example, bioerodable implant disposed at an intra- or extra-ocular location) or an external reservoir (for example, and intravenous bag). The apoptosis modulating factor may be administered locally, for example, by continuous release from a sustained release drug delivery device immobilized to an inner wall of the eye or via targeted trans-scleral controlled release into the choroid (see, PCT/US00/00207).

### IV. VAP-1 Inhibitor Administration and Dosing

The type and amount of VAP-1 inhibitor to be administered will depend upon the particular treatment and cell type to be treated. It is contemplated, however, that optimal VAP-1 inhibitors, modes of administration and dosages may be determined empirically. The VAP-1 inhibitor may be administered in a pharmaceutically acceptable carrier or vehicle so that administration does not otherwise adversely affect the recipient's electrolyte and/or volume balance.

Small molecule VAP-1 inhibitors may be administered at doses ranging, for example, from 1-1500 mg/m², for example, about 3, 30, 60, 90, 180, 300, 600, 900, 1200 or 1500 mg/m². Protein, peptide or nucleic acid based VAP-1 inhibitors can be administered at doses ranging, for example, from about 0.001 to about 500 mg/kg, more preferably from about 0.01 to about 250 mg/kg, and most preferably from about 0.1 to about 100 mg/kg. The VAP-1 inhibitor may be administered in any one of a wide variety of routes, for example, by a topical, transdermal, intraperitoneal, intracranial, intracerebroventricular, intracerebral, intravaginal, intrauterine, oral, rectal, parenteral (e.g., intravenous, intralymphatic, intraspinal, subcutaneous or intramuscular), and intravitreal route. With regard to intravitreal administration, the VAP-1 inhibitor, for example, anti-VAP-1 neutralizing antibody, may be administered periodically as boluses at dosages ranging from about 10 μg to about 5 mg/eye and more preferably from about 100 μg to about 2 mg/eye.

Formulations suitable for administration of a VAP-1 inhibitor may include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. The formulations may also be presented in continuous release vehicles. Extemporaneous injection solutions and suspensions may be prepared from sterile powders, granules and tablets of the kind previously described. The excipient formulations conveniently may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

The VAP-1 inhibitor may be administered in a single bolus, in multiple boluses, or in a continuous release format. Accordingly, formulations may contain a single dose or unit, multiple doses or units, or a dosage for extended delivery of the VAP-1 inhibitor. It should be understood that in addition to the ingredients mentioned above, the formulations of the present invention may include other agents conventional in the art having regard to the type of delivery in question. For example, the carrier may comprise, for example, physiologic saline, or may comprise components necessary for, for example, administration as an ointment, administration via encapsulated microspheres or liposomes, or administration via a device for continuous release.

The VAP-1 inhibitor also may be administered systemically or locally. For example, administration may be provided locally as a single bolus, for example, by parenteral or intravitreal injection or by deposition to a site of interest such as a location in the eye or adjacent to or within a tumor. Administration may be provided systemically as a periodic bolus, for example, intravenously, intralymphatically, or intravitreally, or locally as a periodic bolus, for example, by injection, deposition, or as periodic infusion from an internal reservoir or from an external reservoir (for example, from an intravenous bag). The VAP-1 inhibitor may be administered systemically or locally in a continuous release format, for example, from a bioerodable implant or from a sustained release drug delivery device. For example, in certain embodiments, a delivery device can be used for delivery of the VAP-1 inhibitor into the eye or via targeted trans-scleral controlled release (see, PCT/US00/00207) for treatment of the eye. In certain embodiments, particularly those directed to treatment of ocular diseases, such as corneal angiogenesis, the VAP-1 inhibitor may be administered from a contact lens. The contact lens may be pre-soaked with the VAP-1 inhibitor prior to use of the contact lens. Alternatively, in certain embodiments, particularly those directed to treatment of tumors, the VAP-1 inhibitor may be incorporated into a biodegradable polymer that may be implanted at the site of a tumor. Alternatively, a biodegradable polymer may be implanted so that the VAP-1 inhibitor is slowly released systemically rather than locally. Such biodegradable polymers and their use are known in the art and described, for example, in detail in Brem et al. (1991) J. Neurosurg. 74:441-446. Osmotic minipumps may also be used to provide controlled delivery of high concentrations of VAP-1 inhibitor through cannulae to the site of interest, such as directly into a metastatic growth or into the vascular or lymphatic supply of a tumor, or to a location in the body that facilitates systemic release.

The present invention, therefore, includes the use of a VAP-1 inhibitor in the preparation of a medicament for treating an a condition associated with angiogenesis, for example, cancer, ocular angiogenesis, corneal neovascularization, and/or CNV. The present invention also includes the use of a VAP-1 inhibitor in the preparation of a medicament for treating an a condition associated with lymphangiogenesis, for example, cancer, ocular lymphangiogenesis, and lymphangiogenesis of the cornea. The VAP-1 inhibitor may be provided in a kit which optionally may comprise a package insert with instructions for how to treat such a condition.

In combination treatments, the VAP-1 inhibitor may be administered to the subject prior to other treatment(s). It may alternatively or additionally be administered during and/or after the other treatment(s). In combination with PDT therapy, the VAP-1 inhibitor may be administered before, during, or after PDT therapy. It may be preferable to administer the VAP-1 inhibitor prior to administration of the photosensitizer. For a combination product with PDT, a composition may provide both a photosensitizer and a VAP-1 inhibitor. The composition may also comprise a pharmaceutically acceptable carrier or excipient. Thus, the present invention includes a pharmaceutically acceptable composition comprising a photosensitizer and a VAP-1 inhibitor; as well as the composition for use in medicine. However, the VAP-1 inhibitor and a photosensitizer may be administered separately. Instructions for such administration may be provided with the VAP-1 inhibitor and/or with the photosensitizer. If desired, the VAP-1 inhibitor and photosensitizer may be provided together in a kit, optionally including a package insert with instructions for use. The VAP-1 inhibitor and photosensitizer preferably are provided in separate containers.

The VAP-1 inhibitor may be used in combination with other compositions and procedures for the treatment of a cancer. For example, a tumor may be treated conventionally with surgery, radiation or chemotherapy combined with the VAP-1 inhibitor. Optionally, the VAP-1 inhibitor may also be subsequently administered to the patient to extend the dormancy of metastases and to stabilize any residual primary tumor. Administration of therapeutics directed to cancer treatment are known in the art. For example, radiation therapy, including x-rays or gamma rays, are delivered from either an externally applied beam or by implantation of tiny radioactive sources. Administration of chemotherapeutic agents are well known and described in standard literature, for example, “Physicians' Desk Reference” (PDR), e.g., 2004 edition (Thomson PDR, Montvale, N.J. 07645-1742, USA). A VAP-1 inhibitor may be administered in combination with any known anti-cancer treatment and may have dosage ranges described herein. Combinations of the instant invention may be used sequentially with known pharmaceutically acceptable agent(s) when a multiple combination formulation is inappropriate.

The foregoing methods and compositions of the invention are useful in treating angiogenesis and thereby ameliorate the symptoms of various disorders associated with angiogenesis including, for example, cancer (e.g. tumor growth or metastasis), corneal neovascularization, unwanted choroidal neovasculature, and AMD. The foregoing methods and compositions of the invention are also useful in treating lymphangiogenesis and thereby ameliorate the symptoms of various disorders associated with lymphangiogenesis including, for example, cancer (e.g. tumor growth or metastasis) and growth of lymph vessels into the cornea. It is contemplated that the same methods and compositions may also be useful in treating other forms of angiogenesis and/or lymphangiogenesis, as described above.

The invention is illustrated further by reference to the following non-limiting examples.

### EXAMPLES

### Example 1. VAP-1 Blockade Suppresses CNV

VAP-1 is an endothelial cell adhesion molecule involved in leukocyte recruitment. Macrophages play an important role in the development of choroidal neovascularization (CNV), an integral component of age-related macular degeneration (AMD). Previously, it was shown that VAP-1 is involved in ocular inflammation. In this Example, the expression of VAP-1 in the choroid and its role in CNV development was investigated.

These data show that VAP-1 was expressed in the choroid, exclusively in the vessels, and colocalized in the vessels of the CNV lesions. In addition, these data show that VAP-1 blockade with a specific inhibitor (Compound II, described above) significantly decreased CNV size, fluorescent angiographic leakage, and the accumulation of macrophages in the CNV lesions. Further, these data show that VAP-1 blockade significantly reduced the expression of inflammation-associated molecules such as tumor necrosis factor (TNF-α), monocyte chemoattractant protein (MCP-1) and intercellular adhesion molecule (ICAM-1). Overall, these data provide evidence for an important role of VAP-1 in the recruitment of macrophages to CNV lesions and identifies VAP-1 inhibition as a therapeutic strategy in the treatment of CNV.

a. Background

Choroidal neovascularization (CNV) is the main cause of severe vision loss in patients with age-related macular degeneration (AMD). There is evidence that inflammatory cells are critically involved in the formation of CNV lesions and play a role in the pathogenesis of age-related macular degeneration. Inflammatory cells have been found in the CNV lesions that were surgically excised from AMD patients and in autopsy eyes with CNV. In particular, macrophages have been implicated in the pathogenesis of AMD due to their spatiotemporal distribution in the proximity of the CNV lesion both in humans and experimental models.

Macrophages are known to be a source of proangiogenic and inflammatory cytokines, such as vascular endothelial growth factor (VEGF) and tumor necrosis factor (TNF)-α, both of which significantly contribute to the pathogenesis of CNV. Most of the macrophages found in the proximity of the laser-induced CNV lesions likely are derived from newly recruited peripheral blood monocytes and not resident macrophages. Since macrophages play such a critical role in CNV formation, prevention of monocyte recruitment and infiltration into ocular tissues may ameliorate the development of CNV.

VAP-1 is an endothelial cell adhesion molecule involved in leukocyte recruitment. In ocular tissues, VAP-1 has been shown to localize on the endothelial cells of the retina and play a critical role in the recruitment of leukocytes under both normal and inflammatory conditions. Recently, it has been reported that VAP-1 antibody treatment suppresses recruitment of monocyte/macrophage lineages in vivo, suggesting an important role for VAP-1 in macrophage transmigration under pathologic conditions.

Therefore, these investigations were carried out to show that VAP-1 regulates macrophage recruitment into ocular tissues and that its blockade attenuates CNV formation. Specifically, these investigations identified the expression and distribution of VAP-1 in the choroidal tissues of normal and laser-injured animals, and investigated the role of VAP-1 in CNV formation using a specific inhibitor identified as Compound II, above.

b. Methods

Experimental Animals

For reverse transcription polymerase chain reaction (RT-PCR) detection and immunofluorescence staining of VAP-1 in the choroid, Lewis rats (8-10 weeks old, Charles River Laboratories, Inc., Wilmington, Mass.) were used. To generate CNV in the laser injury model, Brown-Norway rats (10-12 weeks old, Charles River Laboratories, Inc., Wilmington, Mass.) were used. Rats were housed in plastic cages in a climate controlled animal facility and were fed laboratory chow and water ad libitum. All animal experiments were conducted in accordance with the ARVO Statement for the Use of Animals in Ophthalmic and Vision Research.

RNA Extraction and RT-PCR

Lewis rats were euthanized by overdose anesthesia and perfused with PBS (500 ml/kg body weight (BW)). Eyes were immediately enucleated and the retinal pigment epithelium (RPE)-choroid complex was obtained from the rat eyes and homogenized in extraction reagent (TRIzol Reagent; Invitrogen, Carlsbad, Calif.). As a control, the retinal tissues were separately obtained and processed. Total RNA was prepared according to the manufacturer's protocol, and equal amounts (1 μg) of total RNA were reverse transcribed with a First-Strand cDNA synthesis kit (GE Healthcare, Buckinghamshire, UK) at 37° C. for 1 hour in a 15 μl reaction volume. PCR was performed using Platinum PCR SuperMix (Invitrogen) with a thermal controller (GeneAmp PCR System 9700; Applied Biosystems, Foster city, CA). The thermal cycle was 1 minute at 94° C., 1 minute at 55° C. and 1 minute at 72° C., followed by 5 minutes at 72° C. The reaction was performed for 35 cycles for amplification of VAP-1 and 30 cycles for glyceraldehyde-3-phosphate dehydrogenase (GAPDH) with previously designed primers. The nucleotide sequences of the PCR primers were 5′-GAC CCT CGG ACA ACT GTG TCT T-3′ (forward) (SEQ ID NO: 3) and 5′-GCG TT GTA GAA GCA ACA GTG A-3′ (reverse) (SEQ ID NO: 4) for VAP-1 and 5′-TGG CAC AGT CAA GGC TGA GA-3′ (forward) (SEQ ID NO: 5) and 5′-CUT CTG AGT GGC AGT GAT GG-3′ (reverse) (SEQ ID NO: 6) for glyceraldehyde-3-phosphate dehydrogenase (GAPDH). PCR products were analyzed by electrophoresis in a 1.5% agarose gel and stained with ethidium bromide (0.2 μg/ml). The expected sizes of the amplified cDNA fragments of VAP-1 and GAPDH were 341 bp and 387 bp, respectively. Band densities were quantified using NIH Image 1.41 software (available by ftp from zippy.nimh.nih.gov/or from the web site, rsb.info.nih.gov/nih-image: developed by Wayne Rasband, National Institutes of Health, Bethesda, Md.). The expression level of VAP-1 mRNA was normalized by that of GAPDH.

Induction of CNV

Brown-Norway rats were anesthetized with 0.2-0.3 ml of a 50:50 mixture of 100 mg/ml Ketamine and 20 mg/ml Xylazine. Pupils were dilated with 5.0% Phenylephrine and 0.8% Tropicamide. CNV was induced with a 532 nm laser (Oculight GLx, Iridex, Mountain View, Calif.). Six laser spots (150 mW, 100 μm, 100 msec) were placed in each eye using a slit-lamp delivery system and a cover glass as a contact lens. Production of a bubble at the time of laser confirmed the rupture of the Bruch's membrane.

Immunohistochemistry

Seven days after laser injury paraffin sections of the choroidal-scleral complex and OCT compound-embedded sections of the rat eyes were prepared. The sections were incubated with blocking solution (Invitrogen) and then reacted with either mouse monoclonal antibody against rat VAP-1 (1:200; BD biosciences, Franklin Lakes, N.J.) or rabbit polyclonal antibody against rat VAP-1 (1:200; Santa Cruz Biotechnology, Inc). For the OCT-embedded sections, biotinylated-isolectin B4 (1:100; Sigma, St. Louis, Mo.) was also used to visualize the structure of the vessels in the CNV lesions. Thereafter, the sections were incubated for 30 min. at room temperature with secondary antibodies (ALEXA FLUOR® 546, Molecular Probes, Eugene, Oreg.) or FITC-conjugated streptavidin (Jackson ImmunoResearch Laboratories, Inc., West Grove, Pa.), and mounted with Vectashield mounting media with 4′,6-diamino-2-phenylindole (DAPI) (Vector Laboratories, Burlingame, Calif.). Photomicrographs were taken with a digital high sensitivity camera (Hamamatsu, ORCA-ER C4742-95, Japan) thorough an upright fluorescent microscope (DM RXA; Leica, Solms, Germany). As a negative control, the primary antibodies were replaced with non-immune mouse IgG (Dako North America, Inc., Carpinteria, Calif.).

VAP-1 Inhibition

To block VAP-1, a specific VAP-1 inhibitor, Compound 11 described above, was used (R-tech Ueno, Ltd., Tokyo, Japan). After laser injury, the inhibitor (0.3 mg/kg BW) was administered to the animals by daily i.p. injections. As a control, some animals received the same regimen for the vehicle solution alone. Compound II has an IC₅₀ of 0.007 μM against human and 0.008 μM against rat semicarbazide-sensitive amine oxidase (SSAO), whereas its IC₅₀ against the functionally related monoamine oxidase (MAO)-A and MAO-B is greater than 10 μM.

Fluorescein Angiography

Seven days after laser injury, vascular leakage from the CNV lesions was assessed using fluorescein angiography (FA), as described previously (Zambarakji et al. (2001) IOVS 42: 1553-60). Briefly, FA was performed in anesthetized animals from VAP-1 inhibitor- or vehicle-treated groups, using a digital fundus camera (Model TRC 50 IA; Topcon, Paramus, N.J.). Fluorescein injections were performed intraperitoneally (0.2 ml of 2% fluorescein; Akorn, Decatur, Ill.).

FA images were evaluated by two masked retina specialists, as previously described by Zambarakji et al. Briefly, the grading criteria were: Grade-0 lesions had no hyperfluorescence; Grade-I lesions exhibited hyperfluorescence without leakage; Grade-IIA lesions exhibited hyperfluorescence in the early or midtransit images and late leakage; and Grade-IIB lesions showed bright hyperfluorescence in the transit images and late leakage beyond the treated areas. The Grade-IIB lesions were defined as clinically significant, as described previously.

Choroidal Flatmount Preparation

One week or two weeks after laser injury and treatment with VAP-1 inhibitor or vehicle, the size of CNV lesions was quantified using choroidal flat mounts. Briefly, rats were anesthetized and perfused through the left ventricle with 20 ml PBS followed by 20 ml of 5 mg/ml fluorescein labeled dextran (FITC-dextran; MW=2×106, SIGMA) in 1% gelatin. The eyes were enucleated and fixed in 4% paraformaldehyde for 3 hours. The anterior segment and retina were removed from the eyecup. Four to six relaxing radial incisions were made, and the remaining RPE-choroidal-scleral complex was flatmounted with Vectashield Mounting Medium (Vector Laboratories) and coverslipped. Pictures of the choroidal flat mounts were taken and Openlab software (Improvision, Boston, Mass.) was used to measure the magnitude of the hyperfluorescent areas corresponding to the CNV lesions. The average size of the CNV lesions was then determined and used for the evaluation.

Quantification of the Macrophage Infiltration

At 1, 3, and 7 days after laser injury and treatment with either VAP-1 inhibitor or vehicle solution, animals were perfused with 200 ml of PBS/kg BW under deep anesthesia. Subsequently, eyes were enucleated and fixed overnight with 4% PFA, and 10 μm frozen sections of the posterior segment, including the center portion of CNV lesions (6 lesions per eye), were prepared and pre-blocked (PBS containing 10% goat serum, 0.5% gelatin, 3% BSA, and 0.2% Tween 20). The sections were incubated with mouse monoclonal antibody for ED-1, rat homologue of human CD68 (1:100; BD Pharmingen, San Diego, Calif.), and subsequently incubated with the secondary antibody (goat antimouse IgG conjugated to ALEXA FLUOR® 488, Molecular Probes). Sections were mounted with Vectashield mounting media (Vector Laboratories). The photographs of CNV lesions were taken, and the numbers of ED-1-positive cells were counted. To obtain a quantitative index of macrophage numbers in CNV lesions, an optical density plot of the selected area was generated by a histogram graphing tool in the Photoshop imageanalysis software (version 6.0; Adobe Systems, Mountain View, Calif.), as described in the literature (for example, Sakurai et al. (2003) IOVS 44: 3578-85). Image analysis was performed in a masked fashion.

Enzyme-Linked Immunosorbent Assay for TNF-α, MCP-1 and ICAM-1

The RPE-choroid complex was carefully isolated from eyes 3 days after photocoagulation and placed in 300 μl of lysis buffer supplemented with protease inhibitors and sonicated. The lysate was centrifuged at 15,000 rpm for 15 minutes at 4° C. and the levels of TNF-α, monocyte chemotactic protein (MCP)-1, and intercellular adhesion molecule (ICAM)-1 were determined with rat TNF-α (BD bioscience), MCP-1 (BD bioscience) and ICAM-1 (R&D Systems, Minneapolis, Minn.) enzyme-linked immunosorbent assay (ELISA) kits according to the manufacturers' protocols. Total protein concentration was determined using a Bio-Rad Protein Assay Kit (Bio-Rad Laboratories Hercules, Calif.) and dilutions of bovine serum albumin (Bio-Rad Laboratories) as standards.

Statistical Analysis

All results are expressed as mean±SEM with n-numbers as indicated. Student's t-test was used for statistical comparison between the groups. The results of the FA gradings were compared using the chi-square test. Differences between the means were considered statistically significant when the probability values were <0.05.

c. Results

VAP-1 Expression in the Choroid and CNV

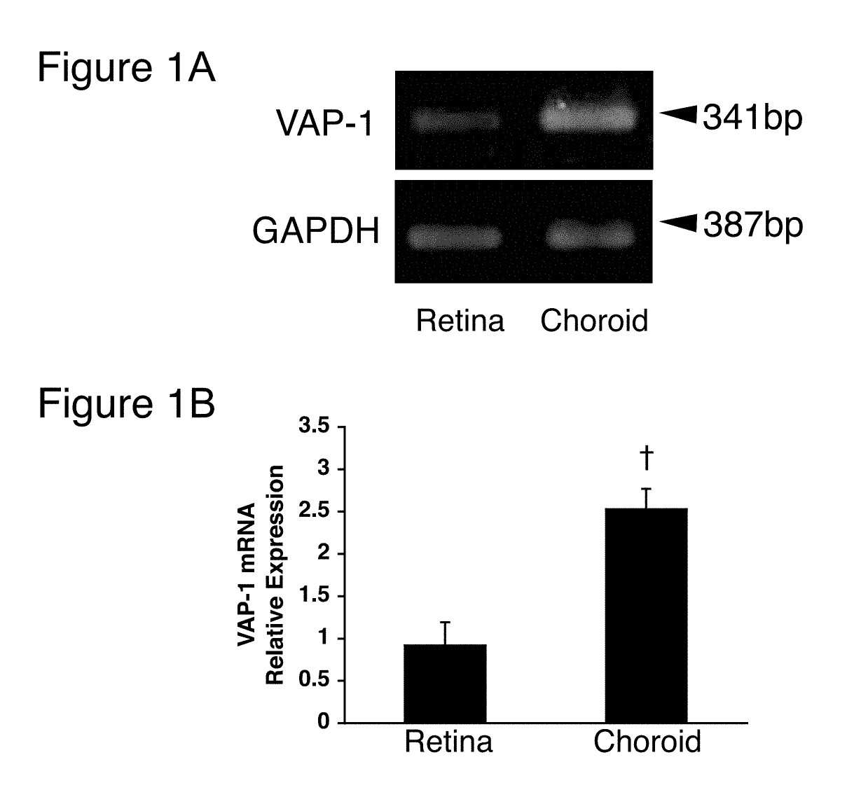

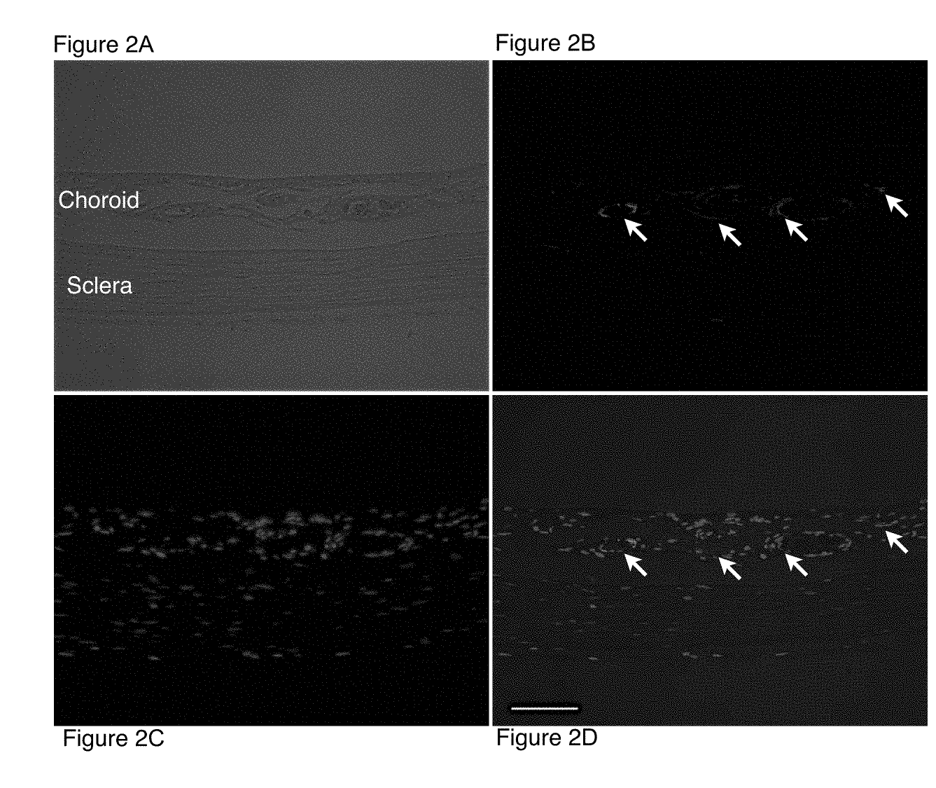

To determine whether VAP-1 is expressed in the choroid, the level of its mRNA expression was examined by RT-PCR and its protein expression was examined by immunofluorescence staining. Since choroidal tissues and RPE cells usually contain melanin, which binds to thermostable DNA polymerase and interferes with the PCR amplification, albino rats that lack melanin were used. In line with a previous study, VAP-1 mRNA was detectable in the retina under normal conditions (FIG. 1A). However, RT-PCR revealed constitutive VAP-1 mRNA expression in the RPE-choroid complex under normal conditions (FIG. 1A). Semi-quantitative analysis of the band intensity showed a 2.8-fold higher expression of VAP-1 mRNA in the RPE-choroid complex compared to that in the retinal tissues (n=4 in each group, p<0.01, FIG. 1B). In addition, immunofluorescence staining of sections from the eyes of normal animals showed the expression of VAP-1 protein in the choroid and that VAP-1 was exclusively localized in the vessels (FIGS. 2A-2D).

Role of VAP-1 in CNV Formation

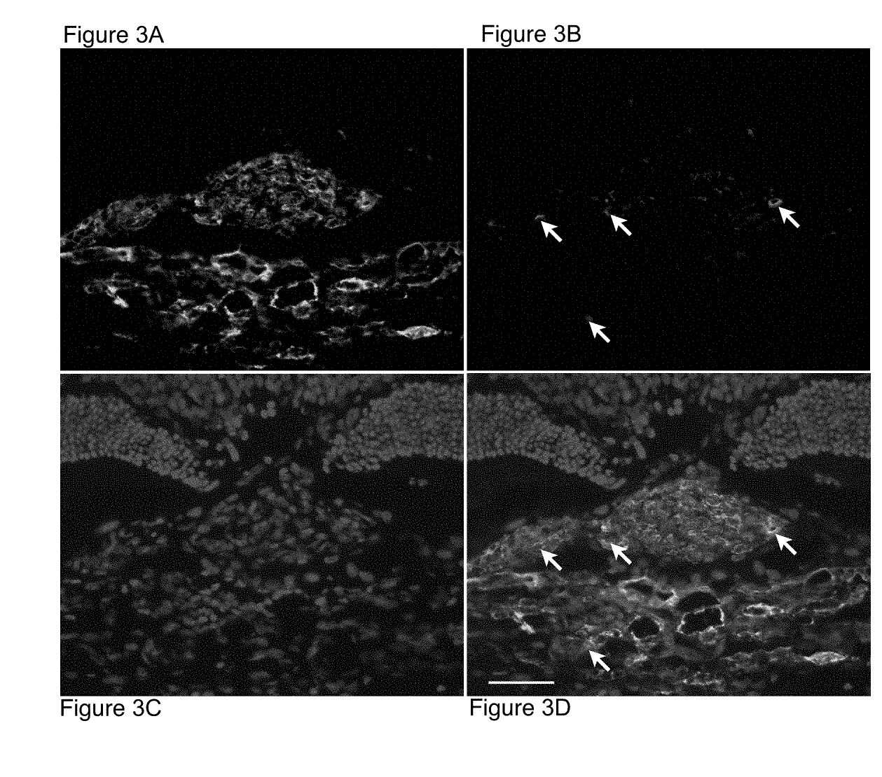

To examine whether VAP-1 contributes to CNV formation, the fundus of Brown Norway Rats was photocoagulated with and without VAP-1 blockade and the size of the CNV in flat mounts of the RPE-choroid complex was quantified (FIG. 4A). In addition, VAP-1 localization in CNV was examined by immunofluorescence staining. The staining for VAP-1 protein was co-localized with isolectin B4 staining in arborizing CNV (FIGS. 3A-3D), suggesting that vascular endothelial cells in CNV lesion also express VAP-1. Furthermore, 7 days after laser injury, the animals treated with VAP-1 inhibitor showed a significant decrease in CNV size (14,536±2175 μm², n=7), compared with vehicle-treated animals (25,026±1586 m², n=9, p<0.01) (FIG. 4B). However, fourteen days after laser injury, the CNV size in the VAP-1 inhibitor-treated animals was not significantly different compared with the vehicle-treated controls (23,992±1437 vs. 26,681±3572 μm², n=10 and 9 eyes, respectively; p=0.5).

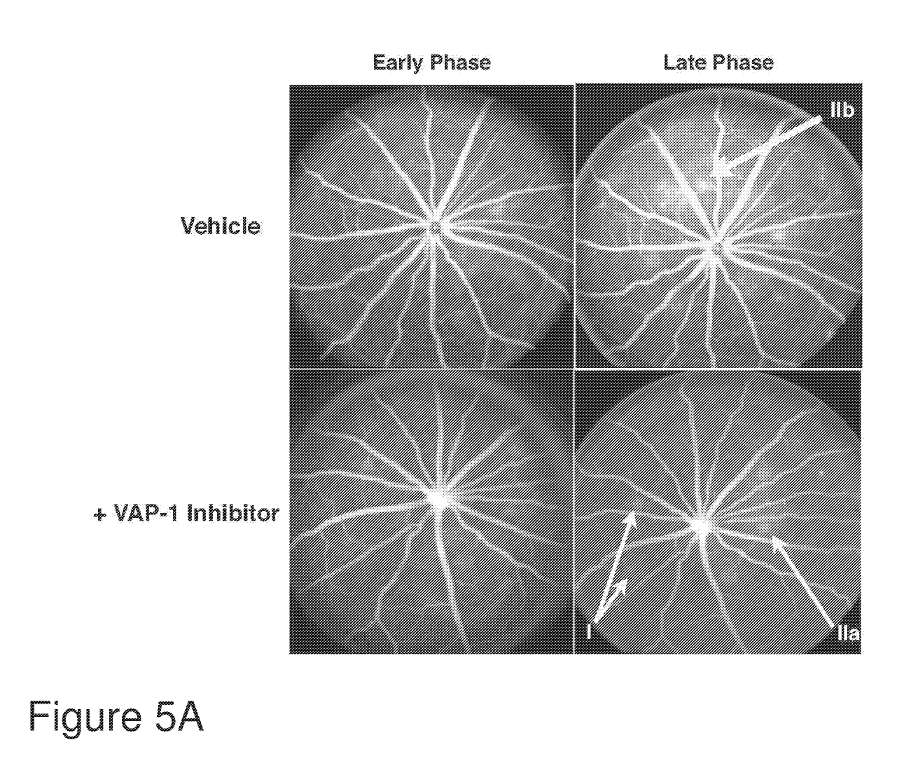

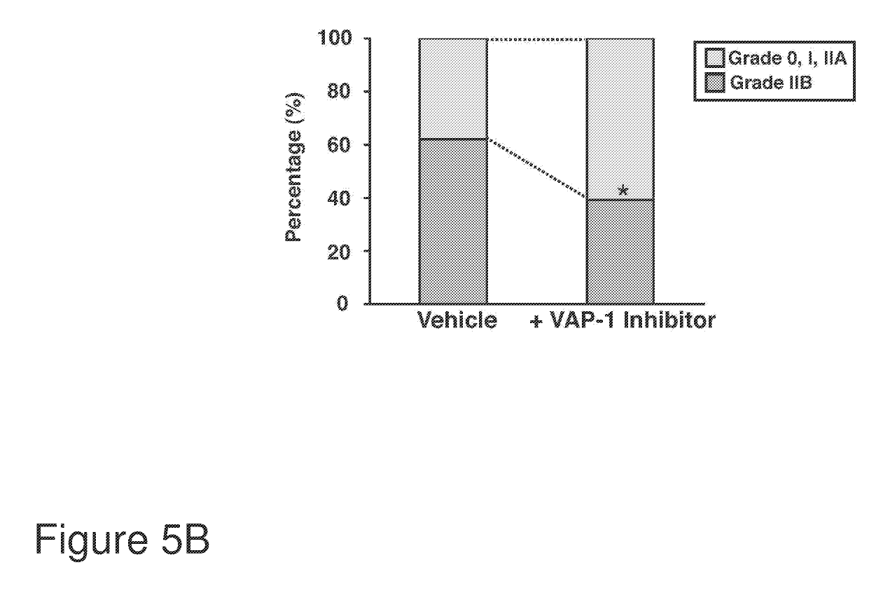

Fluorescent angiography showed that the incidence of the clinically significant CNV lesions, graded as IIB, was significantly decreased in VAP-1 inhibitor-treated animals (41.8%, n=12) in comparison with vehicle-treated animals (64.5%, n=11; p<0.05) (FIGS. 5A and 5B).

Effect of VAP-1 Blockade on Macrophage Infiltration

To investigate whether VAP-1 inhibition affects macrophage infiltration into the CNV lesion, the numbers of ED-1 positive cells in the CNV lesions of animals with or without VAP-1 inhibition were quantified. Macrophages were recruited to the CNV lesion with a peak at day 3 (FIGS. 6A and 6B). In comparison, the number of accumulated macrophages at 3 days after laser injury was significantly reduced, by 41%, with the blockade of VAP-1 (n=4, p<0.05, FIGS. 6A and 6B).

Reduction of Inflammatory Molecules by VAP-1 Blockade

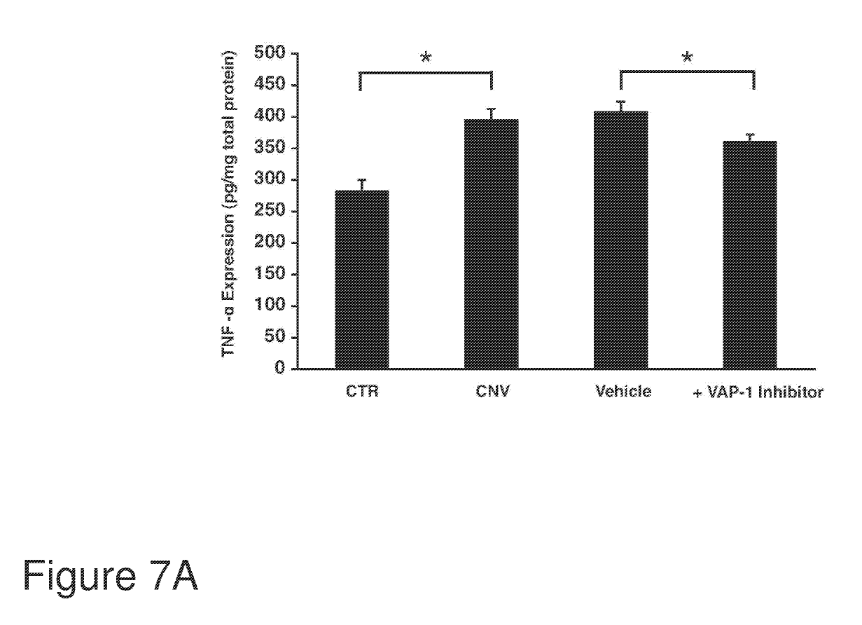

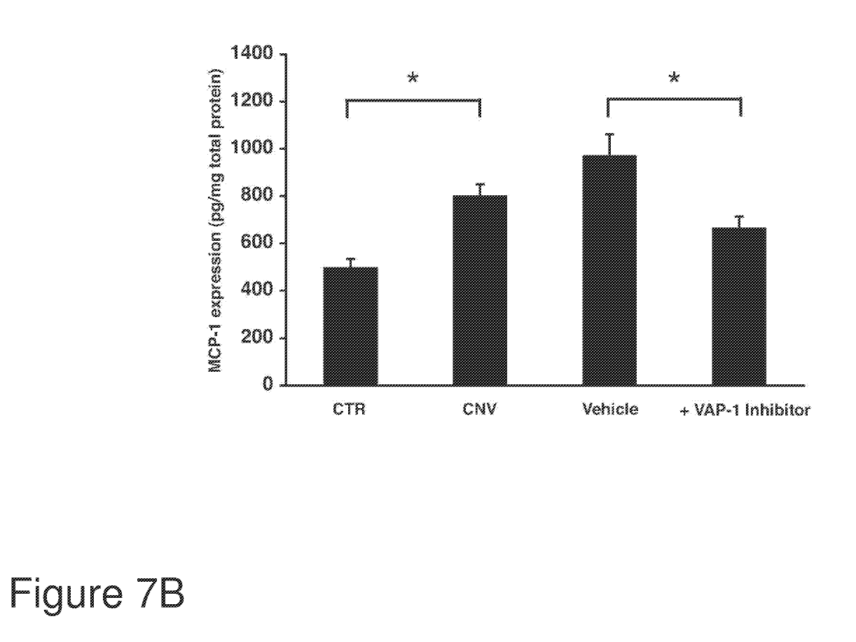

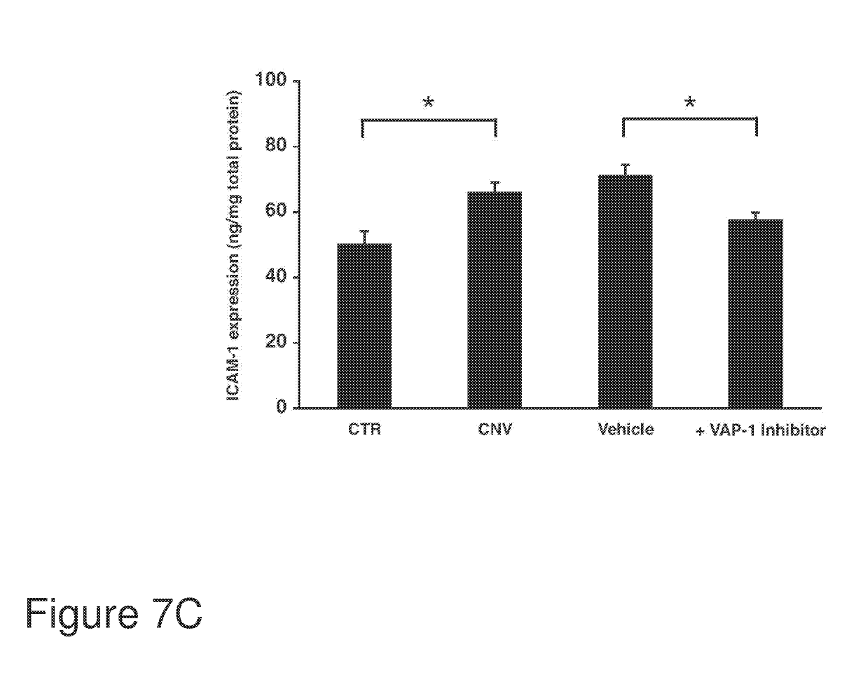

To investigate the mechanisms by which VAP-1 blockade suppresses CNV formation, the levels of the inflammation-associated molecules, TNF-α, MCP-1 and ICAM-1, in the RPE-choroid complex were measured with or without CNV lesions at 3 days after laser irradiation. As compared to protein levels of TNF-α (282±18 pg/mg), MCP-1 (496±38 pg/mg) and ICAM-1 (50±4 ng/mg) in the RPE-choroid complex of normal rats, the protein levels of TNF-α (395±17 pg/mg, p<0.01), MCP-1 (797±53 pg/mg, p<0.01), ICAM-1 (66±3 ng/mg, p<0.01) in the RPE-choroid complex of rats with CNV were significantly increased at 3 days after laser injury (FIGS. 7A-7C). In addition, the protein levels of TNF-α, MCP-1 and ICAM-1 were significantly reduced in the RPE-choroid complex of the laser-treated animals that received the inhibitor compared with the vehicle controls (TNF-α, 407±17 vs. 360±12 pg/mg, p<0.05; MCP-1, 969±93 vs. 662±52 pg/mg p<0.01 ICAM-1, 71±4 vs. 57±2 ng/mg, p<0.01, respectively). There was no statistical difference in the protein levels of the molecules between vehicle-treated and vehicle-untreated CNV animals (TNF-α, p=0.6; MCP-1, p=0.1; ICAM-1, p=0.3, respectively).

d. Discussion

The experiments of this Example investigated the role of VAP-1 in the formation of CNV, an integral component of AMD. The results show constitutively higher levels of VAP-1 expression in the choroid compared to the retina using RT-PCR and immunofluorescence staining. VAP-1 blockade significantly reduced the CNV size seven days after laser injury and macrophage accumulation at the peak of CNV growth, three days after laser injury. These data suggests that the reduction of the CNV formation by VAP-1 blockade may in part be due to suppression of macrophage recruitment.

VAP-1 is a mediator of leukocyte recruitment, particularly of the transmigration step. Recently, VAP-1 has been shown to play a role in acute ocular inflammation. However, whether VAP-1 plays a role in the pathogenesis of AMD was previously unknown. Since inflammatory processes can be involved in the development of AMD, the role of VAP-1 in the formation of CNV, an integral component of AMD, was investigated in the experiments described in this Example. A link between VAP-1 and angiogenesis was discovered.

In addition, constitutively higher levels of VAP-1 expression were found in the choroid as compared to the retina using RT-PCR and immunofluorescence staining. This may in part be due to the higher vascular density in the choroid compared to the retina. The constitutive expression of VAP-1 in the choroid and the retina suggests a role for VAP-1 in leukocyte extravasation in both vascular beds. This suggests that VAP-1 blockade may suppress CNV development through inhibition of inflammatory leukocyte accumulation. Indeed, VAP-1 blockade was shown to significantly reduce the CNV size 7 days after laser injury and the macrophage accumulation at the peak of CNV growth, 3 days after laser injury. This suggests that the reduction of the CNV formation by VAP-1 blockade may in part be due to suppression of macrophage recruitment. However, fourteen days after laser injury, VAP-1 inhibition did not reduce CNV size, suggesting the existence of other VAP-1 independent angiogenic mechanisms that may compensate for the antiangiogenic effect of VAP-1 inhibition seven days after late injury. Inhibition of one angiogenic factor may lead to up-regulation of other factors with functional overlap.

A variety of cytokines, chemokines, and endothelial adhesion molecules play important roles in the pathogenesis of CNV. In the current study, the impact of VAP-1 blockade on the production levels of selected members of these inflammation-associated molecules was investigated. VAP-1 blockade significantly decreased the protein level of the inflammatory cytokine, TNF-α, in the RPE-choroid complexes with CNV. Since macrophages in CNV lesions are a source of TNF-α, it is possible that the inhibition of macrophage infiltration by VAP-1 blockade may underlie the decreased level of TNF-α in the CNV lesions. Interestingly, previous studies show that TNF-α inhibition reduces CNV in an animal model. Furthermore, anti-TNF-α therapy in patients with inflammatory arthritis, who also had AMD, resulted in partial CNV regression and visual acuity improvement. The FA data in the experiments in this Example shows fewer lesions with clinically relevant leakage (Grade IIb) after VAP-1 blockade, compared with the vehicle-treated animals, which suggests that TNF-α reduction through VAP-1 blockade could be an alternate strategy for treatment of AMD.

In addition to TNF-α, VAP-1 blockade also significantly reduced the level of potent macrophage-recruiting chemokine, MCP-1, in the RPE-choroid complex after laser injury. In vitro, TNF-α is known to stimulate RPE cells to produce MCP-1. The data in the experiments described in this Example support a model in which reduced levels of MCP-1 lead to decreased macrophage infiltration. This would cause further reduction of TNF-α release, which in turn would lead to diminished secretion of MCP-1 in RPE cells. VAP-1 blockade may thus interrupt this perpetual cascade of inflammatory events that exacerbate CNV formation at the stage of macrophage transmigration.

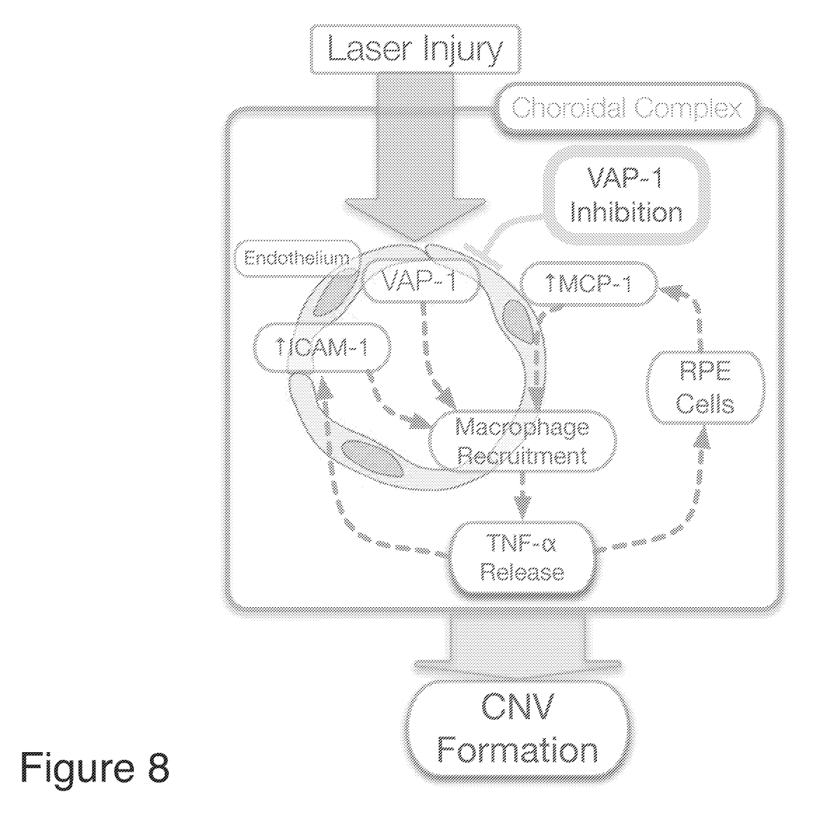

It was also found that VAP-1 blockade significantly reduced the expression of ICAM-1 in choroidal tissues with CNV. ICAM-1, a key endothelial adhesion molecule which regulates leukocyte recruitment, is upregulated in the RPE-choroid complex during CNV formation. Mice deficient for ICAM-1 or its counter receptor, CD18, are known to develop significantly smaller CNV lesions compared with wild-type, suggesting an important role for ICAM-1 in CNV formation. The suppressive effect of VAP-1 blockade on ICAM-1 expression, as observed in this study, is generally consistent with previous data showing that VAP-1 blockade reduces the upregulation of ICAM-1 after LPS stimulation in the retina. The reduction of ICAM-1 expression after VAP-1 blockade in laser-injured eyes may result in lower macrophage infiltration and smaller CNV lesions. Overall, VAP-1 blockade appears to effectively suppress key molecular and cellular components in a cascade leading to CNV formation (FIG. 8). This may be achieved through inhibition of macrophage infiltration and through reduction of the levels of inflammatory cytokines, chemokines and adhesion molecules.

e. Conclusion

In summary, these results show that VAP-1 blockade with the specific inhibitor, Compound II, effectively suppresses CNV. VAP-1 inhibition also reduces macrophage recruitment to the CNV lesions and secretion of inflammatory factors such as MCP-1 and TNF-α in the choroidal tissues. The current results show that VAP-1 inhibitors can be used in the treatment of angiogenic conditions, such as CNV associated with AMD.

### Example 2. VAP-1 Inhibition Suppresses Corneal New Vessel Growth

In this experiment, the role of VAP-1 in corneal angiogenesis and in corneal lymphangiogenesis was investigated. Specifically, the VAP-1 inhibitor, Compound 11 as described above, was administered to animal models of corneal angiogenesis and lymphangiogenesis. Results of this experiment identify VAP-1 as a molecular target in the prevention and treatment of both corneal angiogenesis and corneal lymphangiogenesis, as well as other angiogenic and lymphangiogenic conditions.

a. Methods

Experimental Animals

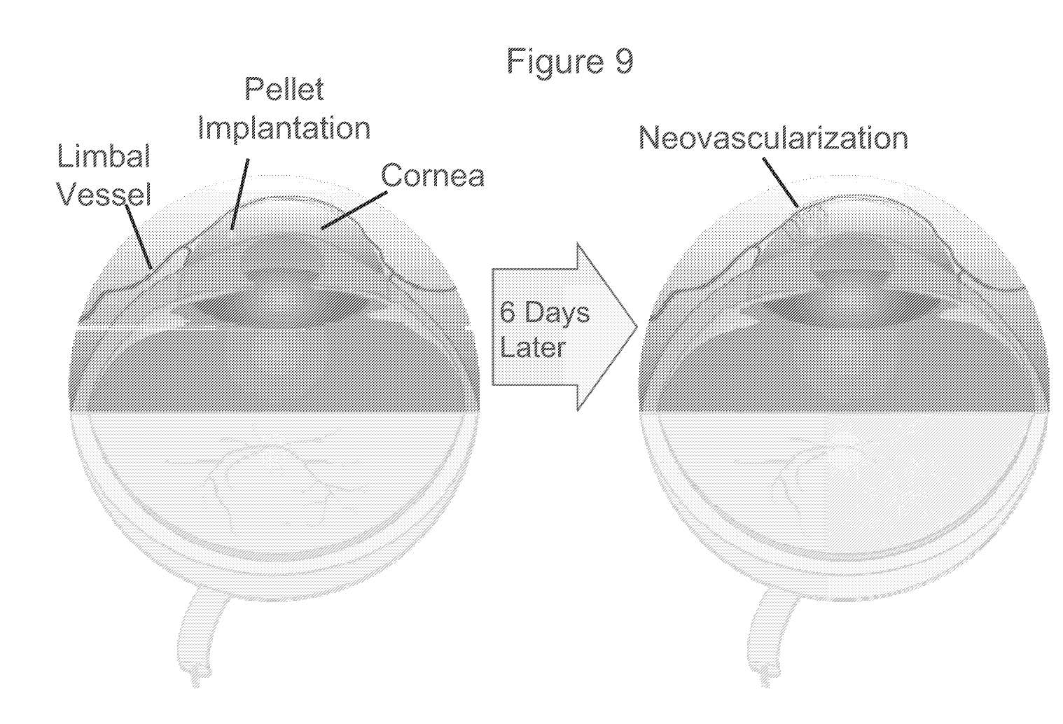

BALB/c mice were anesthetized by intraperitoneal (i.p.) injection of pentobarbital sodium (60 mg/kg). Hydron pellets (0.3 μl) containing 30 ng mouse IL-1β (401-ML; R&D Systems) were prepared and implanted into the corneas. See FIG. 9. Pellets were positioned 1 mm from the corneal limbus. Implanted eyes were treated with Bacitracin ophthalmic ointment (E. Fougera & Co.) to prevent infection.

VAP-1 Inhibition

To block VAP-1, mice received daily i.p. injections of a specific VAP-1 inhibitor, Compound II (R-tech Ueno Ltd., Tokyo, Japan) as described above. A daily dose of 0.3 mg/kg was administered at day 0 and continued until the sixth day after implantation. Two, four and six days after implantation, digital images of the corneal vessels were obtained and recorded using OpenLab software version 2.2.5 (Improvision Inc.) with standardized illumination and contrast and were saved onto disks. The quantitative analysis of new vessel growth in the mouse corneas was performed using Scion Image software (version 4.0.2; Scion Corp.).

Whole-Mount Immunohistochemistry

Eyes were enucleated and fixed with 4% paraformaldehyde for one hour at 4° C. For whole-mount preparation, the corneas were exposed by removing other portions of the eye (i.e. iris, sclera, retina, and conjunctiva). After washing with PBS, tissues were placed in methanol for 20 minutes. Tissues were incubated overnight at 4° C. with antibodies for CD31 (1:25, 550274; BD Pharmingen, San Diego, Calif.), LYVE-1 (4 μg/ml, 103-PA50AG; RELIAtech, Germany), VAP-1 (1:40, sc-13743; Santa Cruz) or VAP-1 (1:20, HM1094; Hycult biotechnology, Netherlands) diluted in PBS containing 10% goat serum and 1% Triton X-100. Tissues were washed four times in PBS followed by incubation with FITC-conjugated goat anti-rat Ab (1:100, AP136F; Chemicon International), Alexa Fluor 647 goat anti-rabbit Ab (1:100, A21244; Invitrogen) or Alexa Fluor 647 chicken anti-goat Ab (1:100. A21469; Invitrogen) overnight at 4° C. Radial cuts were then made in the peripheral edges of the tissue to allow flat mounting on a glass slide in mounting medium (Vectashield; Vector Laboratories).

Immunostaining

Mice were sacrificed under deep anesthesia with pentobarbital sodium (60 mg/kg i.p.). The eyes were harvested, snap-frozen in optimal cutting temperature (OCT) compound (Sakura Finetechnical) and 10 μm sections were prepared, air-dried and fixed in cold acetone for 10 min. The sections were blocked with nonfat dried-milk (M7409; Sigma) for 10 minutes and stained with anti-CD11b mAb (1:100, 550282; BD Pharmingen), anti-Gr-1 mAb (1:100, 550282; BD Pharmingen) or anti-F4/80 mAb (1:100, MCA497G; Serotec). After an overnight incubation, sections were washed and stained for 20 min. with secondary Abs, FITC-conjugated goat anti-rat (1:100, AP136F; Chemicon International).

b. Results and Discussion

VAP-1 Blockade Inhibits IL-1β-Induced Angiogenesis

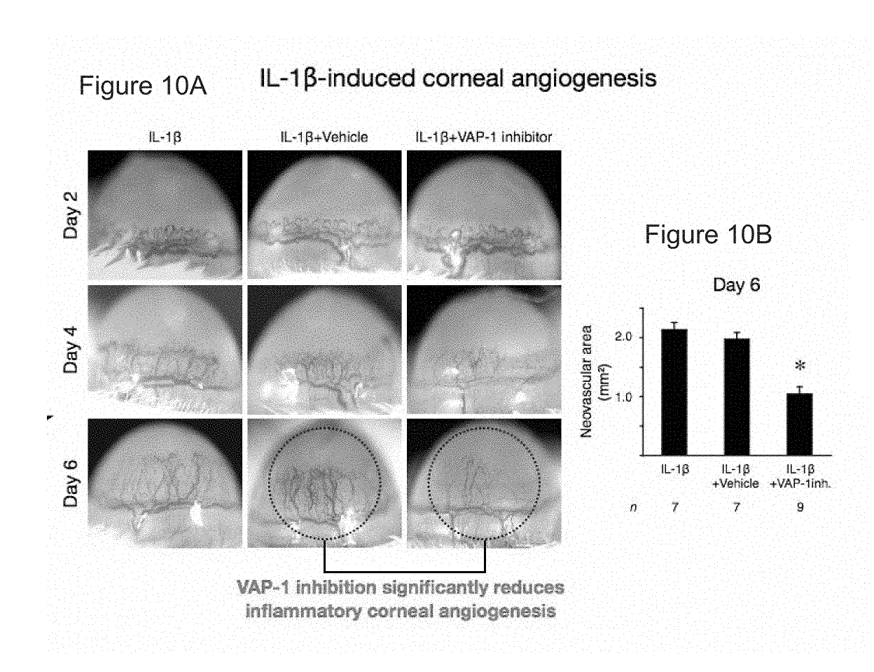

It was found that i.p. administration of a VAP-1 inhibitor significantly reduced corneal angiogenesis. Specifically, FIG. 10A shows digital images of the corneal vessels at 2, 4, and 6 days after inducing corneal angiogenesis in mice using after IL-1β. In control mice exposed to IL-1β alone or IL-1β+vehicle, a significant increase in neovascularization was observed at day 6. However, in the mice treated with IL-1β+VAP-1 inhibitor, there was a significant reduction in inflammatory corneal angiogenesis. Quantitatively, as shown in the chart in FIG. 10B, the neovascular area at day 6 in the IL-1β+VAP-1 inhibitor mice was about half that of the neovascular area of the control mice exposed to IL-1β alone or IL-1β+vehicle.

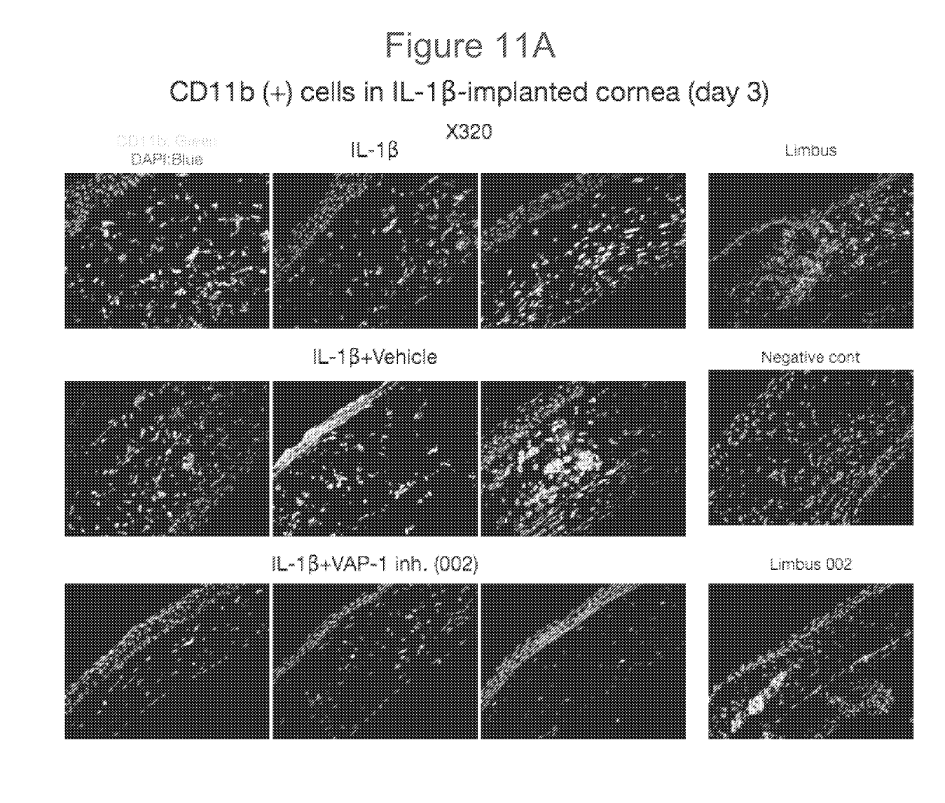

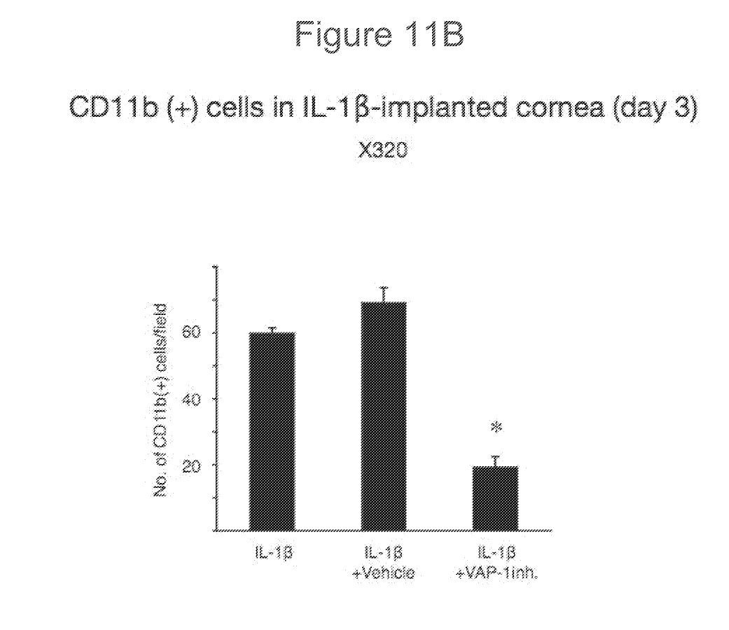

To examine the effect of VAP-1 inhibition on leukocyte infiltration, the infiltration of CD11b(+) cells was compared between corneas of animals treated with a VAP-1 inhibitor and corneas of untreated animals. FIGS. 11A and 11B depict the impact of VAP-1 inhibition on CD11b(+) cells in IL-1β-induced corneal angiogenesis at 3 days after pellet implantation. FIG. 1A is a set of photomicrographs showing CD11b(+) cells in corneas treated with IL-1β, IL-1β+vehicle, or IL-1β+VAP-1 inhibitor. FIG. 11B is a graph comparing the number of CD11b(+) cells appearing in IL-1β-implanted cornea with and without VAP-1 inhibition, at 3 days after pellet implantation. The comparison indicates that infiltration of CD11b(+) cells was effectively inhibited by systemic administration of the VAP-1 inhibitor.

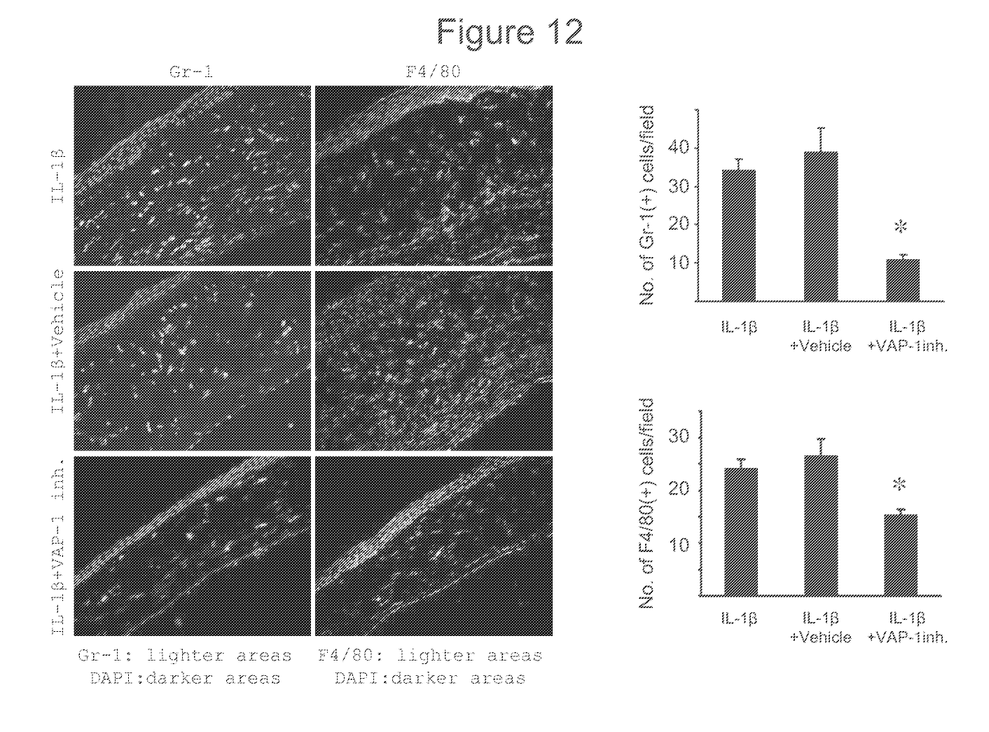

To examine which population of leukocytes was affected by VAP-1 blockade, the number of Gr-1(+) cells (indicative of neutrophils and macrophages) and F4/80(+) cells (indicative of monocytes and macrophages) in IL-1β-implanted corneas was examined. FIG. 12 depicts the impact of VAP-1 inhibition on Gr-1(+) cells and F4/80(+) cells in IL-1β-induced corneal angiogenesis. The left side of FIG. 12 is a set of photomicrographs showing staining of Gr-1(+) cells (left column) and F4/80(+) cells (right column) in corneas treated with IL-1β, IL-1β+vehicle, or IL-1β+VAP-1 inhibitor. The right side of FIG. 12 shows graphs comparing the number of Gr-1(+) cells and F4/80(+) cells, respectively, appearing in IL-1β-implanted cornea with and without VAP-1 inhibition, following implantation. Both the number of Gr-1(+) cells and F4/80(+) cells in VAP-1 inhibitor-treated cornea were less than in vehicle-treated cornea or untreated cornea. This result is consistent with a number of studies which have suggested that leukocytes play an important role in corneal angiogenesis. Specifically, if CD11b(+) cells are a factor in corneal angiogenesis, then the mechanism by which VAP-1 blockade inhibits angiogenesis may include inhibition of CD11b(+) cells, as seen in these results.

VAP-1 Blockade Inhibits IL-1β-Induced Lymphangiogenesis

It was found that i.p. administration of a VAP-1 inhibitor reduced corneal lymphangiogenesis. Specifically, FIG. 13 shows a set of photographs of corneal tissue samples following induction of corneal lymphangiogenesis with IL-1β and treatment with vehicle (IL-1β+Vehicle) or VAP-1 inhibitor (IL-1β+VAP-1 inh). Anti-LYVE-1 stain identifies lymphatic vessels. As shown in FIG. 13, VAP-1 inhibitor reduced growth of lymphatic vessels in a lymphangiogenesis model.

VAP-1 Expression in Non-Inflamed Versus Inflamed Corneas

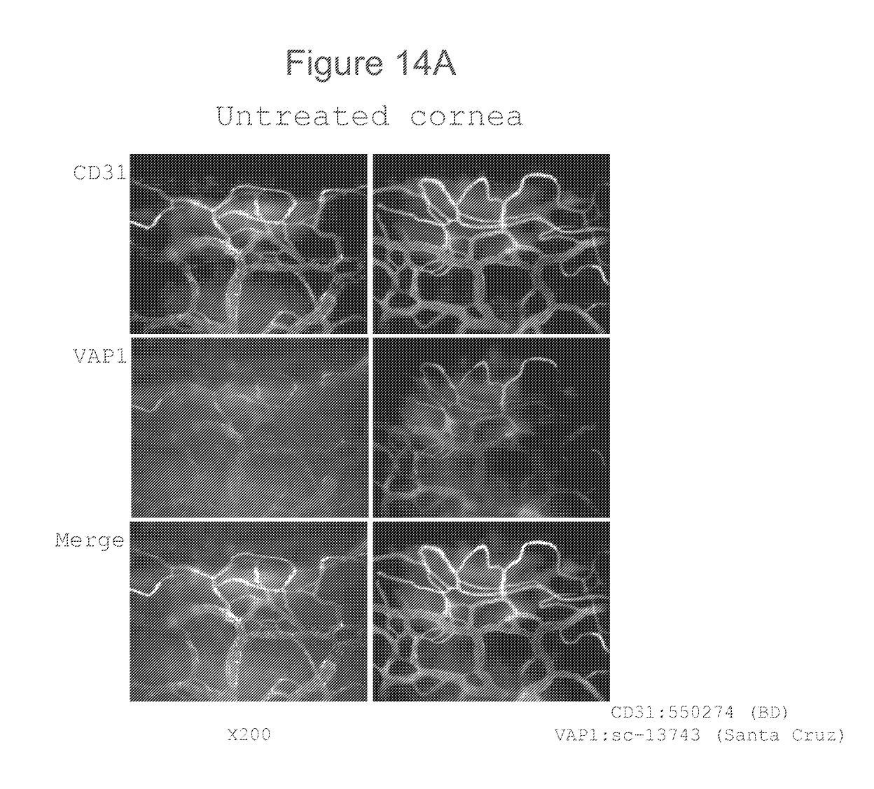

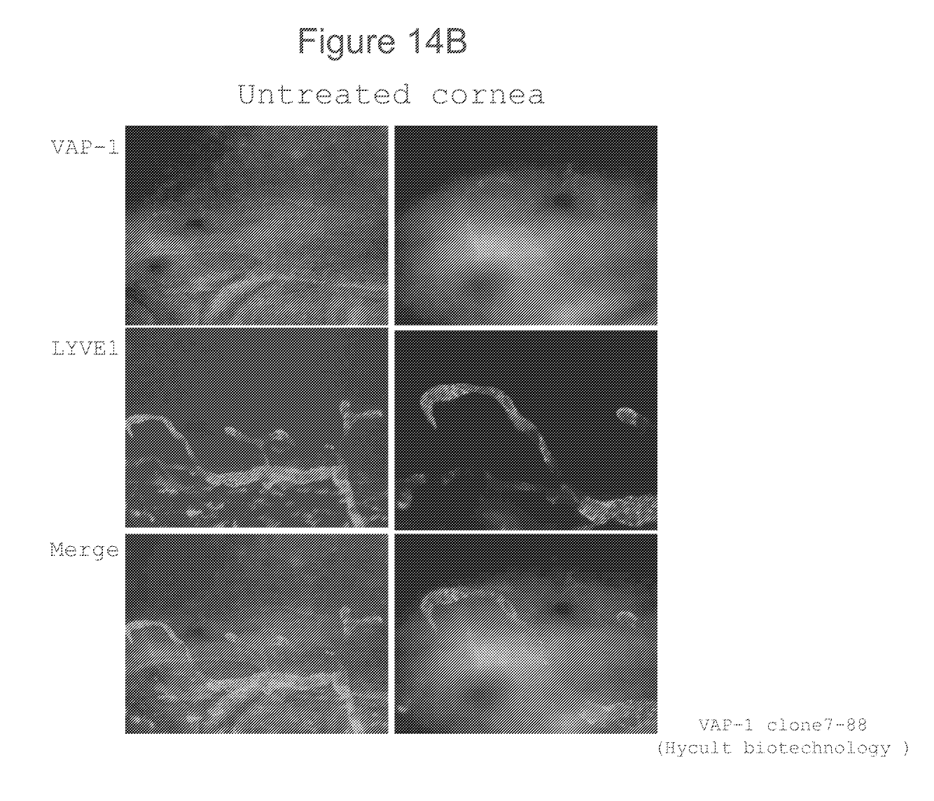

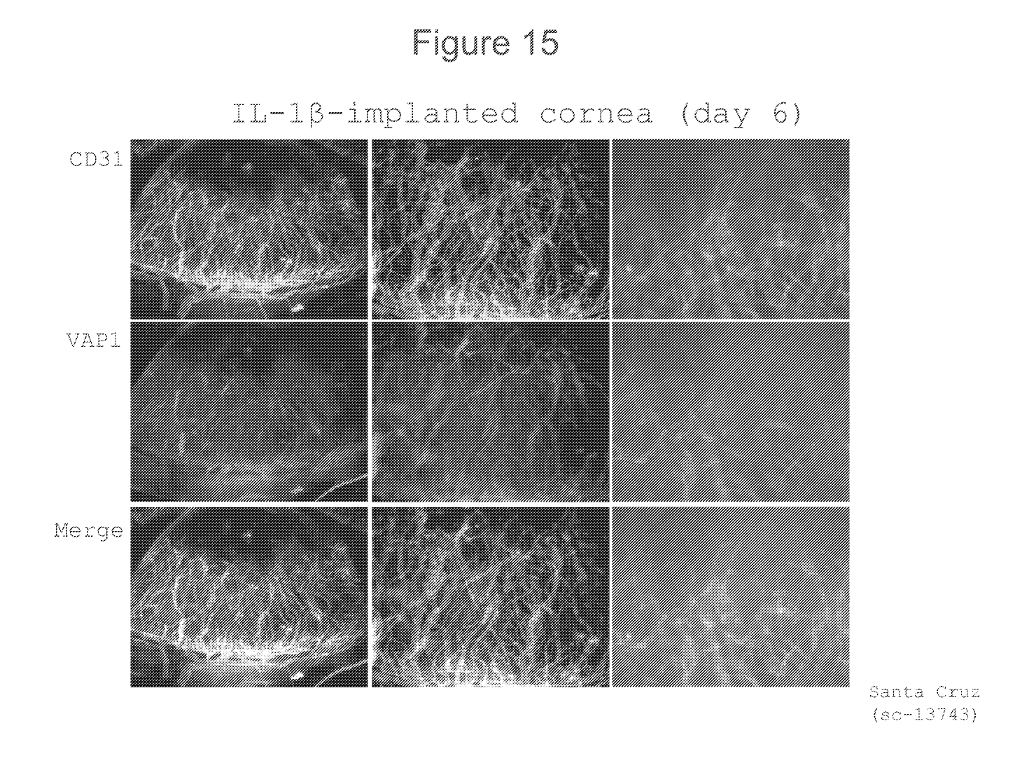

VAP-1 expression in inflamed and non-inflamed corneas was also compared. Immunohistochemistry showed that VAP-1 was expressed in blood vessels in both inflamed and non-inflamed corneas (with and without IL-1β implantation). FIG. 14A shows a set of photographs of untreated corneal tissue (no IL-1β treatment). Samples in the top two photographs were stained with anti-CD31 to identify endothelial cells in blood vessels. Samples in the middle two photographs were stained with anti-VAP-1 to identify the presence of VAP-1. The bottom two photographs shows merger of the two photographs above it and indicate that VAP-1 is expressed on quiescent blood vessels. FIG. 15 shows a set of photographs of corneal tissue that from corneas treated with IL-1β to induce angiogenesis. Samples in the top three photographs were stained with anti-CD31 to identify endothelial cells in blood vessels. Samples in the middle three photographs were stained with anti-VAP-1 to identify the presence of VAP-1. The bottom three photographs shows merger of the two photographs above it and indicates that VAP-1 is expressed on angiogenic blood vessels.

However, VAP-1 did not appear to be expressed in lymphatic vessels in un-inflamed cornea (no IL-1β implantation). FIG. 14B also shows a set of photographs of untreated corneal tissue (no IL-1β treatment). Samples in the top two photographs were stained with anti-VAP-1 to identify the presence of VAP-1. Samples in the middle two photographs were stained with anti-LYVE-1 to identify lymphatic vessels. The bottom two photographs shows merger of the two photographs above it and indicate that VAP-1 is not expressed on quiescent lymphatic vessels.

c. Conclusion

In summary, these results show that VAP-1 blockade with the specific inhibitor, Compound II, effectively suppresses corneal angiogenesis as compared untreated controls. VAP-1 inhibition also reduces CD11b(+) cells in the cornea and limbus.

These results also show that VAP-1 blockade with the specific inhibitor, Compound II, effectively suppresses corneal lymphangiogenesis as compared untreated controls. Accordingly, the current results show that VAP-1 inhibitors can be used in the treatment of corneal angiogenesis and in the treatment of corneal lymphangiogenesis, as well as other angiogenic and lymphangiogenic conditions.

### Example 3. VAP-1 Inhibition Suppresses Metastatic Tumor Growth

The following experiment describes a method for observing the ability of a VAP-1 inhibitor to suppress metastatic tumor growth.

a. Method

Animals with a Lewis lung carcinoma tumor between 600-1200 mm³ in size are sacrificed and the skin overlying the tumor is cleaned with betadine and ethanol. In a laminar flow hood, the tumor tissue is excised under aseptic conditions. A suspension of tumor cells in 0.9% normal saline is made by passage of viable tumor tissue through a sieve and a series of sequentially smaller hypodermic needles of diameter 22- to 30-gauge. The final concentration is adjusted to 1×10⁷ cells/ml and the suspension is placed on ice. After the site is cleaned with ethanol, the subcutaneous dorsa of mice in the proximal midline are injected with 1×10⁶ tumor cells in 0.1 ml of saline.

When tumors reach 1500 mm³ in size, the tumors are surgically removed from the mice. The incision is closed with simple interrupted sutures. From the day of operation, mice receive daily injections of a VAP-1 inhibitor or a saline control. When the control mice become sick from metastatic disease (i.e., after 13 days of treatment), all mice are sacrificed and autopsied. Lung surface metastases are counted by means of a stereomicroscope at 4× magnification.

b. Expected Results

It is expected that mice treated with the VAP-1 inhibitor as compared to control mice treated with saline show significantly diminished metastasized tumor growth in the lungs.

### Example 4. VAP-1 Inhibition Suppresses Primary Tumor Growth

The following experiment describes a method for observing the ability of a VAP-1 inhibitor to suppress primary tumor growth.

a. Methods

Mice are implanted with Lewis lung carcinomas as described in Example 3. Tumors are measured with a dial-caliper and tumor volumes are determined, and the ratio of treated to control tumor volume (T/C) is determined for the last time point. After tumor volume is 100-200 mm³ (0.5-1% of body weight), mice are randomized into two groups. One group receives the VAP-1 inhibitor injected once daily. The other group receives comparable injections of the vehicle alone. The experiments are terminated and mice are sacrificed and autopsied when the control mice begin to die.

b. Expected Results

It is expected that the growth of Lewis lung carcinoma primary tumors is inhibited by the administration of the VAP-1 inhibitor as compared to the saline control.

### Example 5. Localization of VAP-1 in the Human Eye

To further understand the role of VAP-1 in angiogenic disorders, such as ocular angiogenic disorders, the expression of VAP-1 in the human eye was investigated. This example shows that, in the human, VAP-1 is localized to areas consistent with the data shown in Examples 1 and 2 as well as its role as a therapeutic target for ocular angiogenic conditions described herein.

Briefly, five micrometer thick sections were generated from human ocular tissues embedded in paraffin. VAP-1 localization was investigated by immunohistochemistry. Sections were incubated overnight with primary monoclonal antibodies against VAP-1 (5 μg/ml), smooth muscle actin (1 μg/ml), CD31, or isotype-matched IgG at 4° C. Subsequently, a secondary monoclonal antibody was used for 30 minutes at room temperature, followed by use of the Dako Envision+HRP (AEC) System (available from Dako North America, Inc., Carpenteria, Calif.) for signal detection. The stained sections were examined using light microscopy, and the signal intensity was quantified by two masked evaluators and graded into four discrete categories.

In all examined ocular tissues, VAP-1 staining was confined to the vasculature. VAP-1 labeling showed the highest intensity in both arteries and veins of neuronal tissues, retina and optic nerve, and the lowest intensity in the iris vasculature. Scleral and choroidal vessels showed moderate staining for VAP-1. VAP-1 intensity was significantly higher in the arteries compared to veins. Furthermore, VAP-1 staining in arteries co-localized with SM-actin staining, suggesting expression of VAP-1 in smooth muscle cells or, potentially, pericytes.

Immunohistochemistry revealed constitutive expression of VAP-1 in human ocular tissues. VAP-1 expression is exclusive to the vasculature with arteries showing significantly higher expression than veins. Furthermore, VAP-1 expression in the ocular vasculature is heterogeneous, with the vessels of the optic nerve and the retina showing highest expressions. These results suggest VAP-1 is a relevant molecule in ocular vascular and inflammatory diseases in humans.

a. Methods

Tissue Samples

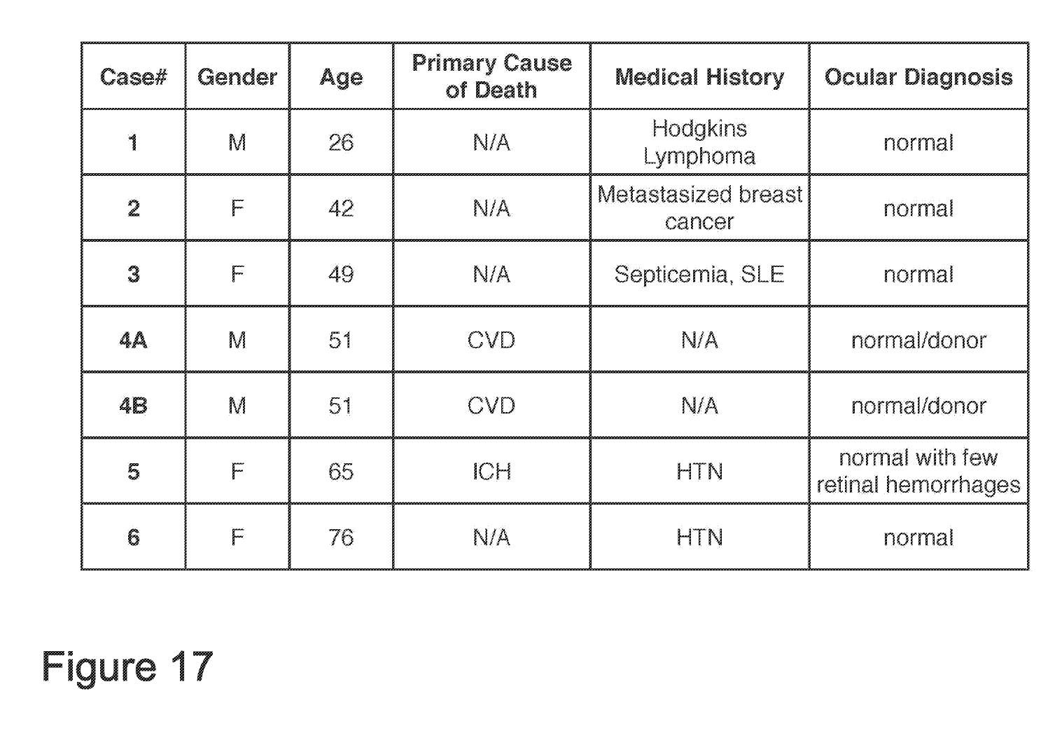

Paraffin-embedded blocks of normal human ocular tissues were obtained from the Massachusetts Eye and Ear Infirmary's (MEEI) stored archives of samples. FIG. 17 describes each of the sample donors. All materials were used in accordance with the protocol approved by the Institutional Review Board (IRB) of the MEEI and in accordance with the Declaration of Helsinki.

Immunohistochemistry

VAP-1 tissue localization was examined in paraffin-embedded sections of human eyes. The slides were dewaxed and hydrated through exposure with graded alcohols (100% then 95%) followed by water. Endogenous peroxidase activity was then blocked by placing the sections in 0.3% hydrogen peroxide (Sigma Aldrich, St. Louis, Mo., US) for 15 minutes, and non-specific binding was blocked by subsequently placing the sections in 10% normal goat serum (Invitrogen, CA) for 1 hour. Subsequently, the sections were reacted with primary monoclonal antibodies (mAb) against either VAP-1 (5 μg/ml; BD Biosciences, Franklin Lakes, N.J.), endothelial CD31 (Dako North America, Inc., Carpinteria, Calif.) or smooth muscle actin (1 μg/ml; Sigma, St. Louis, Mo.) at 4° C. overnight. For CD31 staining, deparaffinized sections were heated in a water bath at 97° C. for 10 minutes.

Thereafter, the sections were incubated for 30 minutes at room temperature with Envision system secondary antibodies against mouse IgG (Dako North America, Inc., Carpinteria, Calif.). For signal detection, the Dako Envision+HRP (AEC) System was used according to the manufacturer's protocol. Finally, sections were counterstained with hematoxylin. Photomicrographs were taken with a digital high sensitivity camera (Hamamatsu, ORCA-ER C4742-95, Japan). As a negative control, the primary antibodies were replaced with non-immune mouse IgG (Dako North America, Inc., Carpinteria, Calif.).

Data and Statistical Analysis

Histological sections were examined under light microscopy and graded by two independent experimenters. VAP-1 signal intensity was judged as: no (“−”), moderate (“+”), and strong (“++”) staining. To compare the results from different groups, the grades given by the observers were averaged for each eye and plotted as 0, 1 and 2, respectively. For statistical analysis, the results were divided into two groups (0 or higher). A Chi-square test was used to calculate the degree of confidence with which the data supports the null hypothesis. Probability values (p) less than 0.05 were considered statistically significant.

b. Results

Exclusive Expression of VAP-1 in the Vasculature of the Human Eye

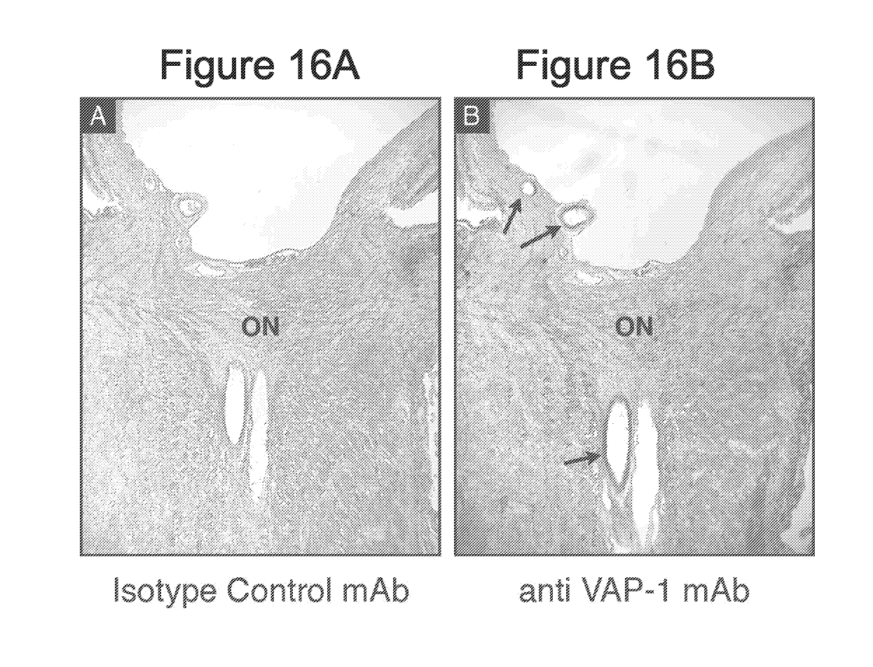

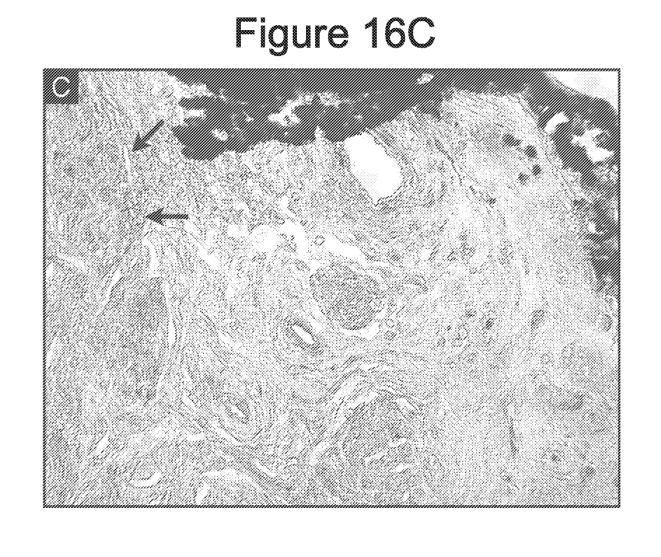

To determine VAP-1 expression in the human eye, immunohistochemistry was performed on normal human ocular tissues (n=7). In various ocular tissues, VAP-1 specific signal was almost exclusively confined to the vasculature as compared to nonimmune isotype control. Particularly, VAP-1 was observed in the inner and medial layers, but not the outer adventitial layer, of the main branches of the ophthalmic artery. In contrast, small capillaries did not show VAP-1 expression (FIGS. 16A and 16B). Outside of the vessels, VAP-1 expression also was observed in the smooth muscle cells of the ciliary body (FIG. 16C) while no VAP-1 staining was observed in the retinal pigment epithelium (RPE) layer of any of the eyes.

VAP-1 Distribution in Normal Human Ocular Tissues

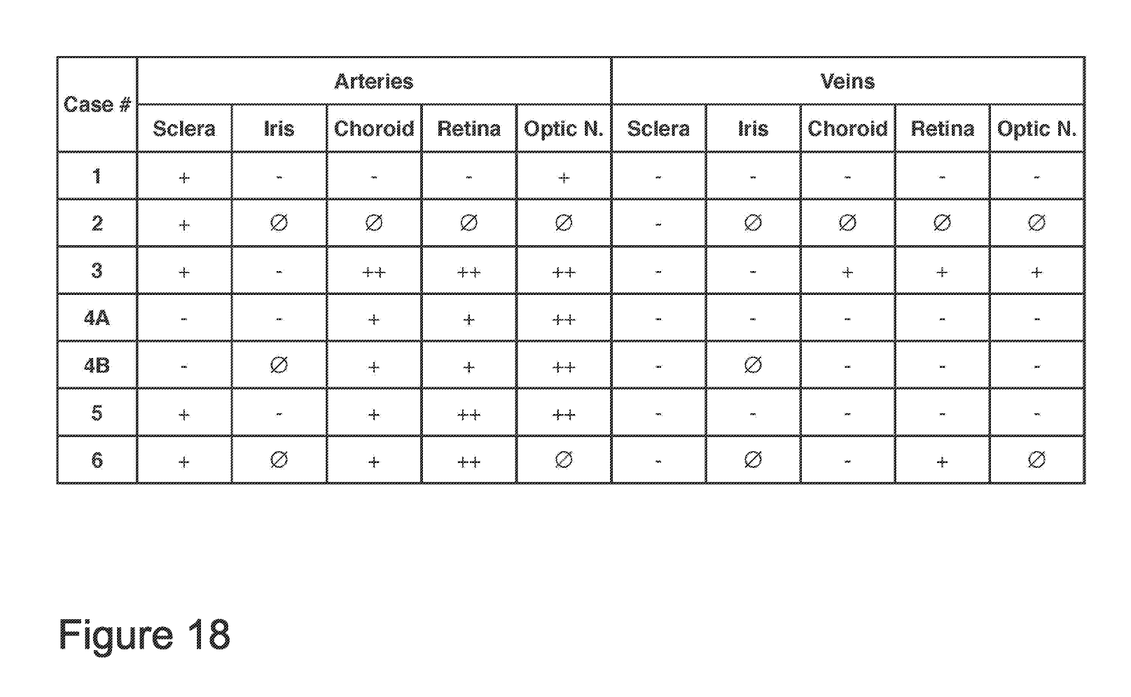

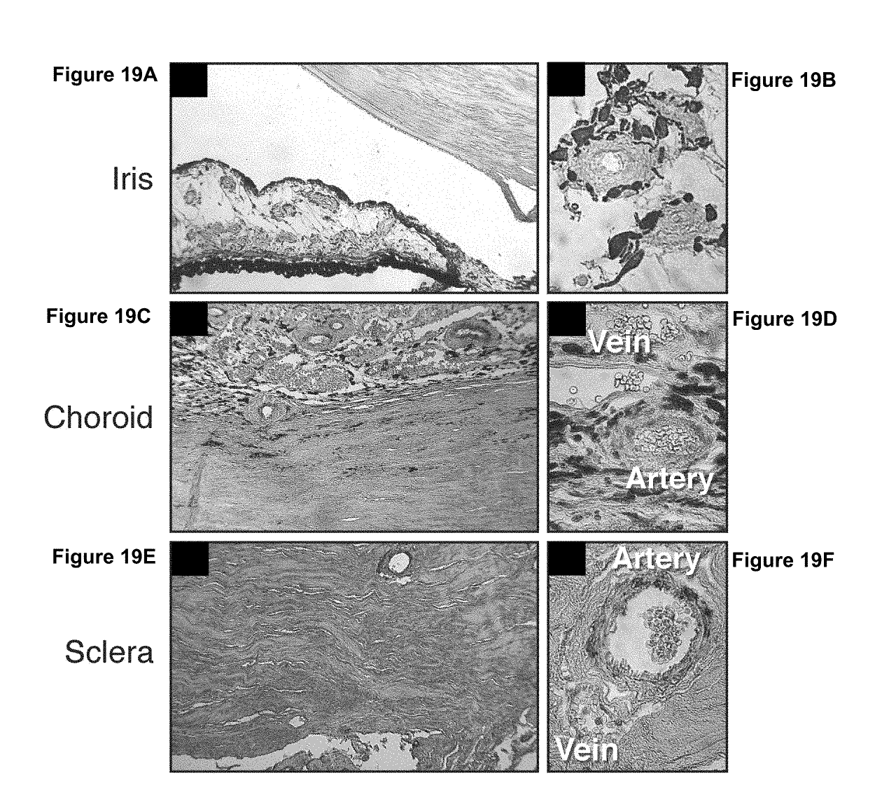

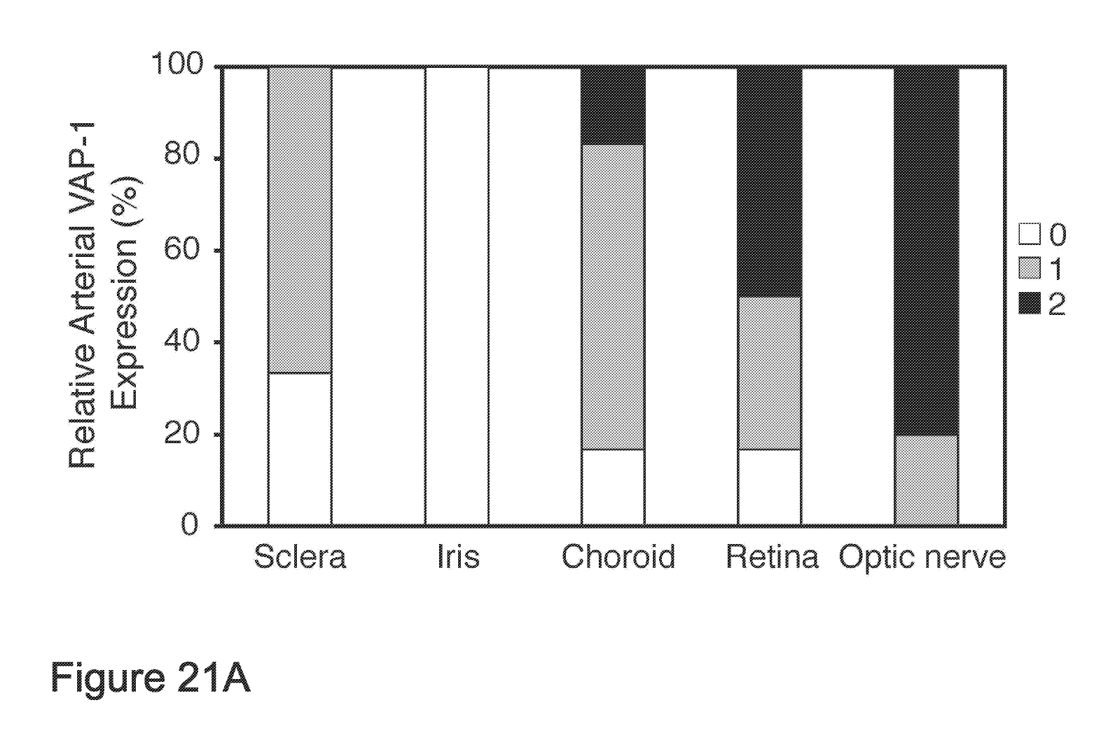

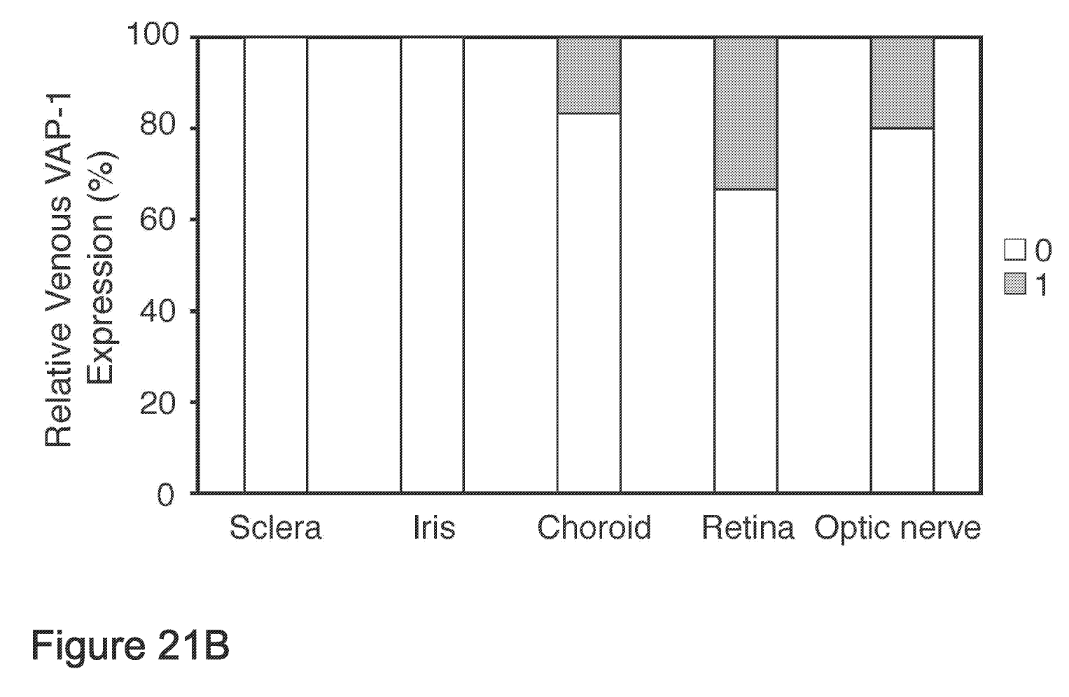

To compare the vascular VAP-1 expression in different ocular tissues, VAP-1 signal intensity was quantified by grading (FIG. 18). No appreciable staining for VAP-1 was observed in the iris vessels, both arteries and veins (n=4) (FIGS. 19A and 19B). Compared with the iris arteries, arteries of the choroidal (n=6) and scleral (n=7) tissues (n=7) showed significantly higher VAP-1 staining (p<0.05) (FIGS. 19C-19F), and arteries of neuronal tissues, the retina (n=6) and optic nerve (n=7), showed the most prominent staining (p<0.05 and p<0.01, respectively) (FIGS. 20A-20C, quantified in FIG. 21A). In contrast, no significant difference was observed in venular VAP-1 expression of all groups (p>0.1; quantified in FIG. 21B).

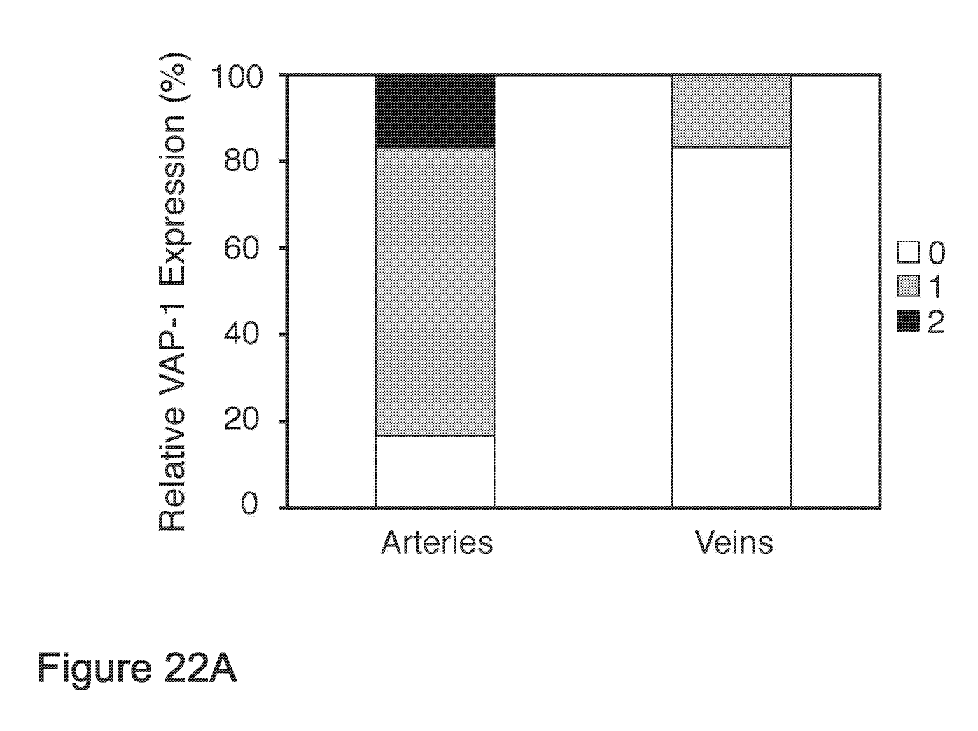

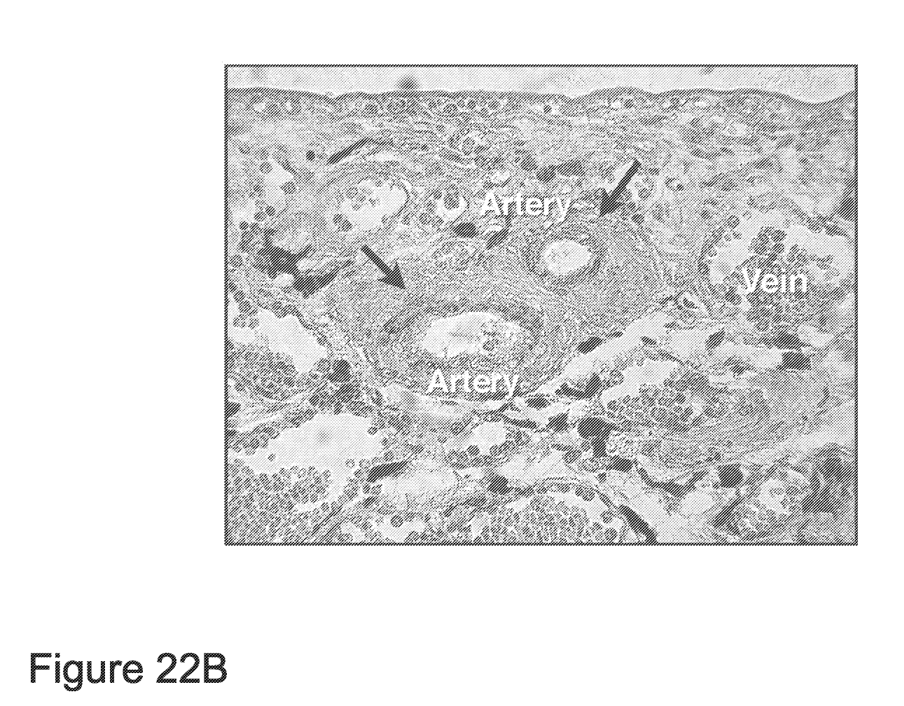

VAP-1 expression also was compared between arteries and veins. VAP-1 expression was significantly higher in arteries than veins in all examined tissues (p<0.05), except for the iris vessels (FIGS. 22A and 22B).

Localization of VAP-1 to Both Vascular Endothelial and Smooth Muscle Cells

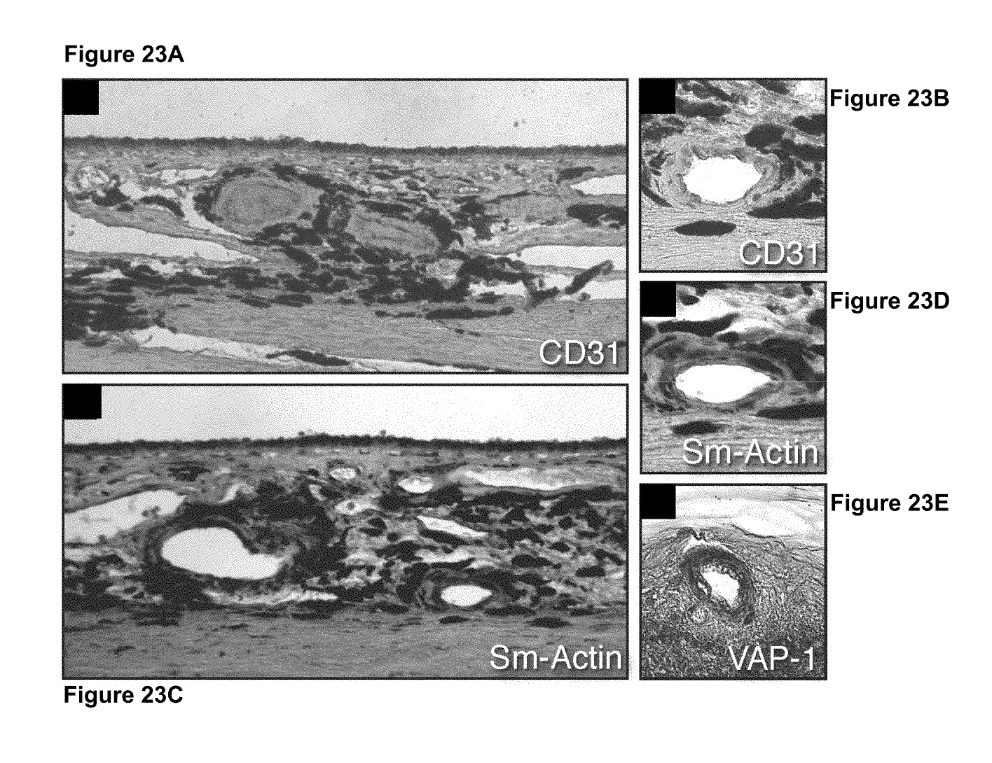

To further investigate the cellular distribution of VAP-1, co-immunostaining of CD31, a marker for endothelial cells, and sm-actin, a marker for smooth muscle cells, was performed. In line with previous studies in various other human tissues (Jaakkola, K. et al. (1999) AM J PATHOL 155:1953-1965) in the eye, VAP-1 co-localized both in endothelial and smooth muscle cells (FIGS. 23A-23E).

c. Discussion

In this series of experiments, the distribution pattern of VAP-1 in human ocular tissues was determined. In the eye, VAP-1 is exclusively expressed in the vasculature. Arteries show significantly higher levels of VAP-1 staining than veins, suggesting a specialized role for this molecule in diseases with primary arterial involvement. The difference between arterial and venous expression may be relevant in the pathogenesis of diabetic retinopathy, where capillary non-perfusion, due to leukocyte plugging at the capillary entrance has been postulated as an important component (Miyamoto et al. (1999) PROC NATL ACAD SCI USA 96:10836-10841; Miyamoto el al. (1999) SEMIN OPHTHALMOL 14:233-239; Schroder (1991) AM J PATHOL 139:81-100). Most adhesion molecules, such as ICAM-1 or P-selectin, which lead to leukocyte adhesion in postcapillary venules, would not sufficiently explain this phenomenon (Miyamoto et al. PROC NATL ACAD SCI USA, supra). Furthermore, the higher expression of VAP-1 in arteries together with the specialized role of this molecule for leukocyte transmigration confirms this molecule as a target in ocular diseases, such as ocular angiogenic conditions.

These studies also indicate that in addition to the endothelium, smooth muscle cells also express VAP-1. Since arteries have both endothelial and smooth muscle cells, while veins have only endothelial cells, this might in part explain the higher level of VAP-1 expression in arteries compared to veins. Furthermore, heterogeneity in the vascular expression of VAP-1 was found within the various regions of the eye. While vessels of the optic nerve head expressed highest amounts of the molecule, the iris vessels did not show detectable expression. The broad expression of VAP-1 in the posterior section of the eye suggests an involvement of the molecule in ocular diseases, such as age-related macular degeneration and diabetic retinopathy in humans.

The experiments in this Example show constitutive expression of VAP-1 in humans, show its presence in human tissues consistent with its role as a therapeutic target for ocular angiogenic conditions described herein, and confirm its role in human angiogenic conditions, such as ocular angiogenic conditions.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

### INCORPORATION BY REFERENCE

The entire disclosure of each of the patent documents and scientific publications disclosed hereinabove is expressly incorporated herein by reference for all purposes.

What is claimed is:

1. A method for treating an angiogenic condition, the method comprising: administering a VAP-1 inhibitor to a subject in an amount sufficient to inhibit angiogenesis.

2. The method of claim 1, further comprising performing photodynamic therapy.

3. The method of claim 1, further comprising administering a VEGF inhibitor.

4. The method of claim 1, wherein the VAP-1 inhibitor is administered locally.

5. The method of claim 1, wherein the condition is selected from the group consisting of scar formation, tissue repair, wound healing, athlerosclerosis, and arthritis.

6. The method of claim 1, wherein inhibition of angiogenesis comprises blood vessel regression or inhibition of blood vessel formation.

7-8. (canceled)

9. A method for treating an ocular angiogenic condition, the method comprising:
administering a VAP-1 inhibitor to a subject in an amount sufficient to inhibit angiogenesis of the eye.

10. The method of claim 9, wherein the ocular angiogenic condition comprises unwanted choroidal neovasculature and the VAP-1 inhibitor is administered to the subject in an amount sufficient to inhibit unwanted choroidal neovasculature.

11. The method of claim 10, wherein the subject has age-related macular degeneration.

12. The method of claim 10, wherein inhibition of unwanted choroidal neovasculature comprises blood vessel regression or inhibition of blood vessel formation.

13. The method of claim 9, wherein the ocular angiogenic condition comprises corneal angiogenesis and the VAP-1 inhibitor is administered to the subject in an amount sufficient to inhibit corneal angiogenesis.

14. The method of claim 13, wherein inhibition of corneal angiogenesis comprises blood vessel regression or inhibition of blood vessel formation.

15. A method for treating a lymphangiogenic condition, the method comprising: administering a VAP-1 inhibitor to a subject in an amount sufficient to inhibit lymphangiogenesis.

16. The method of claim 15, further comprising performing photodynamic therapy.

17. The method of claim 15, further comprising administering a VEGF inhibitor.

18. The method of claim 15, wherein the VAP-1 inhibitor is administered locally.

19. The method of claim 15, wherein the condition is selected from the group consisting of scar formation, tissue repair, wound healing, rheumatoid arthritis, and organ transplantation.

20. The method of claim 15, wherein inhibition of lymphangiogenesis comprises lymph vessel regression or inhibition of lymph vessel formation.

21-23. (canceled)

24. The method of claim 15, wherein the lymphangiogenic condition comprises corneal lymphangeogenesis and the VAP-1 inhibitor is administered to the subject in an amount sufficient to inhibit corneal lymphangiogenesis.

25. The method of claim 24, wherein inhibition of corneal lymphangiogenesis comprises lymph vessel regression or inhibition of lymph vessel formation.
